(19) United States
(12) Patent Application Publication
(10) Pub. No.: US 2004/0023271 A1
(43) Pub. Date: Feb. 5, 2004

(54) Single primer isothermal nucleic acid amplification-enhanced analyte detection and quantification

(76) Inventors: Nurith Kurn, Palo Alto, CA (US); Geoffrey A. Dafforn, Los Altos, CA (US)

Correspondence Address: MORRISON & FOERSTER LLP, Attn: Catherine M. Polizzi, 755 Page Mill Road, Palo Alto, CA, 94304, US

(21) Appl. No.: 10/404,895

(22) Filed: Mar. 31, 2003

Related U.S. Application Data
(60) Provisional application No. 60/368,628, filed on Mar. 29, 2002.

(51) Int. Cl. C12Q 1/68

(52) U.S. Cl. 435/6

(57) ABSTRACT

The present invention provides novel methods of indirect analyte dectection and quantification through amplification of oligonucleotide template attached to binding partners for analytes by nucleic acid amplification utilizing isothermal, single primer linear nucleic acid amplification methods. Methods for binding of binding partner that is attached to an oligonucleotide template to analyte, then amplifying at least a portion of the oligonucleotide template using a composite primer, primer extension, strand displacement, and optionally a termination sequence, are provided. Methods for amplifying sense RNA using a composite primer, primer extension, strand displacement, optionally template switching, a propromoter oligonucleotide and transcription are also provided. Methods for detecting and quantifying amplification products are also provided. The invention further provides compositions and kits for practicing said methods.

### CROSS-REFERENCE TO RELATED APPLICATION

This application claims priority benefit of the provisional patent application U.S. Serial No. 60/368,628, filed Mar. 29, 2002, which is incorporated by reference in its entirety.

### TECHNICAL FIELD

The invention relates to the field of detection of analytes through amplification of polynucleotide attached to a binding partner. More particularly, the invention provides methods, compositions and kits for binding of analyte binding partners to analyte wherein the binding partner is attached to an oligonucleotide template, and for amplifying (i.e., making multiple copies) the oligonucleotide template, where the methods employ a single RNA/DNA composite primer, with the amplification optionally involving transcription, and detecting amplification products.

### BACKGROUND

Advances in nucleic acid technology have had major effects on various aspects of detection technologies. Automated oligonucleotide synthesis, which incorporates the use of many modified nucleotides, both deoxyribo- and ribo-nucleotides and combinations thereof, has enabled the use of these highly diverse molecules in various applications. Moreover, the large body of knowledge on binding specificity of complementary oligonucleotides, conjugation of oligonucleotides to other molecules and surfaces, has contributed to the wide use of oligonucleotides for many applications, including their use as reporter-groups and capture agents in various analytical procedures for the detection and quantification of single and multiple non-nucleic acid analytes. The ability to amplify oligonucleotide targets in vitro, or to generate multiple copies of reporter-oligonucleotide targets, further enhances their use for enhanced detection.

The enhanced sensitivity of the detection of antibodies bound to antigen by coupling of immune recognition with DNA amplification has been documented previously (Sano, Smith and Cantor, 1992). Immuno-PCR is carried out employing unique DNA sequence tags, which are associated with a specific antigen either covalently or through streptavidin-biotin interactions. Antibody binding to antigens is detected by PCR amplification of the associated DNA tag. The utility of using multiple antibodies and DNA tags was demonstrated by simultaneous analysis of several antigens by immuno-PCR. Although immuno-PCR was shown to be significantly more sensitive than ELISA, the method is limited by drawbacks of PCR such as the requirement for thermal cycling and difficulties in quantification of the amplification products. These limitations have restricted the widespread adoption of immuno-PCR as an alternative to ELISA.

Other methods have been employed for detection technology wherein nucleic acid amplification is used to produce a detectable signal from low levels of analyte. These include the methods described in U.S. Pat. Nos. 6,083,689; 5,985,548; 5,854,033; 5,655,539; and 5,849,478.

### SUMMARY OF THE INVENTION

In one aspect, the invention provides methods for determining presence or absence of an analyte in a sample by a) contacting the sample with a binding partner that is attached to an oligonucleotide template and that is capable of binding, directly or indirectly, to the analyte, if present, under conditions that permit binding, whereby an analyte-binding partner complex is formed if analyte is present; b) separating analyte-binding partner complex from unbound binding partner; c) amplifying a polynucleotide sequence complementary to at least a portion of the oligonucleotide template according to a method comprising: (i) hybridizing a composite primer to the oligonucleotide template, said composite primer comprising an RNA portion and a 3′ DNA portion; (ii) extending the composite primer with DNA polymerase, whereby a primer extension product comprising a detectable identifying characteristic is produced; (iii) cleaving RNA of the hybridized extended composite primer with an enzyme that cleaves RNA from an RNA/DNA hybrid such that another composite primer hybridizes to the oligonucleotide template and repeats primer extension by strand displacement to produce cleaved primer extension product that comprises a detectable identifying characteristic; whereby multiple copies of the polynucleotide sequence complementary to at least a portion of the oligonucleotide template are produced; and whereby detection of the cleaved primer extension product comprising the detectable identifying characteristic indicates presence of the analyte in the sample. In another aspect, the invention provides methods for quantifying an analyte in a sample by performing steps a) to c) above, and also comparing the amount of copies of the polynucleotide sequence complementary to at least a portion of the oligonucleotide template obtained in the sample, if any, to the amount of copies of the polynucleotide sequence complementary to at least a portion of the oligonucleotide template obtained in a reference comprising a known amount of the analyte obtained in a reference comprising a known amount of the analyte that is subjected to steps (a) to (c); whereby the comparison provides quantification of amount of analyte in the sample.

In another aspect, the invention provides methods for determining presence or absence of an analyte in a sample by a) contacting the sample with a binding partner that is attached to an oligonucleotide template and that is capable of binding to the analyte under conditions that permit binding, whereby an analyte-binding partner complex is formed if analyte is present; b) separating analyte-binding partner complex from unbound binding partner; c) amplifying a polynucleotide sequence complementary to at least a portion of the oligonucleotide template in the analyte-binding partner complex by (i) hybridizing a composite primer to the oligonucleotide template attached to the binding partner in the analyte-binding partner complex, said composite primer comprising an RNA portion and a 3′ DNA portion; (ii) extending the composite primer with DNA polymerase, whereby a primer extension product is produced; (iii) cleaving RNA of the hybridized extended composite primer with an enzyme that cleaves RNA from an RNA/DNA hybrid such that another composite primer hybridizes to the oligonucleotide template and repeats primer extension by strand displacement; and (iv) hybridizing a polynucleotide comprising a propromoter and a region which hybridizes to the displaced primer extension product under conditions which allow transcription to occur by RNA polymerase, such that RNA transcripts are produced; whereby multiple copies of the RNA transcripts are produced; and whereby detection a detectable identifying characteristic of the RNA transcripts indicates presence of the analyte in the sample. In another aspect the invention provides methods for quantifying an analyte in a test sample by performing steps (a) to (c) above and also comparing amount of RNA transcripts obtained in the sample to the amount of RNA transcripts obtained in a reference comprising a known amount of the analyte that is subjected to steps (a) to (c); whereby the comparison provides quantification of amount of analyte in the sample.

In another aspect the invention provides methods for determining presence or absence of each of a plurality of analytes in a sample by: a) contacting the sample with a plurality of different binding partners, each of which is attached to an oligonucleotide template and each of which is capable of binding one of the plurality of different analytes under conditions that permit binding, whereby an analyte-binding partner complex is formed for a particular pair of analyte and binding partner if the analyte is present, wherein the oligonucleotide template for each different binding partner comprises a primer-binding region that is common to all of the binding partners and a primer-extension region that is unique for each binding partner; b) separating analyte-binding partner complexes from unbound binding partners; c) amplifying a polynucleotide sequence complementary to at least a portion of each oligonucleotide template present after step b) according to a method comprising: (i) hybridizing a composite primer to the oligonucleotide template, said composite primer comprising an RNA portion and a 3′ DNA portion; (ii) extending the composite primer with DNA polymerase, whereby a unique primer extension product comprising a unique detectable identifying characteristic is produced for each analyte-binding partner complex; (iii) cleaving RNA of the hybridized extended composite primer with an enzyme that cleaves RNA from an RNA/DNA hybrid such that another composite primer hybridizes to the oligonucleotide template and repeats primer extension by strand displacement to produce unique cleaved primer extension product for each different oligonucleotide template that comprises a unique detectable identifying characteristic; whereby multiple copies of the polynucleotide sequence complementary to at least a portion of each oligonucleotide template present after step b) are produced; and whereby detection of the unique detectable identifying characteristic of the polynucleotide sequence complementary to at least a portion of the oligonucleotide template attached to the binding partner for an analyte indicates the presence of the analyte in the sample. In another aspect the invention provides methods for quantifying the relative amounts of each analyte in the sample by comparing the relative amounts of polynucleotide sequence complementary to at least a portion of the oligonucleotide template attached to the binding partner in each analyte-binding partner complex.

In another aspect the invention provides methods for detecting the presence or absence of an analyte in a sample comprising incubating a reaction mixture, said reaction mixture comprising: (a) a sample suspected of containing a complex of the analyte and a binding partner, wherein the binding partner is attached to an oligonucleotide template; (b) a composite primer to the oligonucleotide template, said composite primer comprising an RNA portion and a 3′ DNA portion; (c) a DNA polymerase, dNTPs, and an enzyme that cleaves RNA from an RNA/DNA hybrid; wherein the incubation is under conditions that permit hybridization of the composite primer and the oligonucleotide template, oligonucleotide polymerization, and RNA cleavage, such that multiple copies of the polynucleotide sequence complementary to at least a portion of the oligonucleotide template are produced, and wherein detection of a detectable identifying characteristic of the copies of the polynucleotide sequence complementary to at least a portion of the oligonucleotide template indicates the presence of the analyte.

In another aspect the invention provides methods for detecting the presence or absence of an analyte in a sample comprising incubating a reaction mixture, said reaction mixture comprising: (a) a sample suspected of containing a complex of the analyte and a binding partner, wherein the binding partner is attached to an oligonucleotide template; (b) a composite primer to the oligonucleotide template, said composite primer comprising an RNA portion and a 3′ DNA portion; (c) a DNA polymerase, dNTPs, an enzyme that cleaves RNA from an RNA/DNA hybrid; (d) a polynucleotide comprising a propromoter and a region homologous to a region of the oligonucleotide template; RNA polymerase; and NTP's; wherein the incubation is under conditions that permit hybridization of the composite primer and the oligonucleotide template, oligonucleotide polymerization, and RNA cleavage; and hybridization of the polynucleotide comprising a propromoter and a region homologous to a region of the oligonucleotide template to cleaved primer extension product, to produce a hybridization product comprising a promoter, and transcription of the hybridization product comprising a promoter by the RNA polymerase, whereby multiple RNA transcripts are produced, and wherein detection of the RNA transcripts indicates presence of the analyte.

In another aspect the invention provides methods for generating multiple copies of and/or quantifying a polynucleotide sequence complementary to a polynucleotide sequence attached to a binding partner by: (a) hybridizing a composite primer to the oligonucleotide template attached to the binding partner, said composite primer comprising an RNA portion and a 3′ DNA portion; (b) extending the composite primer with DNA polymerase; (c) cleaving RNA of the hybridized composite primer with an enzyme that cleaves RNA from an RNA/DNA hybrid such that another composite primer hybridizes to the oligonucleotide template and repeats primer extension by strand displacement, whereby multiple copies of the polynucleotide sequence complementary to at least a portion of an oligonucleotide template attached to a binding partner are produced.

In another aspect the invention provides methods for generating multiple copies of RNA transcripts of oligonucleotide template attached to a binding partner by (a) hybridizing a composite primer to the oligonucleotide template, said composite primer comprising an RNA portion and a 3′ DNA portion; (b) extending the composite primer with DNA polymerase; (c) cleaving RNA of the hybridized composite primer with an enzyme that cleaves RNA from an RNA/DNA hybrid such that another composite primer hybridizes to the oligonucleotide template and repeats primer extension by strand displacement to produce a displaced primer extension product; and (d) hybridizing a polynucleotide comprising a propromoter and a region which hybridizes to the displaced primer extension product under conditions that allow transcription to occur by RNA polymerase, such that RNA transcripts are produced comprising sequences complementary to the displaced primer extension products, whereby multiple copies of the polynucleotide sequence complementary to at least a portion of an oligonucleotide template attached to a binding partner are produced.

In one aspect, the invention provides methods for determining presence or absence of an analyte in a sample by: a) contacting the sample with a binding partner capable of binding to the analyte under conditions which permit binding, whereby a complex of analyte and binding partner is formed if analyte is present; wherein the binding partner is attached to an oligonucleotide template; b) optionally, separating the complex, if present, from unbound binding partner; c) amplifying a polynucleotide sequence complementary to a portion of the oligonucleotide template attached to the binding partner in the complex according to a method comprising: (i) hybridizing a composite primer to the oligonucleotide template, said composite primer comprising an RNA portion and a 3′ DNA portion; (ii) optionally, hybridizing a polynucleotide comprising a termination polynucleotide sequence to a region of the oligonucleotide template that is 5′ with respect to hybridization of the composite primer to the oligonucleotide template; (iii) extending the composite primer with DNA polymerase, whereby a primer extension product comprising a detectable identifying characteristic is produced; (iv) cleaving the RNA portion of the hybridized extended composite primer with an enzyme that cleaves RNA from an RNA/DNA hybrid such that another composite primer hybridizes to the oligonucleotide template and repeats primer extension by strand displacement; whereby multiple copies of the polynucleotide sequence complementary to a portion of the oligonucleotide template are produced; and whereby detection of the cleaved primer extension product comprising the detectable identifying characteristic indicates presence of the analyte in the sample.

In another aspect, the invention provides methods for determining presence or absence of an analyte in a sample by: a) contacting the sample with a binding partner capable of binding to the analyte under conditions which permit binding, whereby a complex of analyte and binding partner is formed if analyte is present; wherein the binding partner is attached to an oligonucleotide template; b) optionally, separating the complex, if present, from unbound binding partner; c) amplifying a portion of the oligonucleotide template attached to the binding partner in the complex according to a method comprising: (i) hybridizing a composite primer to the oligonucleotide template, said composite primer comprising an RNA portion and a 3′ DNA portion; (ii) optionally, hybridizing a polynucleotide comprising a termination polynucleotide sequence to a region of the oligonucleotide template that is 5′ with respect to hybridization of the composite primer to the oligonucleotide template; (iii) extending the composite primer with DNA polymerase; (iv) cleaving the RNA portion of the hybridized extended composite primer with an enzyme that cleaves RNA from an RNA/DNA hybrid such that another composite primer hybridizes to the oligonucleotide template and repeats primer extension by strand displacement; (v) hybridizing a polynucleotide comprising a propromoter and a region which hybridizes to the displaced primer extension product under conditions which allow transcription to occur by RNA polymerase, such that RNA transcripts comprising a detectable identifying characteristic are produced; whereby multiple copies of a portion of the oligonucleotide template are produced; and whereby detection of the RNA transcripts comprising the detectable identifying characteristic indicates presence of the analyte in the sample.

In another aspect, the invention provides methods for quantifying an analyte in a test sample by: a) contacting the sample with a binding partner capable of binding to the analyte under conditions which permit binding, whereby a complex of analyte and binding partner is formed if analyte is present; wherein the binding partner is attached to an oligonucleotide template; b) optionally, separating the complex, if present, from unbound binding partner; c) amplifying a polynucleotide sequence complementary to a portion of the oligonucleotide template attached to the binding partner in the complex according to a method comprising: (i) hybridizing a composite primer to the oligonucleotide template, said composite primer comprising an RNA portion and a 3′ DNA portion; (ii) optionally hybridizing a polynucleotide comprising a termination polynucleotide sequence to a region of the oligonucleotide template that is 5′ with respect to hybridization of the composite primer to the oligonucleotide target reporter; (iii) extending the composite primer with DNA polymerase; (iv) cleaving the RNA portion of the hybridized extended composite primer with an enzyme that cleaves RNA from an RNA/DNA hybrid such that another composite primer hybridizes to the oligonucleotide template and repeats primer extension by strand displacement; whereby multiple copies of the polynucleotide sequence complementary to a portion of the oligonucleotide template are produced; and (d) comparing amount of amplified polynucleotide sequence to amount of amplified polynucleotide sequence obtained in a reference sample comprising a known amount of the analyte; whereby the comparison provides quantification of amount of analyte in the test sample.

In another aspect the invention provides methods for quantifying an analyte in a test sample by: a) contacting the sample with a binding partner capable of binding to the analyte under conditions which permit binding, whereby a complex of analyte and binding partner is formed if analyte is present; wherein the binding partner is attached to an oligonucleotide template; b) separating the complex, if present, from unbound binding partner; c) amplifying a polynucleotide sequence complementary to a portion of the oligonucleotide template attached to the binding partner in the complex according to a method comprising: (i) hybridizing a composite primer to the oligonucleotide template, said composite primer comprising an RNA portion and a 3′ DNA portion; (ii) optionally hybridizing a polynucleotide comprising a termination polynucleotide sequence to a region of the oligonucleotide template that is 5′ with respect to hybridization of the composite primer to the oligonucleotide target reporter; (iii) extending the composite primer with DNA polymerase; (iv) cleaving the RNA portion of the hybridized extended composite primer with an enzyme that cleaves RNA from an RNA/DNA hybrid such that another composite primer hybridizes to the oligonucleotide template and repeats primer extension by strand displacement; (v) hybridizing a polynucleotide comprising a propromoter and a region which hybridizes to the displaced primer extension product under conditions which allow transcription to occur by RNA polymerase, such that RNA transcripts comprising a detectable identifying characteristic are produced; whereby multiple copies of a portion of the oligonucleotide template are produced; and (d) comparing amount of amplified portion of the oligonucleotide template to amount of amplified portion of the oligonucleotide template obtained in a reference sample comprising a known amount of the analyte; whereby the comparison provides quantification of amount of analyte in the test sample.

In another aspect the invention provides methods for detecting the presence of an analyte in a sample comprising incubating a reaction mixture, said reaction mixture comprising: (a) a complex of the analyte and a binding partner, wherein the binding partner is attached to an oligonucleotide template; (b) a composite primer to the oligonucleotide template, said composite primer comprising an RNA portion and a 3′ DNA portion; (c) optionally, a polynucleotide comprising a termination polynucleotide sequence to a region of the oligonucleotide template that is 5′ with respect to hybridization of the composite primer to the oligonucleotide template; (d) a DNA polymerase, dNTPs, and an enzyme that cleaves RNA from an RNA/DNA hybrid; wherein the incubation is under conditions that permit hybridization of the composite primer and the oligonucleotide template, oligonucleotide polymerization, and RNA cleavage, such that a cleaved primer extension product comprising a detectable identifying characteristic is produced, and wherein the cleaved primer extension product is detected, whereby the presence of the analyte is detected.

In another aspect the invention provides methods for detecting the presence of an analyte in a sample comprising incubating a reaction mixture, said reaction mixture comprising: (a) a complex of the analyte and a binding partner, wherein the binding partner is attached to an oligonucleotide template; (b) a composite primer to the oligonucleotide template, said composite primer comprising an RNA portion and a 3′ DNA portion; (c) optionally, a polynucleotide comprising a termination polynucleotide sequence to a region of the oligonucleotide template that is 5′ with respect to hybridization of the composite primer to the oligonucleotide template; (d) a DNA polymerase, dNTPs, an enzyme that cleaves RNA from an RNA/DNA hybrid; (e) a polynucleotide comprising a propromoter and a region homologous to a region of the oligonucleotide template; RNA polymerase; and NTP's; wherein the incubation is under conditions that permit hybridization of the composite primer and the oligonucleotide template, oligonucleotide polymerization, and RNA cleavage; and hybridization of the polynucleotide comprising a propromoter and a region homologous to a region of the oligonucleotide template to cleaved primer extension product, to produce a hybridization product comprising a promoter, and transcription of the hybridization product comprising a promoter by the RNA polymerase, whereby multiple copies of a portion of the olignonucleotide template comprising a detectable identifying characteristic are produced, and wherein the olignonucleotide template is detected, whereby the presence of the analyte is detected.

In another aspect the invention provides methods for generating multiple copies of and/or quantifying a polynucleotide sequence complementary to a polynucleotide sequence attached to a binding partner by: (a) hybridizing a single stranded DNA template with a composite primer, said composite primer comprising an RNA portion and a 3′ DNA portion, wherein the single stranded DNA template is attached to a binding partner; (b) extending the composite primer with DNA polymerase; (c) cleaving the RNA portion of the annealed composite primer with an enzyme that cleaves RNA from an RNA/DNA hybrid such that another composite primer hybridizes to the template and repeats primer extension by strand displacement, whereby multiple copies of the complementary sequence of the polynucleotide sequence attached to the binding partner are produced.

In another aspect the invention provides methods for generating multiple copies of a polynucleotide sequence attached to a binding partner by:(a) hybridizing a single stranded DNA template with a composite primer, said composite primer comprising an RNA portion and a 3′ DNA portion, wherein the single stranded DNA template is attached to a binding partner; (b) extending the composite primer with DNA polymerase; (c) cleaving the RNA portion of the annealed composite primer with an enzyme that cleaves RNA from an RNA/DNA hybrid such that another composite primer hybridizes to the template and repeats primer extension by strand displacement to produce displaced primer extension product; (d) hybridizing a polynucleotide comprising a propromoter and a region which hybridizes to the displaced primer extension product under conditions which allow transcription to occur by RNA polymerase, such that RNA transcripts are produced comprising sequences complementary to the displaced primer extension products, whereby multiple copies of the polynucleotide sequence attached to the binding partner are produced.

In another aspect, the invention provides methods of determining presence or absence of an analyte in a sample by extending a composite primer comprising an RNA portion and a 3′ DNA portion in a complex comprising: (a) an oligonucleotide template attached to a binding partner bound to an analyte; and (b) a primer extension product produced by extension of a composite primer comrpsing an RNA portion and a 3′ DNA portion, wherein RNA from the extension product has been cleaved by an enzyme that cleaves RNA from an RNA/DNA hybrid such that the composite primer can hybridze to the oliognucleotide template and be extended by DNA polymerase, whereby the cleaved primer extension product is displaced, and whereby detection of the cleaved primer extension product (which comprises a detectable identifying characteristic) indicates presence of the analyte in the sample.

In some embodiments of the foregoing methods, the analyte may be bound to an intermediate binding partner, binding of the analyte to an intermediate binding partner, wherein the intermediate binding partner binds to the binding partner that is attached to the oligonucleotide template, whereby the binding partner that is attached to an oligonucleotide template indirectly binds to analyte via the intermediate binding partner instead of by binding directly to the analyte, and whereby an analyte-binding partner complex is formed. For example, the intermediate binding partner may comprise an antibody specific to the analyte, for example, the analyte may, in some embodiments, comprise a member of the Botulinum toxin (BoNT) family. In some embodiments of the foregoing methods, the binding partner that has an attached oligonucleotide template may comprise secondary antibody specific to the intermediate binding partner antibody, for example, a secondary antibody to the antibody specific for a member of the BoNT family.

In some embodiments of the foregoing methods, the analyte may comprise a structure chosen from the group consisting of proteins, polypeptides, peptides, nucleic acid segments, carbohydrates, cells, microorganisms and fragments and products thereof, an organic molecule, and an inorganic molecule. The analyte may, for example, comprise a peptide, for example a member of the Botulinum toxin (BoNT) family.

In some embodiments of the foregoing methods, the RNA portion of the composite primer is 5′ with respect to the 3′ DNA portion, and in some of these embodiments, the 5′ RNA portion is adjacent to the 3′ DNA portion. In some embodiments of the foregoing mehods, the oligonucleotide template comprises a ssDNA portion, wherein the ssDNA portion has a length of about 25 to about 100 nucleotides, and in some embodiments the ssDNA portion has a length of about 25 to about 200 nucleotides.

In some embodiments of the foregoing methods, the enzyme that cleaves RNA is RNase H.

In some embodiments of the foregoing methods, the oligonucleotide template is covalently attached to the binding partner. In other embodiments of the foregoing methods, the oligonucleotide template is non-covalently attached to the binding partner.

In some embodiments of the foregoing methods, the detectable identifying characteristic is selected from the group consisting of size of the cleaved primer extension product or RNA transcript, sequence of the cleaved primer extension product or RNA transcript, and detectable signal associated with the cleaved primer extension product or RNA transcript. In some of these embodiments, the detectable identifying characteristic may comprise the sequence of the cleaved primer extension product or RNA transcript, wherein the sequence is detected by hybridizing the cleaved primer extension product or RNA transcript with a nucleic acid probe that is hybridizable to the cleaved primer extension product or RNA transcript, for example, the nucleic acid probe may comprise DNA. In some embodiments, the nucleic acid probe is provided as an array, for example wherein the array comprises the probe immobilized on a substrate fabricated from a material selected from the group consisting of paper, glass, plastic, polypropylene, nylon, polyacrylamide, nitrocellulose, silicon, metal, polystyrene, and optical fiber.

In some embodiments of the foregoing methods, the detectable signal is associated with a label on a deoxyribonucleoside triphosphate or ribonucleoside triphosphate or analog thereof that is incorporated during primer extension or during transcription.

In some embodiments of the foregoing methods, the detectable signal is associated with interaction of two labels, wherein the labels are on deoxynucleoside triphosphates or ribonucleoside triphosphates or analogs thereof, and wherein one or both of the labels is incorporated during primer extension or during RNA transcription. In some of these embodiments, one label is on a deoxyribonucleoside triphosphate or analog thereof that is incorporated during primer extension and another label is on a deoxyribonucleoside triphosphate or analog thereof located in the primer portion of the primer extension product.

In some embodiments of the foregoing methods, the analyte-binding partner complex, if present, is separated from unbound binding partner by capture of the analyte on a solid surface that comprises a capture partner specific for said analyte.

In some embodiments of the foregoing methods, the analyte(s) or analyte-binding partner complex(s) is attached to a solid surface. In other embodiments of the foregoing methods, the analyte(s) analyte-binding partner complex(s) is in solution.

As this disclosure makes clear, contacting a sample includes contacting an analyte in the sample, if present.

The invention also provides compositions (including complexes) as well as kits.

### BRIEF DESCRIPTION OF THE DRAWINGS

FIG. 1 shows a detectable complex of an analyte and a binding partner, where the binding partner is attached to an oligonucleotide template.

FIG. 2 shows capture of an analyte-binding partner complex on a solid surface.

FIG. 3 shows a complex formed by an analyte that is bound to an intermediate binding partner, which is bound to a binding partner attached to an oligonucleotide template.

FIG. 4 shows capture of the complex of FIG. 3 on a solid support.

FIG. 5 shows a “multiplex” analyte (termed herein as a “multisubunit analyte” or “multiple-subunit analyte”), composed of two different subunits, bound to two different binding partners, each of which is attached to an oligonucleotide template.

FIG. 6 shows a single analyte bound to two binding partners, each of which is attached to an oligonucleotide template.

FIG. 7 shows an analyte-binding partner complex of analyte and three binding partners, two of which are attached to oligonucleotide templates and the third of which is attached to a capture moiety (which can be, for example, a capture oligonucleotide).

FIG. 8 shows capture of the complex of FIG. 7 on a solid support by binding of the capture moiety of the analyte-binding partner complex to its corresponding capture moiety on the support.

FIGS. 9A-C shows enhanced amplification by RNA transcription to produce sense RNA, utilizing single primer isothermal amplification (SPIA™) and a template switch oligonucleotide containing a propromoter to produce dsDNA for transcription.

FIGS. 10A-C shows single primer isothermal linear amplification (SPIA™) of nucleic acid to produce ssDNA that is complementary to the amplified strand, using a blocker sequence.

FIGS. 11A-D shows enhanced single primer isothermal linear amplification of nucleic acid using a propromoter template oligonucleotide and RNA polymerase to produce sense RNA.

FIGS. 12A and 12B show single primer isothermal amplification (SPIA™) of an oligonucleotide template.

### MODES FOR CARRYING OUT THE INVENTION

Overview of the Invention and its Advantages

The invention provides methods, compositions and kits for ultra sensitive detection and quantification of the interaction of two or more molecules to form a specific complex. In particular, the invention discloses novel methods, compositions and kits for ultrasensitive detection and/or quantification of analyte(s). The invention further provides methods, compositions and kits for detection and quantification of multiple-subunit analytes and interaction of multiple binding partners with single or multiple analytes.

The methods generally comprise using a binding partner to the analyte, where the binding partner is attached to an oligonucleotide template, and further generally comprise amplifying a portion of the oligonucleotide template using an RNA/DNA composite primer, optionally a termination sequence, and, in embodiments in which transcription is used, a propromoter oligonucleotide sequence, to produce amplification product that comprises a detectable identifying characteristic. As this disclosure makes clear, reference to amplifying “a portion” of the template refers to amplifying at least a portion of the template, and that at least a portion (but not necessarily only a portion) of the template is amplified. Reference to amplifying a sequence complementary to “a portion” of the oligonucleotide template generally refers to amplifying a sequence complementary to the oliognucleotide template, without implying that the complementary sequence corresponds to the entire oligonucleotide template.

Amplification products are characterized to determine the presence and/or quantity of the analyte in a sample. Conversely, lack of, or insignificant amounts of, amplification products indicates absence of the analyte in a sample. “Absent” or “absence” of product, and “lack of detection of product” as used herein includes insignificant, or de minimus levels, generally due to lack of significant accumulation of product.

As a general summary, the methods work as follows: a binding partner is provided that binds to analyte, if present, to form an analyte-binding partner complex. In some embodiments, a single analyte is detected using a single binding partner. In other embodiments, multiple sites on a single analyte are detected using multiple binding partners. In yet other embodiments, multiple subunit analyte is detected using a single or multiple binding partners. In still other embodiments, multiple analytes are detected using a single binding partner. In yet still other embodiments, multiple analytes are detected using multiple (different) binding partners. In other embodiments, analyte binds to one or more intermediate binding partners, one or more of which bind to a binding partner attached to an oligonucleotide template.

In some embodiments, after binding partner binds to analyte, unbound binding partner is removed or separated from bound binding partner. It is understood that, for purposes of this invention, removal need not be complete and absolute removal, but removal or separation sufficient to permit assessment of presence or absence of analyte (i.e., without significant interference or “noise” arising from amplification of oligonucleotide template attached to unbound binding partner). Alternatively, in other embodiments the two are not separated but only bound binding partner is available for amplification of its oligonucleotide template. A portion of the oligonucleotide template attached to the binding partner is then amplified.

As a general summary, the amplification methods work as follows: a composite RNA/DNA primer forms the basis for replication of at least a portion of the oligonucleotide template. In some embodiments, a termination sequence provides the basis for an endpoint for the replication by either diverting or blocking further replication along the oligonucleotide template, but this is optional. As described below, in some embodiments, the polynucleotide comprising a termination sequence is a template switch oligonucleotide (TSO), which contains sequences that are not of sufficient complementarity to hybridize to the oligonucleotide template (in addition to sequences which are of sufficient complementary to hybridize); in other embodiments, the termination sequence comprises primarily sequences that are of sufficient complementarity to hybridize to the oligonucleotide template. DNA polymerase effects copying of the portion of the oligonucleotide template from the primer. An enzyme which cleaves RNA from an RNA/DNA hybrid (such as RNase H) cleaves (removes) RNA sequence from the hybrid, leaving sequence on the oligonucleotide template available for binding by another composite primer. Another strand is produced by DNA polymerase, which displaces the previously replicated strand, resulting in displaced extension product that is ssDNA complementary to at least a portion of the oligonucleotide template. In some embodiments, a second composite primer may bind to the displaced extension product and replicate it, resulting in a ssDNA that is identical to the original portion of the oligonucleotide template. It will be understood by those of skill in the art that “identical,” in this context, includes ssDNA that contains non-identical bases introduced through normal errors in the polymerization process. Optionally, a polynucleotide comprising a propromoter and a region which hybridizes to the displaced primer extension product (which can be, for example, a template switch oligonucleotide or propromoter template oligonucleotide) that contains sequences of sufficient complementarity to hybridize to the 3′ end of the displaced extension product, binds to the displaced primer extension product. The promoter drives transcription (via DNA-dependent RNA polymerase) to produce sense RNA products.

Accordingly, the amplification methods of the invention provide methods of producing at least one copy of a portion of an oligonucleotide template (generally, methods of amplifying a portion of an oligonucleotide template) comprising combining and reacting the following: (a) a single-stranded oligonucleotide template (attached to a binding partner) comprising a portion to be amplified; (b) a composite primer comprising an RNA portion and a 3′ DNA portion; (c) a DNA polymerase; (d) deoxyribonucleoside triphosphates or suitable analogs; (e) an enzyme, such as RNaseH, which cleaves RNA from an RNA/DNA duplex; (f) optionally, a second primer comprising an RNA portion and a 3′ DNA portion; and (g) optionally, a polynucleotide comprising a termination sequence, such as any of those described herein, which comprises a portion (or region) which hybridizes to the oligonucleotide template. The termination sequence is optional, however, as the oligonucleotide template is synthetic and thus may contain, as part of the ssDNA of the template, termination sites or other sites that cause polymerization to cease. The combination is subjected to suitable conditions such that (a) the composite primer (and, optionally, a polynucleotide comprising a termination sequence) hybridizes to the oligonucleotide template; (b) primer extension occurs from the composite primer, to form a duplex; (c) RNaseH cleaves RNA of the composite primer from the RNA/DNA duplex; (d) another composite primer hybridizes to the oligonucleotide template, and another round of primer extension (mediated by DNA polymerase) occurs, displacing the strand already copied from the template; (e) optionally, the second composite primer hybridizes to the displaced primer extension product and steps (a) through (d) are repeated on the displaced primer extension product.

Optionally, the following is also included in the amplification reaction (either at the same time as those components listed above or added separately): (f) a polynucleotide comprising a propromoter sequence (which can be in any of a number of forms, as described herein) and a region which hybridizes to the displaced primer extension product; (g) ribonucleoside triphosphates or suitable analogs; and (h) RNA polymerase, under conditions such that transcription of the displaced strand can occur. Details regarding the various components of the methods of the present invention are provided below.

The amplification step of the invention provides amplification product corresponding to a portion of the oligonucleotide template that has been multiplied manyfold (e.g., 10¹²-fold).

Detection of analyte (if present) is through detection of product formation, which may be indicated and detected in a number of ways that arise from the process of primer extension. In some embodiments, detection of analyte is through detection of a detectable identifying characteristic of the amplification product. In these embodiments, the amplification product comprises a detectable identifying characteristic. Examples of such detectable identifying characteristics are size, sequence, and label. These may be used singly or in combination in the detection and/or quantification of the amplification product. When size is used, various methods known in the art may be used to characterize the size of the amplification product, such as electrophoresis and other techniques described herein. When sequence is used, the amplification products may be detected by any means known in the art of detecting an oligonucleotide sequence. For example, in some embodiments the method of detection and/or quantification is hybridization to complementary oligonucleotide(s) immobilized on a solid support. Alternatively, in some embodiments, detection is accomplished by detecting pyrophosphate released during amplification of the oligonucleotide template. Thus, detection of product can include indirect detection such as detection of pyrophosphate, or other indicia of extension.

When a label is used as a detectable identifying characteristic, the optical properties of a label may be altered subsequent to attachment to the primer. For example, fluorescence polarization of fluorescent dyes attached to free nucleotide triphosphates has been shown to change upon attachment to a primer by a polymerase. When the amplification product is labeled and the method of detection is immobilization on a solid support, the immobilization of the amplification product on the solid support and detection of the label indicates the presence of the product in the reaction mixture. It is also possible to detect altered spectral properties of a label by means of energy transfer. When the primer is labeled by a donor or acceptor dye, and/or the nucleotide triphosphates, or their analogs, are labeled with acceptor or donor dyes, respectively, the incorporation of the dyes into an amplification product enables energy transfer between the donor and acceptor dyes, thus resulting in specific spectral properties of the attached dyes. Fluorescence dyes useful for this detection mode are known in the art and described herein.

The methods of the invention are further useful for multiple analysis of analyte-binding partner complexes. That is to say, various oligonucleotide templates that are attached to different binding partners that bind to different analytes, may be amplified simultaneously in a single reaction mixture. In such embodiments, the composite primer binding regions of the template oligonucleotides may be the same or different. If the same composite binding region is used, only a single composite primer need be provided, and the relative quantities of various analytes in the analyte-binding partner complexes is more accurately determined.

The methods of the invention further provide for halting the reactions at various timepoints, and the further reaction of the complexes and mixtures that are produced at each timepoint.

The invention also provides compositions, kits, and systems useful in the methods of the invention. As described herein, the invention further provides complexes that comprise, for example, an analyte and a binding partner that is attached to an oligonucleotide template.

Other methods which use the methods and the amplified products described herein are provided below.

Advantages of the Invention

The methods of the invention provide several significant advantages over other methods of analyte detection and/or quantification using nucleic acid amplification. The formation of primed oligonucleotide template, primer extension and displacement of the previously generated extension product is dependent on the cleavage of RNA of the hybridized primer by a ribonuclease activity. Thus, the primer extension product is lacking the 5′-most portion of the primer. Consequently, if a second round of replication is used, or if transcription-based methods are used, the second DNA replication product or the RNA transcription product does not contain at its 3′ end the sequence complementary to this portion of the primer. Thus, the amplification products are not capable of hybridizing to the (first) primer for productive amplification, making the amplification methods of the invention resistant to non-specific amplification due to contamination with products generated by prior amplifications reactions. This feature clearly distinguishes it from other known methods of analyte detection and/or quantification that rely on non-linear nucleic acid amplification, such as PCR, NASBA and the like, and renders the methods of the invention suitable for open tube platforms commonly used in clinical laboratories, high throughput testing sites, and the like.

The methods of the invention do not require thermocycling in order to detect and/or quantify analyte, in that amplification can be performed isothermally. This feature provides numerous advantages, including facilitating automation and adaptation for high throughput analyte detection and/or quantification. Other methods that have been reported require thermal cycling for the separation of amplification products from the original sequence. The isothermal reaction is faster than that afforded by thermal cycling and is suitable for miniaturized devices.

Another advantage of the analyte detection/quantification methods of the invention is that only a single primer is required. A single primer is utilized to provide unidirectional primer extension that results in amplification of a portion of the oligonucleotide template. This obviates the numerous drawbacks associated with having to use primer pairs, for example cost of designing and making two sets of primers and the increased probability that amplified products are the result of non-specific priming.

The products of the amplification according to the methods of the invention are single stranded and are readily detectable by various known nucleic acid detection methods. It is understood that, generally, “detection” of a product (such as a cleaved amplification product comprising a detectable identifying characteristic) means detection of significant amounts of product arising from cycling (i.e., repeated cycles of a reaction). The cycling results in accumulated amplification product. Lack of cycling (due to, or example, absence of an analyte, and thus absence of an analyte-binding partner complex) results in de minimus, or insignificant amount of product which, for purposes of the methods of the invention, is not “detected”.

The methods are also suitable for quantitative determinations. Quantification of the accumulated amplification product permits quantification of the analyte or analytes in the sample.

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984), “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Primers, oligonucleotides and polynucleotides employed in the present invention can be generated using standard techniques known in the art.

Definitions

“Amplification,” as used herein, generally refers to the process of producing multiple copies of a desired sequence. “Multiple copies,” as used herein, means at least 2 copies. A “copy” does not necessarily mean perfect sequence complementarity or identity to the template sequence. A “copy” includes a nucleic acid sequence that is hybridizable (preferably complementary) to the sequence of interest; e.g., to the portion of the oligonucleotide template to be amplified. Copies can include nucleotide analogs such as deoxyinosine, intentional sequence alterations (such as sequence alterations introduced through a primer comprising a sequence that is hybridizable, but not complementary, to the template), and/or sequence errors that occur during DNA polymerization.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, intemucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.oligonucleotide

“Oligonucleotide,” as used herein, generally refers to short, generally single stranded, generally synthetic polynucleotides that are generally, but not necessarily, less than about 200 nucleotides in length. Oligonucleotides in the present invention include the composite primer(s), TSO, PTO and blocker sequence. The terms “oligonucleotide” and “polynucleotide” are not mutually exclusive. The description above for polynucleotides is equally and fully applicable to oligonucleotides.

“Oligonucleotide template,” as used herein, refers to an oligonucleotide, a portion of which serves as a template for a DNA polymerase to produce a strand complementary to the portion of the template strand. Generally an oligonucleotide template is attached to a binding partner.

A “primer” is generally a short single-stranded polynucleotide (often referred to as an oligonucleotide), generally with a free 3′-OH group, that binds to a particular polynucleotide. It can be used to promote polymerization of a polynucleotide complementary to the original polynucleotide.

A “termination polynucleotide sequence” or “termination sequence,” as used interchangeably herein, is a polynucleotide sequence which effects cessation of DNA replication by DNA polymerase with respect to the oligonucleotide template. A termination sequence comprises a portion (or region) that generally hybridizes to the template at a location 5′ to the termination point (site). The hybridizable portion (e.g., the portion that hybridizes) may or may not encompass the entire termination sequence. Examples of suitable termination polynucleotide sequences (such as blocker sequences and TSOs) are provided herein. Termination sequences are optional in the methods of the present invention.

“Blocker sequence,” or “blocking sequence” as used interchangeably herein, is an example of a termination sequence, and refers to an oligonucleotide that binds, generally with high affinity, to the oligonucleotide template nucleic acid at a location 5′ to the termination site and effects cessation of DNA replication by DNA polymerase with respect to the template comprising the target sequence. Its 3′ end may or may not be blocked for extension by DNA polymerase. Blocker sequences are optional in the methods of the present invention.

“Termination site,” or “termination point,” as used interchangeably herein, refers to the site, point or region of the oligonucleotide template that is last replicated by the DNA polymerase before termination of polymerization (generally, primer extension) or template switch. Optionally, for example, with respect to a TSO, it is the position or region in the oligonucleotide template that is complementary to the 3′ end of the primer extension product prior to switching template from the template polynucleotide to the unhybridized portion of the TSO.

“Protopromoter sequence,” and “propromoter sequence,” as used herein, refer to a single-stranded DNA sequence region which, in double-stranded form is capable of mediating RNA transcription. In some contexts, “protopromoter sequence,” “protopromoter,” “propromoter sequence,” “propromoter,” “promoter sequence,” and “promoter” are used interchangeably.

“Template switch oligonucleotide (TSO),” as used herein, refers to an oligonucleotide that comprises a portion (or region) that is hybridizable (e.g., that hybridizes) to a template at a location 5′ to the termination site of primer extension and that is capable of effecting a template switch in the process of primer extension by a DNA polymerase. TSOs are generally known in the art. “Template switch” refers to a change in template nucleic acid, generally from the target nucleic acid to the unhybridized portion of a TSO, during the course of a single round of primer extension. The use of a TSO in the methods of the invention is optional.

“Propromoter template oligonucleotide (PTO),” as used herein, refers to an oligonucleotide that comprises a propromoter sequence and a portion, generally a 3′ portion, that is hybridizable (e.g., that hybridizes) to the 3′ region of a primer extension product. The propromoter sequence and the hybridizable portion may be the same, distinct or overlapping nucleotides of an oligonucleotide.

A “complex” is an assembly of components. A complex may or may not be stable and may be directly or indirectly detected. For example, as is described herein, given certain components of a reaction, and the type of product(s) of the reaction, existence of a complex can be inferred. For purposes of this invention, a complex is generally an intermediate with respect to the final reaction product(s).

A “system,” as used herein, includes a device, apparatus or machinery (e.g., automated) for carrying out the methods of the invention.

A “portion” or “region,” used interchangeably herein, of a polynucleotide or oligonucleotide is a contiguous sequence of 2 or more bases. In other embodiments, a region or portion is at least about any of 3, 5, 10, 15, 20, 25 contiguous nucleotides.

A “reaction mixture” is an assemblage of components, which, under suitable conditions, react to form a complex (which may be an intermediate) and/or a product(s).

“A”, “an” and “the”, and the like, unless otherwise indicated include plural forms.

In accordance with a well-established principle of patent law, “comprising” means including.

Conditions that “allow” or “permit” an event to occur or conditions that are “suitable” for an event to occur, such as hybridization, primer extension, oligonucleotide ligation and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, primer extension.

“Microarray” and “array,” as used interchangeably herein, refer to an arrangement of a collection of nucleotide sequences in a centralized location. Arrays can be on a solid substrate, such as a glass slide, or on a semi-solid substrate, such as nitrocellulose membrane. The nucleotide sequences can be DNA, RNA, or any permutations thereof.

The term “3′” generally refers to a region or position in a polynucleotide or oligonucleotide 3′ (downstream) from another region or position in the same polynucleotide or oligonucleotide.

The term “5′” generally refers to a region or position in a polynucleotide or oligonucleotide 5′ (upstream) from another region or position in the same polynucleotide or oligonucleotide.

The term “3′-DNA portion,” “3′-DNA region,” “3′-RNA portion,” and “3′-RNA region,” refer to the portion or region of a polynucleotide or oligonucleotide located towards the 3′ end of the polynucleotide or oligonucleotide, and may or may not include the 3′ most nucleotide(s) or moieties attached to the 3′ most nucleotide of the same polynucleotide or oligonucleotide. The “3′ most nucleotide” (singular form) refers to the 3′ last nucleotide of a polynucleotide or oligonucleotide. The 3′ most nucleotides (plural form) includes the 3′ most nucleotide and can be preferably from about 1 to about 20, more preferably from about 3 to about 18, even more preferably from about 5 to about 15 nucleotides.

The term “5′-DNA portion,” “5′-DNA region,” “5′-RNA portion,” and “5′-RNA region,” refer to the portion or region of a polynucleotide or oligonucleotide located towards the 5′ end of the polynucleotide or oligonucleotide, and may or may not include the 5′ most nucleotide(s) or moieties attached to the 5′ most nucleotide of the same polynucleotide or oligonucleotide. The “5′ most nucleotide” (singular form) refers to the 5′ first nucleotide of a polynucleotide or oligonucleotide. The 5′ most nucleotides (plural form) includes the 5′ most nucleotide and can be preferably from about 1 to about 20, more preferably from about 3 to about 18, even more preferably from about 5 to about 15 nucleotides.

“Hybridizable,” or “capable of hybridizing” as used herein, refers to the capability and/or ability of two polynucleotide sequences to hybridize through at least some degree of complementary base pairing, under conditions used in any of the methods described herein; i.e., at the temperature, pH, ionic concentrations, and the like, used in carrying out the methods of the invention. As such, a sequence (such as a primer) which is hybridizable to another sequence (such as an oligonucleotide template) hybridizes to that sequence under suitable conditions.

“Detectable identifying characteristic,” as used herein, refers to characteristics of a reaction product that indicates its presence, wherein the characteristic is detectable by methods known in the art.

“Detection” includes any means of detecting, including direct and indirect detection. For example, “detectably fewer” products may be observed directly or indirectly, and the term indicates any reduction (including no products). Similarly, “detectably more” product means any increase, whether observed directly or indirectly.

The term “analyte” as used herein refers to a substance to be detected or assayed by the methods of the present invention, for example, a compound whose properties, location, quantity, presence, absence, and/or identity is desired to be characterized. Typical analytes may include, but are not limited to proteins, polypeptides, peptides, nucleic acid segments, carbohydrates, cells, microorganisms and fragments and products thereof, organic molecules, inorganic molecules, or any substance for which attachment sites for binding partner(s) can be developed. In a related application, “analyte” also referred to a moiety that serves to indirectly detect a structure of interest, by binding (either directly or indirectly) to a structure of interest (thus, more properly, “analyte” in this context refers to an “intermediate binding partner”; for clarity, and in accordance with what is conveyed herein, former reference to “structure of interest” has been replaced by “analyte”; and, where intermediate binding partners are used, former reference to “analyte” has been replaced by reference to “intermediate binding partner”).

It is understood that, with respect to all embodiments described herein, as generally “comprising” components or aspects, the invention also includes embodiments which “consist essentially of” these components or aspects. The invention also includes embodiments which “consist of” these components or aspects. This applies to all embodiments described herein.

Methods of the Invention

I. Binding of Analyte and Binding Partner

A. Components

1. Analyte

Generally, the first reaction in the methods of the invention is the binding of an analyte or analytes to a binding partner or binding partners to form an analyte-binding partner complex. One or more of the binding partners in the analyte-binding partner complex is attached to oligonucleotide template(s), the amplification of a portion of which provides the signal indicating the presence of the analyte.

The analyte that forms a complex with a binding partner or binding partners may be any substance or combination of substances whose characteristics are sought to be determined. Such characteristics include, but are not limited to presence, absence, quantity, state of assembly, conformational state, or binding state.

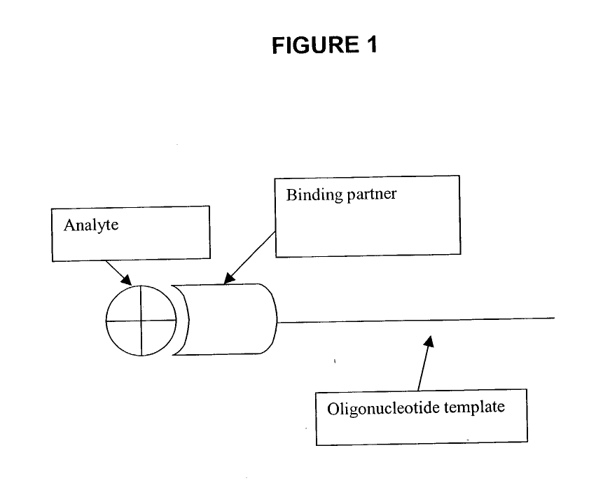

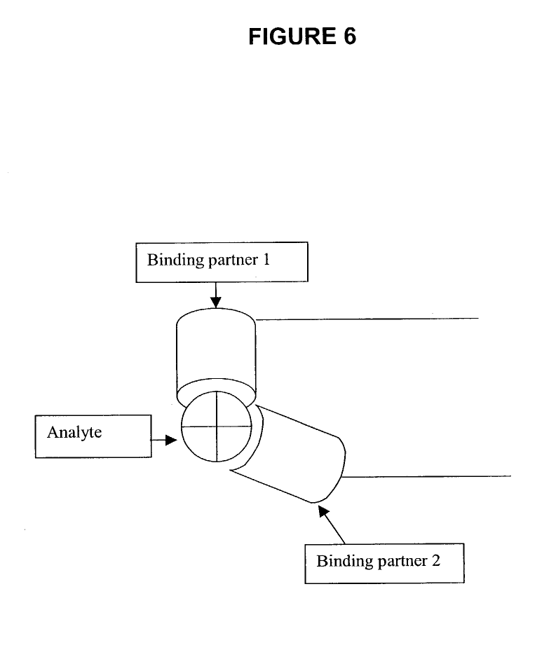

In some embodiments of the methods of the invention, the analyte includes one binding site for a binding partner (see FIG. 1). In some embodiments the analyte includes multiple binding sites for multiple binding partners; in these embodiments the binding sites may be for the same or different binding partners (see FIG. 6). It will be appreciated that if an analyte possesses multiple binding sites, the accessibility of the binding sites for their respective binding partners will determine the nature of the complex formed upon binding of analyte to binding partner(s), and the composition of the complex may be detected and provide information as to the state of the analyte. For example, an analyte that is a ligand may possess two or more binding sites, one or more of which is blocked upon binding of the ligand to its receptor; the presence of complexes of ligand with one or more binding partners may be detected, allowing one to determine presence or absence of ligand, presence or absence of binding of ligand to receptor, and relative and/or absolute quantities of bound versus unbound ligand.

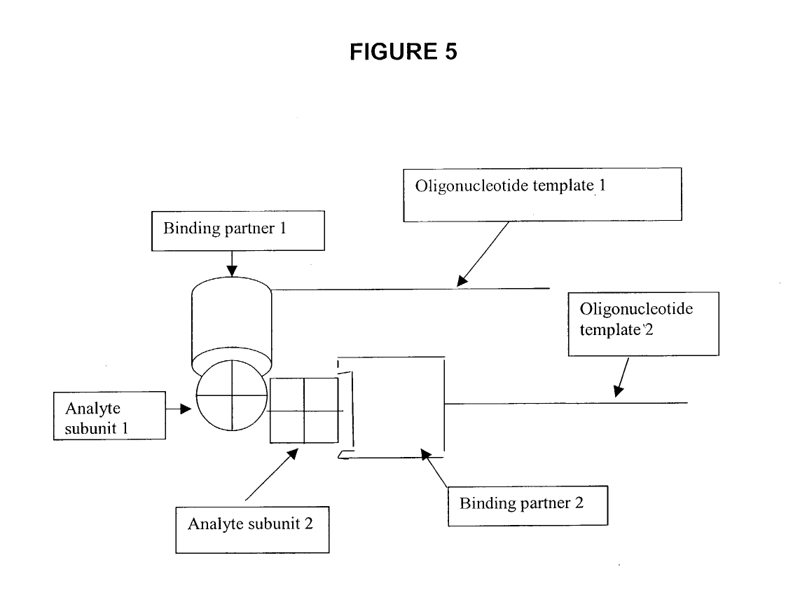

In some embodiments the analyte is a single entity, and in other embodiments multiple units may be in association to form a multisubunit analyte or multiple-subunit analyte (see FIG. 5). Examples of multisubunit analytes include, but are not limited to, multisubunit proteins (including multisubunit enzymes), replication and repair complexes where multiple proteins interact with a polymerase or a repair enzyme, assemblies of large and small ribosomal subunits, and other multisubunit assemblies or molecular or supramolecular compositions which may vary in structure and size. The composition and/or organization of a multisubunit analyte may change with changing conditions in the environment in which it is located. The environment may be in a non-living or living system; examples of environments in living systems include supramolecular assemblies, organelles, cytoplasm, nucleoplasm, membranes, cells, tissues, organs, and/or organisms. The methods of the invention allow one to determine the state of organization of the multisubunit analyte, e.g., the stage of assembly of a supramolecular complex. In some embodiments, the state of organization may be detected by providing multiple binding partners, each of which is specific for a different unit of the multi-subunit analyte. In some embodiments, the state of organization may be detected by providing binding partners that bind to conformations that correspond to different states of organization of the multisubunit analyte. An example of the latter is where the multi-subunit analyte is a multisubunit protein, where the subunits must be associated in order to form the site that the binding partner binds to. Thus, binding of binding partner indicates the association of the subunits into the multisubunit protein.

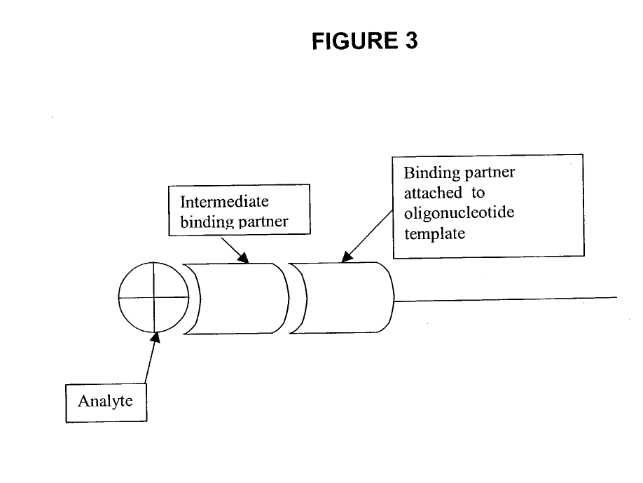

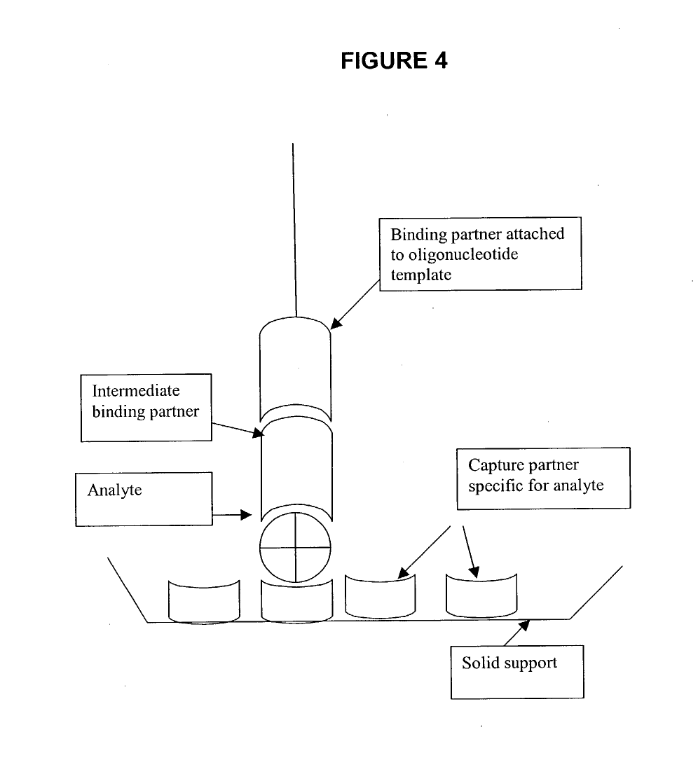

In some embodiments an intermediate binding partner binds to the analyte, and a second binding partner, to which oligonucleotide template is attached, binds to the intermediate binding partner (see FIGS. 3 and 4). It will be appreciated that there may also be any number of intermediate binding partners between analyte and the binding partner that is attached to an oligonucleotide template. It will also be appreciated that the binding partner that is attached to an oligonucleotide template in these “sandwich” embodiments may be specific for a class of intermediate binding partners rather than for a single intermediate binding partner. Thus, for example, the intermediate binding partner in a sandwich assay may be a monoclonal antibody specific for a particular hapten, while the binding partner that is attached to an oligonucleotide template may be a secondary antibody that is specific for a class of antibodies of which the intermediate binding partner antibody is a member, e.g., an anti-IgG antibody. Other intermediate binding partners for which the binding partner attached to an oligonucleotide template is specific may be specific for other haptens. Such sandwich methods allow the detection of any of a number of analytes using a class of intermediate binding partners, each of which is specific for each individual analyte, and a single binding partner attached to an oligonucleotide template, specific for the entire class of intermediate binding partners, obviating the necessity for attaching oligonucleotide templates to multiple binding partners specific for specific analytes.

Other combinations and permutations will be apparent to one of skill in the art.

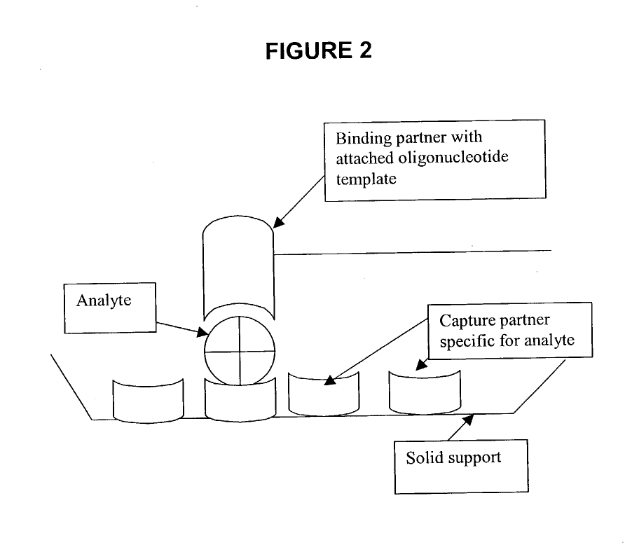

The analyte (or analyte-binding partner complex) may be free in solution, or, in other embodiments, immobilized on a surface, e.g., as part of an array as discussed herein (see FIGS. 2, 4, and 8). Analyte(s) (or analyte-binding partner complex(es)) may be immobilized on a surface (substrate) fabricated from a material such as paper, glass, plastic, polypropylene, nylon, polyacrylamide, nitrocellulose, polystyrene, silicon, metal, and optical fiber. Alternatively, analyte(s) (or analyte-binding partner complex(es)) may be immobilized on the surface (substrate) in a two-dimensional configuration or a three-dimensional configuration comprising pins, rods, fibers, tapes, threads, beads, particles, microtiter wells, capillaries, and/or cylinders. The analyte may be attached to a solid surface . The attachment may be covalent or non covalent. Means of attachment of analyte to solid surface are well-known in the art. See, e.g., U.S. Pat. Nos. 6,309,843; 6,306,365; 6,280,935; 6,087,103 (and methods discussed therein).

Typical analytes may include, but are not limited to proteins, polypeptides and peptides, nucleic acid molecules or segments thereof, lipids, carbohydrates, supramolecular assemblies, organelles, cells, microorganisms and fragments and products thereof, organic molecules, inorganic molecules, or any substance for which one or more attachment sites for binding partner(s) naturally exist or can be developed.

In some embodiments of the invention the analyte is a protein, polypeptide, or peptide. In some of these embodiments, the analyte is a toxin. In some embodiments the analyte is a member of the Botulinum toxin (BoNT) family. The BoNT toxin that serves as an analyte in these embodiments may be present in any amount, including a single molecule. Strains of C. botulinum produce seven different BoNT, with toxin types A, B, E, and F being the main toxins that affect humans. The toxin consists of disulfide linked heavy and light chain, with three major domains. The receptor binding site is at the carboxy terminus of the heavy chain (HC or C-fragment). The binding domain mediates attachment to specific receptors on the presynaptic side of the synapse (Haberman and Dryer (1986) Curr. Topics Microbiol. Immunol. 129: 93-179). The N-terminus of the heavy chain (HN) is a channel-forming domain, which permits the light chain to cross the membrane of the endocytic vesicle. The light chain is a zinc protease, which cleaves one of several proteins on the synaptosomal complex.

2. Binding Partners

Binding partners of the invention include binding partners that bind to the analyte as well as intermediate binding partners that bind to other binding partners. Some intermediate binding partners bind both to other binding partner(s) and to analyte.

The binding partner for the analyte may be any moiety that is capable of binding to the analyte with a desired degree of specificity. As described above, a binding partner may be specific for a single analyte, for multiple analytes in association (such as multisubunit analyte), or for a class of analytes, and/or an analyte may have sites for binding more than one binding partner.

Examples of analyte-binding partner pairs, as well as intermediate binding partner-binding partner pairs include, but are not limited to, receptor ligand, antibody-antigen, two or more antibodies binding to one or more antigens, enzyme-substrate, enzyme-inhibitor, enzyme-cofactor, nucleic acid-probe, subunits of a multi subunit entity, and the like. In all of these examples, as well as others that will be apparent to one of skill in the art, either one of the members of the pair may be binding partner or may be analyte.

In some embodiments, a binding partner may be an antibody, antibody fragment, or antibody derivative specific for an analyte. An antibody binding partner may be polyclonal or monoclonal, and may be human, non-human, chimeric, and/or humanized.

An antibody fragment is a fragment which contains the binding region of the antibody. Such fragments may be Fab-type fragments which are defined as fragments devoid of the Fc portion, e.g., Fab, Fab′ and F(ab′)₂ fragments, or may be “half-molecule” fragments obtained by reductive cleavage of the disulfide bonds connecting the heavy chain components of the intact antibody.

An antibody derivative is an artificial construct that is derived from the amino acid sequences of an antibody or antibodies and that retains the antigen specificity of the original antibody or antibodies. Two or more antibody derivatives may be combined in a single antibody derivative binding partner. Examples of antibody derivatives include, but are not limited to, humanized antibodies, chimeric antibodies, single chain fragment variable fragments (ScFv) and exocyclic peptide-based complementarity determining region (CDR) subunits. The availability of large ScFv and cyclic CDR libraries allows the creation of an antibody derivative binding partner for virtually any molecule. See Zhang et al. (2001) PNAS, 98: 5497-5502; Scott et al. (1999) PNAS 96:13638-13643; Barth et al. (2000) J. Mol. Biol. 301: 751-757. Other examples of antibody derivatives well-known in the art include conjugates.

As described above, the binding partner may be a “universal” binding partner that binds to a multiplicity of different analytes, such as an anti-IgG antibody.

3. Oligonucleotide Template

At least one binding partner that binds directly or indirectly (e.g., via intermediate binding partner(s)) to the analyte is attached to an oligonucleotide template. A portion of the oligonucleotide template is amplified through composite primer-based, single primer isothermal amplification (SPIA™) primer extension, as described below. As used herein, a “portion” of the oligonucleotide template indicates at least a portion.

The oligonucleotide template includes a single stranded (ss) polynucleotide, such as DNA, sequence. For simplicity, DNA is exemplified herein. However, as this disclosure and the definition of oligonucleotide make clear, other nucleic acid embodiments are contemplated and included in the invention. The DNA sequence may include non-standard nucleotides as well as standard nucleotides. The DNA sequence contains at least one primer binding region that is complementary to a composite primer (discussed below) and also contains at least one primer extension region, onto which the primer is extended by a DNA polymerase. The DNA sequence may be flanked on its 3′ and/or 5′ ends by other, non-DNA components such as RNA or PNA, or by non-nucleic acid components such as peptide sequences. The length of the ssDNA portion of the oligonucleotide template is, at least 25, at least 30, or at least 40, or at least 50, or at least 70, or at least 100, or at least 200, or at least 400 nucleotides. The maximum length of the ssDNA portion is determined by the sequences that are included for composite primer binding, primer extension, termination, and/or other sequences that may optionally be added in order to improve the performance and/or efficiency of the amplification and detection of the template, including, but not limited to, sequences designed to bind to probe oligonucleotides, or sequences designed to bind to capture (immobilized or non-immobilized) oligonucleotides.

The oligonucleotide template is attached to the binding partner, either by its 5′ end or its 3′ end, although it may be attached near the 5′ or 3′ end. Methods of attaching a template oligonucleotide to binding partners are known in the art. See, e.g. U.S. Pat. Nos. 6,309,843; 6,306,365; 6,280,935; 6,087,103 (and methods discussed therein). The attachment may be covalent or noncovalent. An example of a noncovalent attachment is an avidin-biotin interaction; thus, the binding partner may be conjugated to streptavidin and the oligonucleotide template may be conjugated to biotin, or bis-biotin may be employed. Another example is his tags binding to chelated metals (see, e.g., Janknecht, et al., 1991, Proc. Nat. Acad. Sci. USA, 88:8972-8976). These methods are known in the art. See, e.g., U.S. Pat. No. 6,153,442. In some embodiments, a tether or linker moiety may be employed between the oligonucleotide template and the binding partner. Such tethers and linkers are well-known in the art.

As stated, at least a portion of the oligonucleotide template serves as a primer extension template. This portion, as well as, generally, a portion of the primer and, optionally, a portion of a TSO or other blocker, serves as a basis for the formation of amplification product. Thus amplification product, discussed in more detail below, is complementary to these combined portions. The sequences of these portions may be chosen so that the amplification product has various desirable characteristics. Thus, the portion of the oligonucleotide template that serves as a primer extension template may contain sequences that, alone or in combination with sequences of these other portions, produces amplification products that hybridize to an oligonucleotide attached to a solid support for capture, and/or that hybridize to a labeled oligonucleotide for detection.

The oligonucleotide template may be synthesized or produced by methods well-known to those of skill in the art, such as recombinant methods.

B. Binding

The analyte(s) and the binding-partner(s) are contacted under conditions that allow binding. Such conditions depend on the nature of the analyte and the binding partners and are well-known in the art. See, e.g., U.S. Pat. Nos. 6,083,689; 5,985,548; 5,854,033; 5,665,539; 5,849,478; 6,255,060; 6,183,960; 5,328,985; 6,210,884; and Sano et al. Science(1992) 258: 120-122; Huang et al. Lett Appl. Microbiol. (2001) 32:321-325; and Zhang et al. PNAS USA (2002) 98: 5497-5502. In some embodiments, the binding of the binding partner to the analyte, or to other binding partner(s), is non-covalent, e.g., in most ligand-receptor binding. In some embodiments the binding may be covalent, e.g., when the analyte is an enzyme and the binding partner is an inhibitor that acts by forming a covalent bond at the active site.

The interaction to be detected or quantified may be carried out using a solid surface, when analyte is immobilized, or in solution. The interaction of the analyte and binding partners, either directly or indirectly (e.g., in a sandwich assay), results in formation of a complex comprising the oligonucleotide template. One or more complexes may be formed simultaneously, each comprising one or more oligonucleotide templates. The detection of the association of specific target/reporter oligonucleotide in the complex may be carried out by separation of the complexes from the non-reacted labeled binding partner, exposing the separated components to conditions which result in generation of multiple copies of oligonucleotide molecules complementary to the target/reporter and detection and, optionally, quantification of the multiple copies. The generation of the multiple copies is carried out by combining the separated components with composite primer of the invention, and amplification reagents.

II. Separation of Bound from Unbound Binding Partner

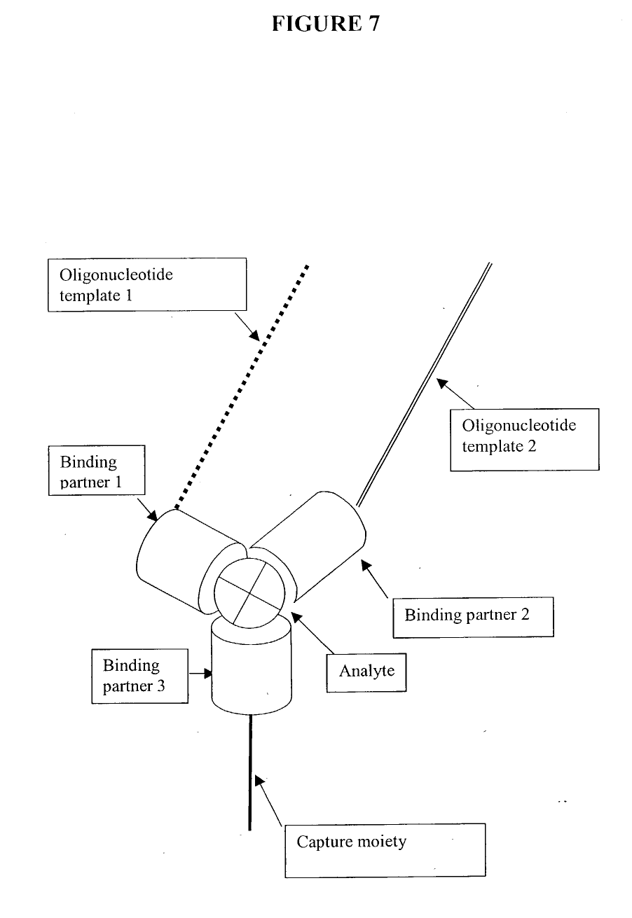

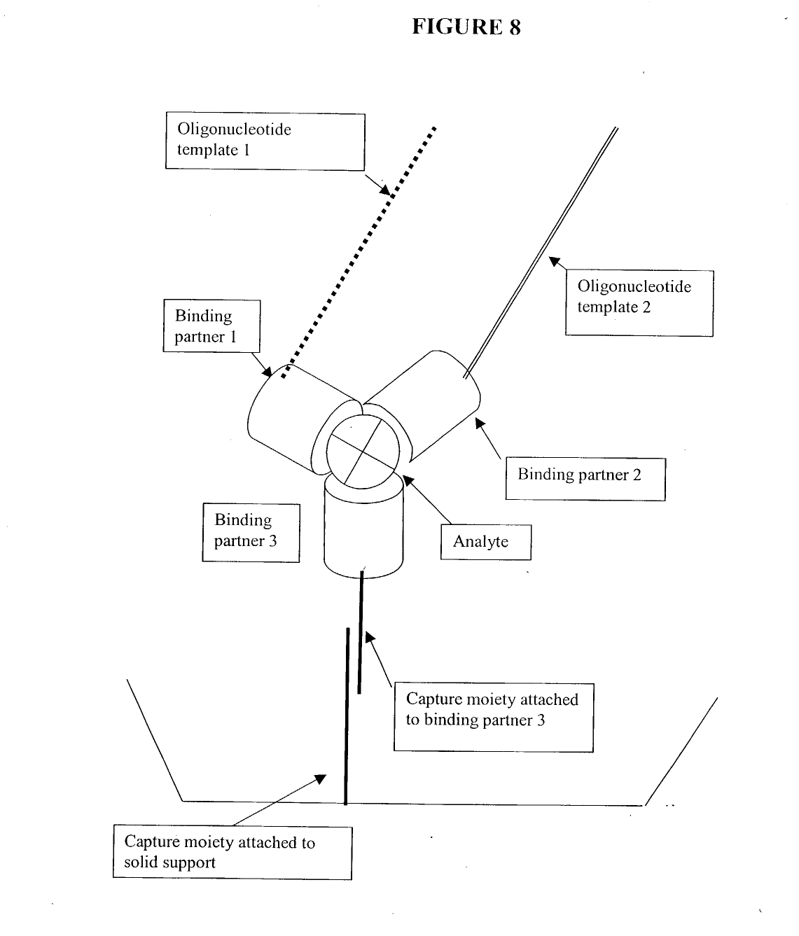

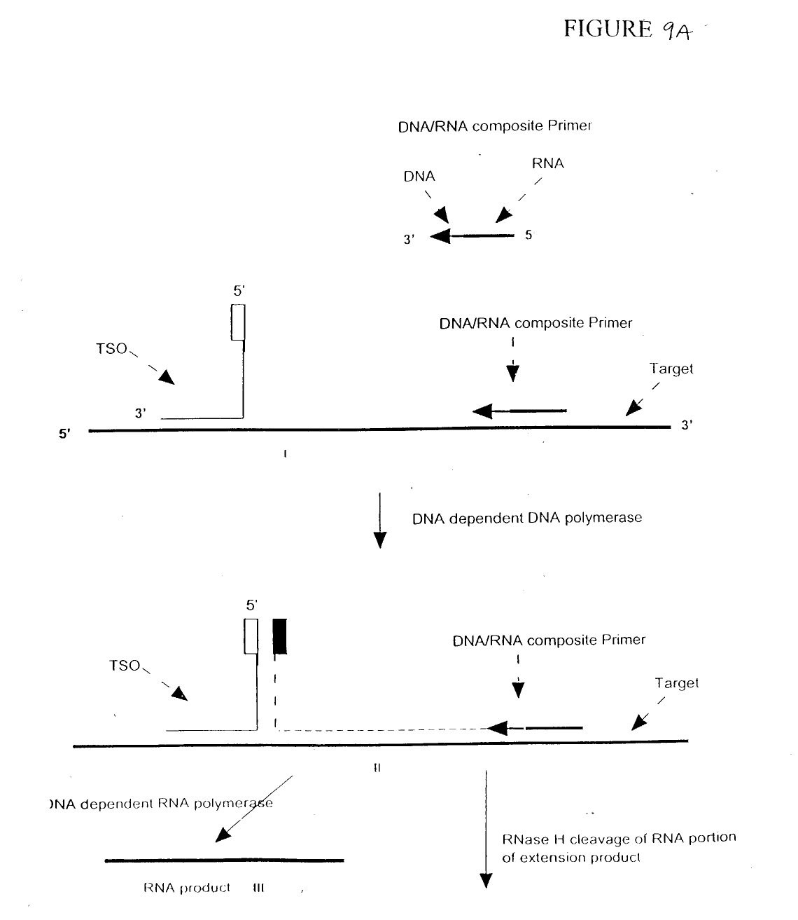

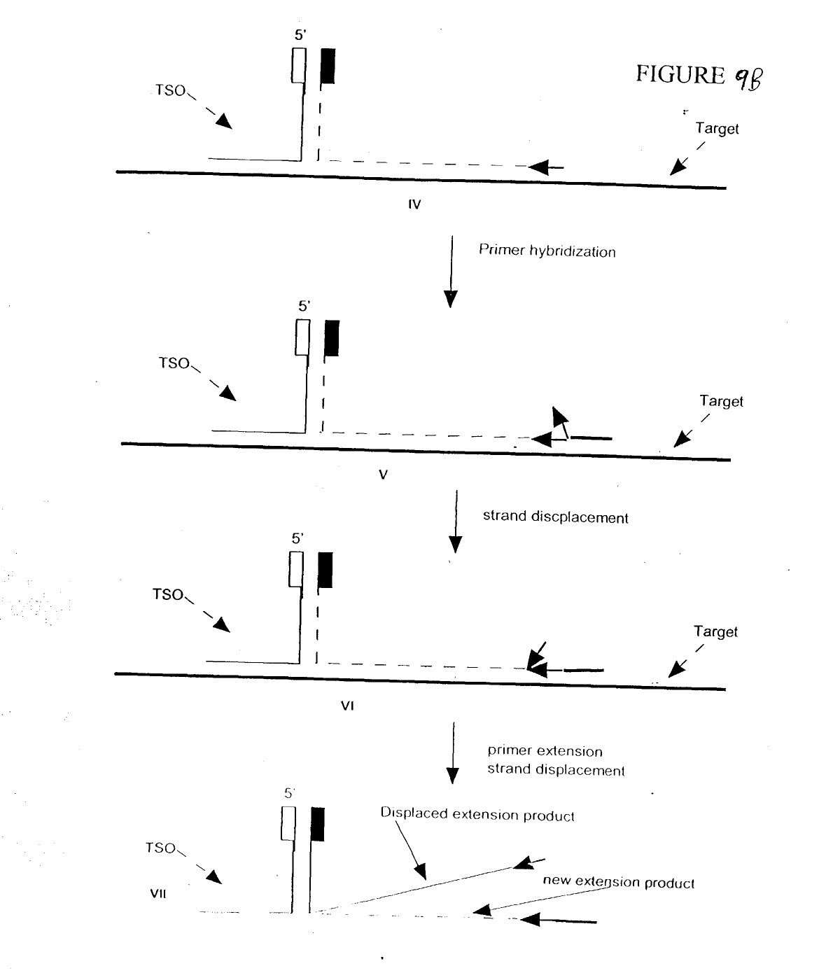

The binding partner-analyte complex may be separated (removed) from unbound binding partner(s) by any suitable means. It is understood that, for purposes of this invention, removal need not be complete and absolute removal, but removal or separation sufficient to permit assessment of presence or absence of analyte (i.e., without significant interference or “noise” arising from amplification of oligonucleotide template attached to unbound binding partner). Such means are well-known in the art. See, e.g., U.S. Pat. Nos. 6,083,689; 5,985,548; 5,854,033; 5,665,539; 5,849,478; 6,255,060; 6,183,960; 5,328,985; 6,210,884; and Sano et al. (1992) Science 258: 120-122; Huang et al. (2001) Lett Appl. Microbiol. 32:321-325; and Zhang et al. (2002) Proc. Natl. Acad. Sci. USA 98: 5497-5502. FIGS. 7 and 8 illustrate an exemplary possibility for separation of analyte-binding partner complex from unbound binding partner. Analyte binds to binding partner(s) that is/are attached to oligonucleotide template(s) for amplification (FIG. 7 shows an analyte bound to two such binding partners, but it will be understood that one such binding partner, or more than two such binding partners, may bind to the analyte). In addition, a binding partner that is attached to a capture moiety binds to the analyte. The capture moiety may be any structure that binds to another structure, e.g., an oligonucleotide that binds to its complementary oligonucleotide, or one member of a streptavidin/biotin pair. Thus, in this example, the analyte-binding partner complex includes binding partner(s) attached to capture moiety(ies), and other binding partner(s) attached to oligonucleotide template(s) for amplification. The analyte-binding partner complex binds to a solid support via interaction of the capture moiety attached to a binding partner in the complex with a capture moiety on the solid support (FIG. 8). For example, the capture moiety on the solid support may be the complementary oligonucleotide to an oligonucleotide attached to binding partner in the analyte-binding partner complex. Unbound binding partner may be removed by, e.g., washing.

Alternatively, bound and unbound binding partner need not be separated. In these embodiments, amplification methods are such that only, or substantially only, bound binding partner is subject to amplification and/or detection. As described above, in one embodiment the methods of the invention require a separation of the analyte-binding partner-oligonucleotide conjugate complex from uncomplexed conjugate. Otherwise, it is difficult to distinguish signal from the complex and from excess free binding partner conjugate. Most assays are also described as heterogeneous because they normally employ a two-phase, heterogeneous system for the separation step. Homogeneous assays occurring in a single phase without any separation are widely recognized as preferable whenever possible because they require less manipulation of the reaction mixture-typically only a series of reagent additions rather than the transfer and washing steps common to heterogeneous methods. Thus, they are faster, require less labor to perform, and are easier to automate. However, homogeneous assays are limited in many cases because the distinction between signal from bound and unbound labels is rarely complete. Consequently, a homogeneous assay with a given signal level will typically have a higher background signal and thus lower sensitivity than a similar heterogeneous assay.

In some embodiments, signal may be preferentially generated from an analyte-binding pair complex by taking advantage of the well-known “proximity effect”: two or more functional groups, molecules, or even proteins will react more rapidly with each other when other structures hold them in close proximity to each other. The origin of this effect is well understood, arising from decreases in translational and rotational entropy on binding, and examples are known in a variety of fields. A related phenomenon, the “channeling” effect, has been exploited in the design of high-sensitivity homogeneous assays.

The present invention offers several possible ways to exploit the proximity effect by bringing reactants together. In particular, either polymerase or RNase is attached to a second binding partner for the analyte. Formation of a sandwich complex brings together a high local concentration of the enzyme and the oligonucleotide-binding partner-primer complex, allowing the enzyme to act more rapidly in either extending or digesting the primer compared to the same enzyme diffusing freely through solution. Consequently, this sandwich complex produces more extension product per unit time than is generated by uncomplexed product in solution. The rate enhancements depend on many factors, most notably the concentrations of the reagents in solution compared to the “local concentration” in the complex, but they can be several orders of magnitude.

This proximity effect might also be useful in conjunction with a SPIA™-enhanced heterogeneous assay (i.e., assay utilizing a separation step). In many high-sensitivity assays, detection is actually limited by background signal generated by label nonspecifically bound to surfaces. This is likely to be the case in particular with assays such as immuno-PCR where any nonspecifically bound material can be exponentially amplified. However, if a sandwich complex such as described above is formed on a surface, signal is generated only from that complex, but not from other binding partner conjugates bound nonspecifically to the surface. Thus it is possible to obtain both very high signal from the SPIATM enhancement and very low background from a combination of washing (separation) and the proximity effect.

III. Amplification of Oligonucleotide Template

A. Components, Reaction Conditions, and Procedures

1. Composite Primer

The primer binding region of the oligonucleotide template hybridizes to a composite primer under suitable conditions, and the composite primer is extended along the primer extension template region of the oligonucleotide template, and optionally along other components such as a template switch oligonucleotide (see below), by a DNA polymerase to produce a primer extension product. The composite primer is composed of RNA and DNA portions. The composite design of the primer is important for subsequent displacement of the primer extension product by binding of a new (additional) composite primer and the extension of the new primer by the polymerase. In addition, cleavage of RNA of the primer extension product leads to generation of amplification product which is not a substrate for amplification by the composite primer, as described below. Composite primers, as well as the method to amplify (SPIA™) are described in, e.g., U.S. Pat. No. 6,251,639.

Composite primers for use in the methods and compositions of the present invention comprise at least one RNA portion that is capable of (a) binding (hybridizing) to a sequence of the primer binding region of the oligonucleotide template independent of hybridization of the DNA portion(s) to a sequence on the primer binding region of the oligonucleotide template; and (b) being cleaved with a ribonuclease when hybridized to the oligonucleotide template DNA. The composite primers bind to the primer binding region of the oligonucleotide template to form a partial heteroduplex in which only RNA of the primer is cleaved upon contact with a ribonuclease such as RNase H, while the oligonucleotide template strand remains intact, thus enabling annealing of another composite primer.

The composite primers also comprise a 3′ DNA portion that is capable of hybridization to a sequence of the primer binding region of the oligonucleotide template such that its hybridization to the oligonucleotide template is favored over that of the nucleic acid strand that is displaced from the oligonucleotide template by the DNA polymerase. Such primers can be rationally designed based on well known factors that influence nucleic acid binding affinity, such as sequence length and/or identity, as well as hybridization conditions. In a preferred embodiment, hybridization of the 3′ DNA portion of the composite primer to its complementary sequence in the primer binding region of the oligonucleotide template is favored over the hybridization of the homologous sequence in the 5′ end of the displaced strand to the oligonucleotide template.

Generation of primers suitable for extension by polymerization is well known in the art, such as described in PCT Pub. No. WO99/42618 (and references cited therein). The composite primer comprises a combination of RNA and DNA (see definition above), with the 3′-end nucleotide being a nucleotide suitable for nucleic acid extension. The 3′-end nucleotide can be any nucleotide or analog that when present in a primer, is extendable by a DNA polymerase. Generally, the 3′-end nucleotide has a 3′-OH. Suitable primers include those that comprise at least one portion of RNA and at least one portion of DNA.

Composite primers can comprise a 5′-RNA portion and a 3′-DNA portion (in which the RNA portion is adjacent to the 3′-DNA portion); or 5′- and 3′-DNA portions with an intervening RNA portion. Accordingly, in one embodiment, the composite primer comprises a 5′ RNA portion and a 3′-DNA portion, preferably wherein the RNA portion is adjacent to the 3′-DNA portion. In another embodiment, the composite primer comprises 5′- and 3′-DNA portions with at least one intervening RNA portion (i.e., an RNA portion between the two DNA portions). In yet another embodiment, the composite primer of the present invention comprises a3′-DNA portion and at least one intervening RNA portion (i.e., an RNA portion between DNA portions).

The length of an RNA portion in a composite primer comprising a 3′-DNA portion and an RNA portion can be preferably from about 1 to about 25, more preferably from about 3 to about 20, even more preferably from about 4 to about 15, and most preferably from about 5 to about 10 nucleotides. In some embodiments of a composite primer comprising a 3′-DNA portion and an RNA portion, an RNA portion can be at least about any of 1, 2, 3, 4, 5 nucleotides, with an upper limit of about any of 10, 15, 20, 25, 30 nucleotides.

The length of the 5′-RNA portion in a composite primer comprising a 5′-RNA portion and a 3′-DNA portion can be preferably from about 3 to about 25 nucleotides, more preferably from about 5 to about 20 nucleotides, even more preferably from about 7 to about 18 nucleotides, preferably from about 8 to about 17 nucleotides, and most preferably from about 10 to about 15 nucleotides. In other embodiments of a composite primer comprising a 5′-RNA portion and a 3′-DNA portion, the 5′-RNA portion can be at least about any of 3, 5, 7, 8, 10 nucleotides, with an upper limit of about any of 15, 17, 18, 20 nucleotides.

In embodiments of a composite primer comprising a 5′-RNA portion and a 3′-DNA portion further comprising non-5′-RNA portion(s), a non-5′-RNA portion can be preferably from about 1 to about 7 nucleotides, more preferably from about 2 to about 6 nucleotides, and most preferably from about 3 to about 5 nucleotides. In certain embodiments of a composite primer comprising a 5′-RNA portion and a 3′-DNA portion further comprising non-5′-RNA portion(s), a non-5′-RNA portion can be at least about any of 1, 2, 3, 5, with an upper limit of about any of 5, 6, 7, 10 nucleotides.

In embodiments of a composite primer comprising a 5′-RNA portion and a 3′-DNA portion, in which the 5′-RNA portion is adjacent to the 3′-DNA portion, the length of the 5′-RNA portion can be preferably from about 3 to about 25 nucleotides, more preferably from about 5 to about 20 nucleotides, even more preferably from about 7 to about 18 nucleotides, preferably from about 8 to about 17 nucleotides, and most preferably from about 10 to about 15 nucleotides. In certain embodiments of a composite primer comprising a 5′-RNA portion and a 3′-DNA portion, in which the 5′-RNA portion is adjacent to the 3′-DNA portion, the 5′-RNA portion can be at least about any of 3, 5, 7, 8, 10 nucleotides, with an upper limit of about any of 15, 17, 18, 20 or nucleotides.

The length of an intervening RNA portion in a composite primer comprising 5′- and 3′-DNA portions with at least one intervening RNA portion can be preferably from about 1 to about 7 nucleotides, more preferably from about 2 to about 6 nucleotides, and most preferably from about 3 to about 5 nucleotides. In some embodiments of a composite primer comprising 5′- and 3′-DNA portions with at least one intervening RNA portion, an intervening RNA portion can be at least about any of 1, 2, 3, 5 nucleotides, with an upper limit of about any of 5, 6, 7, 10 nucleotides. The length of an intervening RNA portion in a composite primer comprising a 3′-DNA portion and at least one intervening RNA portion can be preferably from about 1 to about 7 nucleotides, more preferably from about 2 to about 6 nucleotides, and most preferably from about 3 to about 5 nucleotides. In some embodiments of a composite primer comprising a 3′-DNA portion and at least one intervening RNA portion, an intervening RNA portion can be at least about any of 1, 2, 3, 5 nucleotides, with an upper limit of about any of 5, 6, 7, 10 nucleotides. In a composite primer comprising a 3′-DNA portion and at least one intervening RNA portion, further comprising a 5′-RNA portion, the 5′-RNA portion can be preferably from about 3 to about 25 nucleotides, more preferably from about 5 to about 20 nucleotides, even more preferably from about 7 to about 18 nucleotides, preferably from about 8 to about 17 nucleotides, and most preferably from about 10 to about 15 nucleotides. In some embodiments of a composite primer comprising a 3′-DNA portion and at least one intervening RNA portion, further comprising a 5′-RNA portion, the 5′-RNA portion can be at least about any of 3, 5, 7, 8, 10 nucleotides, with an upper limit of about any of 15, 17, 18, 20 nucleotides.

The length of the 3′-DNA portion in a composite primer comprising a 3′-DNA portion and an RNA portion can be preferably from about 1 to about 20, more preferably from about 3 to about 18, even more preferably from about 5 to about 15, and most preferably from about 7 to about 12 nucleotides. In some embodiments of a composite primer comprising a 3′-DNA portion and an RNA portion, the 3′-DNA portion can be at least about any of 1, 3, 5, 7, 10 nucleotides, with an upper limit of about any of 10, 12, 15, 18, 20, 22 nucleotides.

The length of the 3′-DNA portion in a composite primer comprising a 5′-RNA portion and a 3′-DNA portion can be preferably from about 1 to about 20 nucleotides, more preferably from about 3 to about 18 nucleotides, even more preferably from about 5 to about 15 nucleotides, and most preferably from about 7 to about 12 nucleotides. In some embodiments of a composite primer comprising a 5′-RNA portion and a 3′-DNA portion, the 3′ DNA portion can be at least about any of 1, 3, 5, 7, 10 nucleotides, with an upper limit of about any of 10, 12, 15, 18, 20, 22 nucleotides.

In embodiments of a composite primer comprising a 5′-RNA portion and a 3′-DNA portion, further comprising non-3′-DNA portion(s), a non-3′-DNA portion can be preferably from about 1 to about 10 nucleotides, more preferably from about 2 to about 8 nucleotides, and most preferably from about 3 to about 6 nucleotides. In some embodiments of a composite primer comprising a 5′-RNA portion and a 3′-DNA portion, further comprising non-3′-DNA portion(s), a non-3′-DNA portion can be at least about any of 1, 2, 3, 5 nucleotides, with an upper limit of about any of 6, 8, 10, 12 nucleotides.

In embodiments of a composite primer comprising a 5′-RNA portion and a 3′-DNA portion in which the 5′-RNA portion is adjacent to the 3′-DNA portion, the length of the 3′-DNA portion can be preferably from about 1 to about 20 nucleotides, more preferably from about 3 to about 18 nucleotides, even more preferably from about 5 to about 15 nucleotides, and most preferably from about 7 to about 12 nucleotides. In certain embodiments of the primer comprising a 5′-RNA portion and a 3′-DNA portion in which the 5′-RNA portion is adjacent to the 3′-DNA portion, the 3′-DNA portion can be at least about any of 1, 3, 5, 7, 10 nucleotides, with an upper limit of about any of 10, 12, 15, 18, 20, 22 nucleotides.

The length of a non-3′-DNA portion in a composite primer comprising 5′- and 3′-DNA portions with at least one intervening RNA portion can be preferably from about 1 to about 10 nucleotides, more preferably from about 2 to about 8 nucleotides, and most preferably from about 3 to about 6 nucleotides. In some embodiments of a primer comprising 5′- and 3′-DNA portions with at least one intervening RNA portion, a non-3′-DNA portion can be at least about any of 1, 2, 3, 5 nucleotides, with an upper limit of about any of 6, 8, 10, 12 nucleotides.

The length of the 3′-DNA portion in a composite primer comprising 5′- and 3′-DNA portions with at least one intervening RNA portion can be preferably from about 1 to about 20 nucleotides, more preferably from about 3 to about 18 nucleotides, even more preferably from about 5 to about 15 nucleotides, and most preferably from about 7 to about 12 nucleotides. In some embodiments of a composite primer comprising 5′- and 3′-DNA portions with at least one intervening RNA portion, the 3′-DNA portion can be at least about any of 1, 3, 5, 7, 10 nucleotides, with an upper limit of about any of 10, 12, 15, 18, 20, 22 nucleotides.

The length of a non-3′-DNA portion (i.e., any DNA portion other than the 3′-DNA portion) in a composite primer comprising a 3′-DNA portion and at least one intervening RNA portion can be preferably from about 1 to about 10 nucleotides, more preferably from about 2 to about 8 nucleotides, and most preferably from about 3 to about 6 nucleotides. In some embodiments of a composite primer comprising a 3′-DNA portion and at least one intervening RNA portion, a non-3′-DNA portion can be at least about any of 1, 3, 5, 7, 10 nucleotides, with an upper limit of about any of 6, 8, 10, 12 nucleotides. The length of the 3′-DNA portion in a composite primer comprising a 3′-DNA portion and at least one intervening RNA portion can be preferably from about 1 to about 20 nucleotides, more preferably from about 3 to about 18 nucleotides, even more preferably from about 5 to about 15 nucleotides, and most preferably from about 7 to about 12 nucleotides. In some embodiments of a composite primer comprising a 3′-DNA portion and at least one intervening RNA portion, the 3′-DNA portion can be at least about any of 1, 3, 5, 7, 10 nucleotides, with an upper limit of about any of 10, 12, 15, 18, 20, 22 nucleotides. It is understood that the lengths for the various portions can be greater or less, as appropriate under the reaction conditions of the methods of this invention.

In some embodiments, the 5′-DNA portion of a composite primer includes the 5′-most nucleotide of the primer. In some embodiments, the 5′-RNA portion of a composite primer includes the 5′ most nucleotide of the primer. In other embodiments, the 3′-DNA portion of a composite primer includes the 3′ most nucleotide of the primer. In other embodiments, the 3′-DNA portion is adjacent to the 5′-RNA portion and includes the 3′ most nucleotide of the primer (and the 5′-RNA portion includes the 5′ most nucleotide of the primer).

The total length of the composite primer can be preferably from about 10 to about 40 nucleotides, more preferably from about 15 to about 30 nucleotides, and most preferably from about 20 to about 25 nucleotides. In some embodiments, the length can be at least about any of 10, 15, 20, 25 nucleotides, with an upper limit of about any of 25, 30, 40, 50 nucleotides. It is understood that the length can be greater or less, as appropriate under the reaction conditions of the methods of this invention. In some embodiments the RNA portion of the composite primer consists of, for example, about 7 to about 20 nucleotides and the DNA portion of the composite primer consists of, for example, about 5 to about 20 nucleotides. In other embodiments, the RNA portion of the composite primer consists of, for example, about 10 to about 20 nucleotides and the DNA portion of the composite primer consists of, for example, about 7 to about 20 nucleotides.

To achieve hybridization (which, as is well known and understood in the art, depends on other factors such as, for example, ionic strength and temperature), composite primers for use in the methods and compositions of the present invention are preferably of at least about 60%, more preferably at least about 75%, even more preferably at least about 90%, and most preferably at least about 95% complementarity to the primer binding region of the oligonucleotide template. The individual DNA and RNA portions of the composite primers are preferably of at least about 60%, more preferably at least about 75%, even more preferably at least about 90%, and most preferably at least about 95% complementarity to the primer binding region of the oligonucleotide template.

The hybridization conditions chosen depend on a variety of factors known in the art, for example the length and type (e.g., RNA, DNA, PNA) of primer and primer binding region of the oligonucleotide template. General parameters for specific (i.e., stringent) hybridization conditions for nucleic acids are described in Sambrook (1989), supra, and in Ausubel (1987), supra. Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press San Diego, Calif. For a given set of reaction conditions, the ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by any one or more of the following: elevating the temperature, increasing the ratio of cosolvents, lowering the salt concentration, and the like.

One factor in designing and constructing primers is the free energy parameters of hybridization of given sequences under a given set of hybridization conditions. The free energy parameters for the formation of a given hybrid may be calculated by methods known in the art (see, e.g., Tinoco et al. Nature (1973) 246:40-41. and Freier et al., Proc. Natl. Acad. Sci. USA (1986) 83:9373-9377; computer programs, e.g., Oligo Primer Analysis Software from Molecular Biology Insight, and references therein), and it is possible to predict, for a given oligonucleotide template, primer sequences for which the required free energy changes for formation of various complexes will be met.

One of skill in the art will understand that other factors affect nucleic acid hybridization affinities. For example, any and all of the guanosine-cytosine content of the primer-target and primer-primer duplexes, minor groove binders, O-methylation or other modification of nucleotides, temperature, and salt are potentially important factors in constructing primers with the requisite differences in binding energies.

As described herein, one or more composite primers may be used in a reaction.

2. A Polynucleotide Comprising a Termination Polynucleotide Sequence

Although it is not a necessary component of the invention, in some embodiments of the methods of the present invention, especially if transcription-based amplification is used, a polynucleotide comprising a termination sequence is optionally included, examples of which are provided. A “propromoter,” or “propromoter sequence” is a sequence that is designed for formation of a double stranded promoter of an RNA polymerase. In some embodiments, the polynucleotide is a TSO which contains a propromoter sequence, as discussed in the section describing TSO's. Such polynucleotides are described in, e.g., U.S. Pat. No. 6,251,639.

a. Template Switch Oligonucleotide

A second oligonucleotide that can optionally, though not necessarily, be used in the amplification methods of the invention is a template switch oligonucleotide (TSO). In one embodiment, the TSO functions as a termination sequence. In another embodiment, the TSO functions as a termination sequence and provides a propromoter sequence.

Previously described amplification methods based on template switch oligonucleotide were restricted in the concentration of this oligonucleotide due to inhibition of hybridization of the second primer, or the second hybridization step of the same primer when the method is designed to utilize a single primer species. The methods of the invention are free of this limitation. In contrast to previously described methods using TSOs, the template switch oligonucleotide can be used at high concentration for amplification according to the methods of the present invention. This feature ensures efficient hybridization of the TSO to the oligonucleotide template, and maximizes the yield of the tri molecular complex, the substrate for primer extension and template switch. An additional attribute of this feature is the efficient hybridization of the displaced primer extension product to the template switch oligonucleotide to form a substrate for the RNA polymerase, as described.

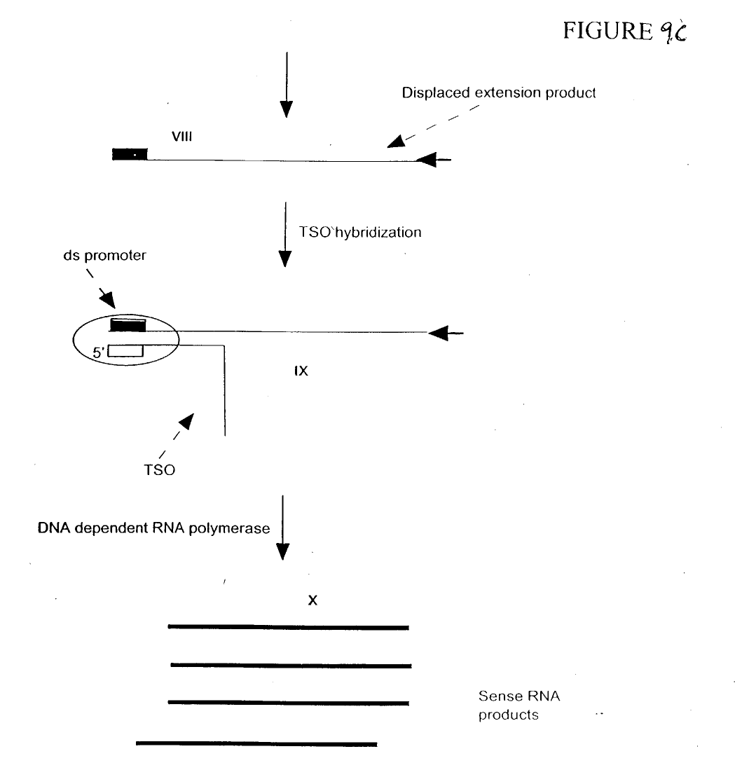

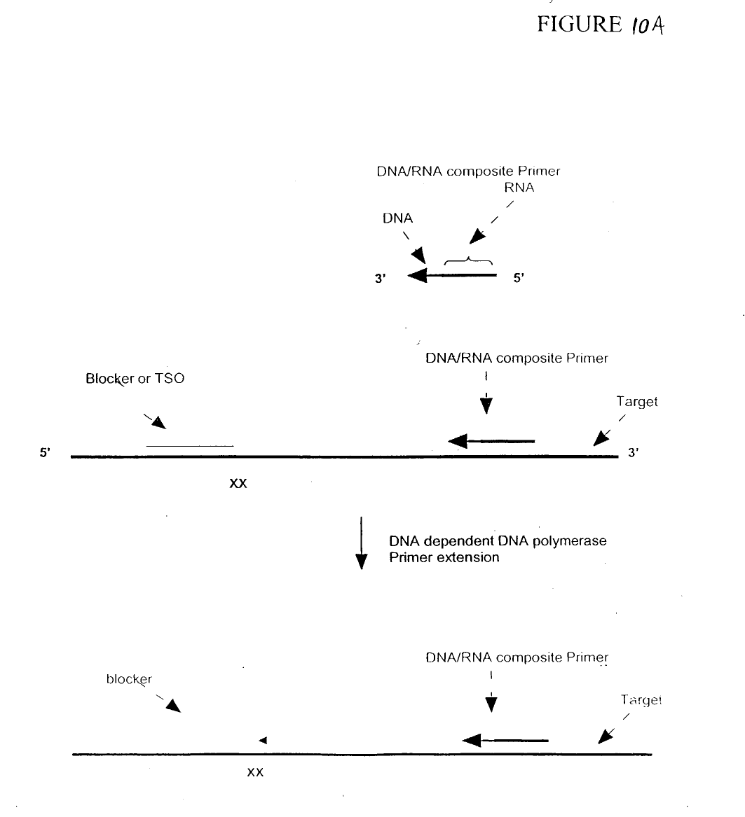

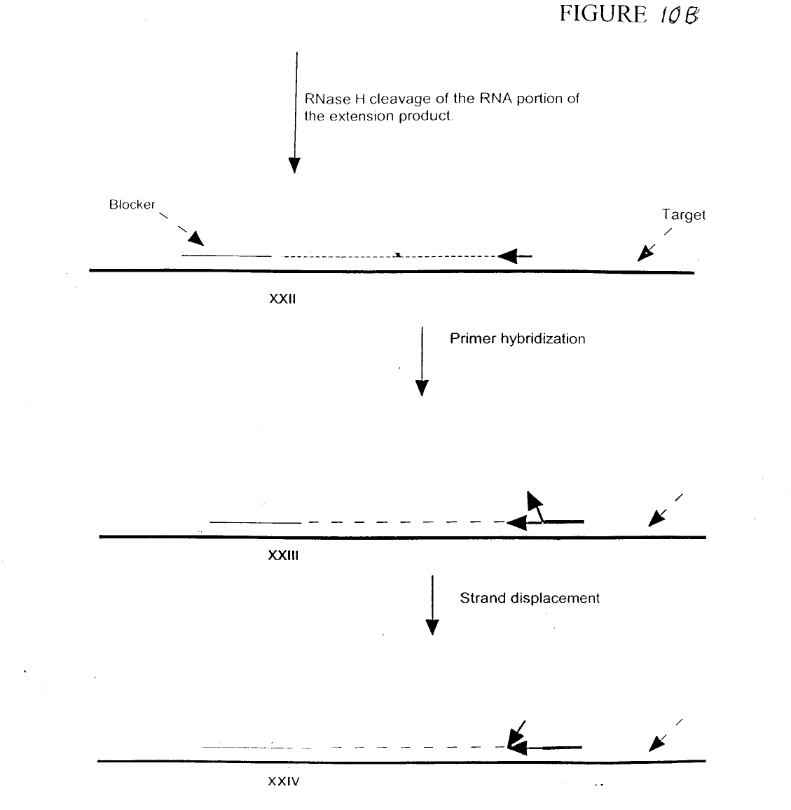

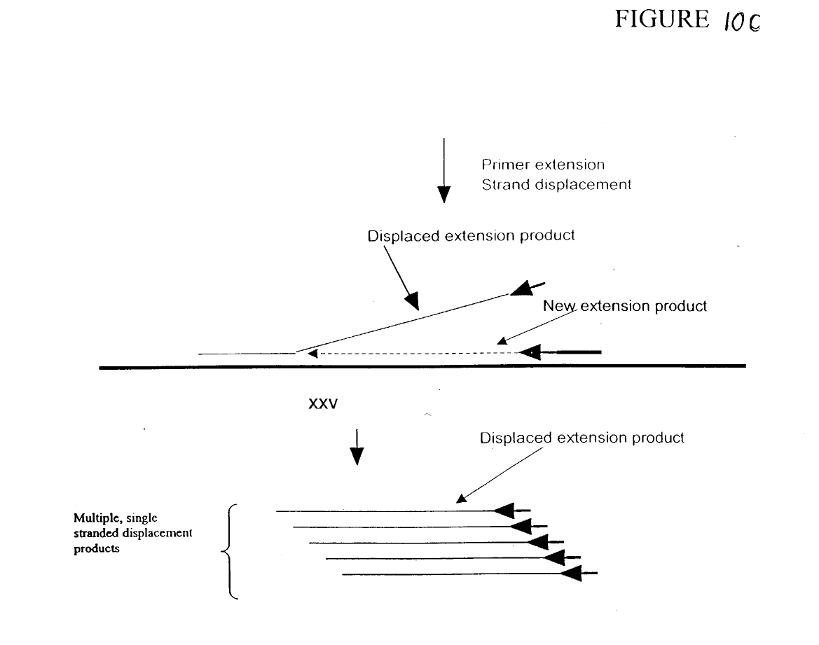

A TSO comprises a 3′ portion that can hybridize to the oligonucleotide template and a 5′ portion which is designed for strand switch during polymerization see FIGS. 9A-C. Design of a TSO that would effect strand switch is known in the art, such as was previously described in Patel et al., Proc. Nat'l. Acad. Sci. USA 1996, 93:2969-2974. The 3′ portion hybridizes to the oligonucleotide template at a location 5′ to the position or region in the oligonucleotide template that is complementary to the 3′ end of the primer extension product prior to switching template from the oligonucleotide template to the unhybridized portion of the TSO (“termination site”).

In one embodiment, strand switch is promoted by the presence of mutually complementary short sequences in the TSO segments immediately 5′ and 3′ to the junction between the hybridized and non-hybridized portions of the TSO. Without intending to be bound by theory, one explanation is that in the event that the primer extension product is extended into the portion of the oligonucleotide template that is hybridized to the TSO (through displacement of the hybridized portion of the TSO), the 3′ end of the primer extension product would comprise a short sequence that can bind to its complementary short sequence in the segment of the TSO immediately adjacent to the junction between the hybridized and non-hybridized portions of the TSO. This increases the efficiency of template switching by increasing the probability that the primer extension product would switch to the TSO tail portion as a template. The length of the short complementary sequences is preferably from about 3 to about 20 nucleotides, more preferably from about 5 to about 15 nucleotides, and most preferably from about 7 to about 10 nucleotides. In some embodiments, length is at least about any of 1, 3, 5, 7, 10 nucleotides, with an upper limit of about any of 10, 15, 20, 25 nucleotides. It is understood that the length can be greater or less, as appropriate under the reaction conditions of the methods of this invention.

In some embodiments, the 5′ portion of the TSO comprises a sequence (hereinafter “propromoter sequence”),This embodiment of the TSO would function both as a termination sequence and to provide a promoter template. In this embodiment, the propromoter sequence of the TSO serves as a template for incorporation of a propromoter sequence (generally complementary to the propromoter sequence of the template TSO) into the primer extension product. Subsequent hybridization of a TSO comprising a propromoter sequence that is hybridizable (e.g., that hybridizes) to the propromoter sequence of the primer extension product results in formation of a double stranded promoter capable of effecting transcription by a suitable RNA polymerase. Promoter sequences that allow transcription of a template DNA are known in the art, as are methods of obtaining and/or making them. Preferably, the promoter sequence is selected to provide optimal transcriptional activity of the particular RNA polymerase used. Criteria for such selection, i.e., a particular promoter sequence particularly favored by a particular RNA polymerase, are also known in the art. For example, the sequences of the promoters for transcription by T7 DNA dependent RNA polymerase and SP6 are known in the art. The promoter sequence can be from a prokaryotic or eukaryotic source. In one embodiment, the promoter sequence is adjacent to a sequence that is designed to provide for enhanced, or more optimal, transcription by the RNA polymerase used. In some embodiments, the sequence is not related (i.e., it does not substantially hybridize) to the oligonucleotide template. More optimal transcription occurs when transcriptional activity of the polymerase from a promoter that is operatively linked to said sequence is greater than from a promoter that is not so linked. The sequence requirements for optimal transcription are generally known in the art as previously described for various DNA dependent RNA polymerases, such as in U.S. Pat. Nos. 5,766,849 and 5,654,142.

In a preferred embodiment, a segment of the 3′ portion of the TSO (including the entire 3′ portion that hybridizes to oligonucleotide template) that hybridizes to the oligonucleotide template DNA is attached to the oligonucleotide template DNA such that displacement of the TSO by the polymerase that effects primer extension is substantially, or at least sufficiently, inhibited. Suitable methods for achieving such attachment includes techniques known in the art, such as using a cytosine analog that contains a G-clamp heterocycle modification (described in Flanagan et al., Proc. Natl. Acad. Sci. USA 1999, 96(7):3513-8); and locked nucleic acids (described, e.g., in Kumar et al., Bioorg. Med. Chem Lett. 1998, 8(16):2219-22; and Wahlestedt et al., Proc. Natl. Acad. Sci. USA 2000, 97(10):5633-8). Other suitable methods include using, where appropriate, sequences with a high GC content and/or cross-linking. Any of these methods for obtaining enhanced attachment may be used alone or in combination. Displacement of the TSO is substantially or sufficiently inhibited if the polymerase switches template from the oligonucleotide template to the unhybridized portion of the TSO in at least about 25%, preferably at least about 50%, more preferably at least about 75%, and most preferably at least about 90%, of the events of primer extension. Substantially or sufficiently inhibited TSO displacement can also be empirically indicated if the amplification methods lead to a satisfactory result in terms of amount of the desired product. Generally, under a given set of conditions, the “modified” TSO binds more tightly to template as compared to a TSO not so modified.

The length of the TSO portion that hybridizes to the oligonucleotide template is preferably from about 15 to 50 nucleotides, more preferably from about 20 to 45 nucleotides, and most preferably from about 25 to 40 nucleotides. In other embodiments, the length is at least about any of the following: 10, 15, 20, 25, 30; and less than about any of the following: 35, 40, 45, 50, 55. It is understood that the length can be greater or less, as appropriate under the reaction conditions of the methods of this invention. The complementarity of the TSO portion that hybridizes to the oligonucleotide template is preferably at least about 25%, more preferably at least about 50%, even more preferably at least about 75%, and most preferably at least about 90%, to its intended binding sequence on the oligonucleotide template.

b. Blocker Sequence

In some embodiments, the primer extension termination sequence is provided by an optional blocker sequence. The blocker sequence is a polynucleotide, usually a synthetic polynucleotide, that is single stranded and comprises a sequence that is hybridizable (e.g., that hybridizes), preferably complementary, to a segment of oligonucleotide template sequence 5′ of the position in the oligonucleotide template that is complementary to the 3′ end of the primer extension product (“termination site”). The blocker comprises nucleotides that bind to the target nucleic acid with an affinity, preferably a high affinity, such that the blocker sequence resists displacement by DNA polymerase in the course of primer extension, in preferably more than about 30%, more preferably more than about 50%, even more preferably more than about 75%, and most preferably more than about 90%, of primer extension events. The length and composition of the blocker polynucleotide should be such that excessive random non-specific hybridization is avoided under the conditions of the methods of the present invention. The length of the blocker polynucleotide is preferably from about 3 to about 30 nucleotides, more preferably from about 5 to about 25 nucleotides, even more preferably from about 8 to about 20 nucleotides, and most preferably from about 10 to about 15 nucleotides. In other embodiments, the blocker polynucleotide is at least about any of the following: 3, 5, 8, 10, 15; and less than about any of the following: 20, 25, 30, 35. It is understood that the length can be greater or less as appropriate under the reaction conditions of the methods of this invention. The complementarity of the blocker polynucleotide is preferably at least about 25%, more preferably at least about 50%, even more preferably at least about 75%, and most preferably at least about 90%, to its intended binding sequence on the oligonucleotide template.

In one embodiment, the blocker sequence comprises a segment that hybridizes to the oligonucleotide template such that displacement of the blocker sequence by the polymerase that effects primer extension is substantially, or at least sufficiently, inhibited. Suitable means for achieving such attachment and determining substantial, or sufficient, inhibition of displacement are as described above for TSO used in the methods of the present invention.

In one embodiment, the blocker polynucleotide cannot function efficiently as a primer for nucleic acid extension (i.e., extension from the blocker sequence is reduced, or inhibited). Techniques for blocking the primer function of the blocker polynucleotide include any that prevent addition of nucleotides to the 3′ end of the blocker by a DNA polymerase. Such techniques are known in the art, including, for example, substitution or modification of the 3′ hydroxyl group, or incorporation of a modified nucleotide, such as a dideoxynucleotide, in the 3′-most position of the blocker polynucleotide that is not capable of anchoring addition of nucleotides by a DNA polymerase.

3. Polynucleotide Comprising a Termination Sequence and Further Comprising a Propromoter Sequence

In some embodiments of methods of the invention, an optional termination sequence and a propromoter sequence are provided in a single polynucleotide. The polynucleotide comprises a portion (generally a 3′ portion) that comprises a termination sequence that does not effect template switch under conditions wherein the termination sequence is hybridizable (e.g., hybridizes) to an oligonucleotide template, and a portion (generally a 5′ portion) that comprises a propromoter sequence, wherein the portion that comprises a propromoter sequence generally does not hybridize to the oligonucleotide template (under conditions wherein the portion that comprises a termination sequence hybridizes to the oligonucleotide template). A termination sequence can be designed so as not to effect template switch using techniques known in the art, for example by ensuring that design characteristics that are known to promote template switch (such as described in Patel et al., Proc. Nat'l Acad. Sci. USA 1996, 93:2969-2974) are not present in the polynucleotide. The polynucleotide is hybridizable (e.g., hybridizes) to the sequence of the oligonucleotide template that is in the 5′ direction with respect to the template sequence which is hybridizable (e.g., hybridizes) to the primer. The polynucleotide further comprises a sequence which is hybridizable (e.g., hybridizes) to a complementary sequence of the oligonucleotide template. The sequence that is hybridizable (e.g., hybridizes) to a complementary sequence of the oligonucleotide template may be non-overlapping, overlapping or co-extensive with the termination sequence and/or propromoter sequence of the combination polynucleotide. Generally and preferably the sequence that is hybridizable (e.g., hybridizes) to a complementary sequence of the oligonucleotide template is hybridizable (e.g., hybridizes) to a 3′ portion of the complementary sequence of the oligonucleotide template. Thus, in some embodiments of methods of the invention, a polynucleotide that comprises a portion comprising a termination sequence and a portion comprising a propromoter sequence functions both to effect termination of primer extension and to provide a propromoter sequence in the same amplification reaction. Such polynucleotides are described in, e.g., U.S. Pat. No. 6,251,639.

4. Polynucleotide Comprising a Propromoter and a Region which Hybridizes to a Displaced Primer Extension Product

Some embodiments employ a polynucleotide comprising a propromoter and a region which hybridizes to a displaced primer extension product. In some embodiments, the polynucleotide is a TSO which contains a propromoter sequence, as discussed above. In other embodiments, the propromoter sequence is contained in a PTO, as described below. Such polynucleotides are described in, e.g., U.S. Pat. No. 6,251,639. In some embodiments a propromoter without PTO may be used.

a. Propromoter Template Oligonucleotide

In some embodiments, the methods employ a promoter sequence for transcription which is provided by a propromoter template oligonucleotide (PTO). A PTO for use in the methods and compositions of the present invention is a single-stranded polynucleotide, generally DNA, comprising a propromoter sequence that is designed for formation of a ds promoter of an RNA polymerase, and a portion capable of hybridizing to the 3′ end of the primer extension product. In a preferred embodiment, the propromoter sequence is located in the 5′ portion of the oligonucleotide and the hybridizing sequence is located in the 3′ portion of the oligonucleotide. In one embodiment, and most typically, the promoter and hybridizing sequences are different sequences. In another embodiment, the promoter and hybridizing sequences overlap in sequence identity. In yet another embodiment, the promoter and hybridizing sequences are the same sequence, and thus are in the same location on the PTO. In the embodiments wherein hybridization of the PTO to the primer extension product results in a duplex comprising an overhang (the 5′ end of the PTO that does not hybridize to the displaced primer extension product, typically comprising all or part of the propromoter sequence), DNA polymerase fills in the overhang to create a double stranded promoter capable of effecting transcription by a suitable RNA polymerase.

Promoter sequences that allow transcription of a template DNA are known in the art and have been discussed above. Preferably, the promoter sequence is selected to provide optimal transcriptional activity of the particular RNA polymerase used.

Criteria for such selection, i.e., a particular promoter sequence particularly favored by a particular RNA polymerase, is also known in the art. For example, the sequences of the promoters for transcription by T7 DNA dependent RNA polymerase and SP6 are known in the art. The promoter sequence can be from a prokaryotic or eukaryotic source.

In some embodiments, the PTO comprises an intervening sequence between a propromoter sequence and a portion capable of hybridizing to the 3′ end of the primer extension product. Suitable length of the intervening sequence can be empirically determined, and can be at least about 1, 2, 4, 6, 8, 10, 12, 15 nucleotides. Suitable sequence identity of the intervening sequence can also be empirically determined, and the sequence is designed to preferably, but not necessarily, enhance degree of amplification as compared to omission of the sequence. In one embodiment, the intervening sequence is a sequence that is designed to provide for enhanced, or more optimal, transcription by the RNA polymerase used. Generally, the sequence is not related (i.e., it does not substantially hybridize) to the oligonucleotide template. More optimal transcription occurs when transcriptional activity of the polymerase from a promoter that is operatively linked to said sequence is greater than from a promoter that is not so linked. The sequence requirements for optimal transcription are generally known in the art as previously described for various DNA dependent RNA polymerases, such as in U.S. Pat. Nos. 5,766,849 and 5,654,142, and can also be empirically determined.

In another embodiment, the PTO comprises a sequence that is 5′ to the propromoter sequence, i.e., the PTO comprises additional nucleotides (which may or may not be transcriptional regulatory sequences) located 5′ to the propromoter sequence. Generally, but not necessarily, the sequence is not hybridizable to the primer extension product.

In one embodiment, the PTO cannot function efficiently as a primer for nucleic acid extension. Techniques for blocking the primer function of the PTO include any that prevent addition of nucleotides to the 3′ end of the PTO by a DNA polymerase. Such techniques are known in the art, including, for example, substitution or modification of the 3′ hydroxyl group, or incorporation of a modified nucleotide, such as a dideoxynucleotide, in the 3′-most position of the PTO that is not capable of anchoring addition of nucleotides by a DNA polymerase. It is also possible to block the 3′ end using a label, or a small molecule which is a member of a specific binding pair, such as biotin.

The length of the portion of the PTO that hybridizes to the displaced primer extension product is preferably from about 5 to about 50 nucleotides, more preferably from about 10 to about 40 nucleotides, even more preferably from about 15 to about 35 nucleotides, and most preferably from about 20 to 30 nucleotides. In some embodiments, the hybridizing portion is at least about any of the following: 3, 5, 10, 15, 20; and less than about any of the following: 30, 40, 50, 60. The complementarity of the hybridizing portion is preferably at least about 25%, more preferably at least about 50%, even more preferably at least about 75%, and most preferably at least about 90%, to its intended binding sequence on the displaced primer extension 5 product.

5. DNA Polymerase, Ribonuclease and RNA Polymerase

The amplification methods of the invention employ the following enzymes: a DNA polymerase, ribonuclease such as RNase H, and, optionally a DNA dependent RNA polymerase. These components are described in, e.g., U.S. Pat. No. 6,251,639.

DNA polymerases for use in the methods and compositions of the present invention are capable of effecting extension of the composite primer according to the methods of the present invention. Accordingly, a preferred polymerase is one that is capable of extending a nucleic acid primer along a nucleic acid template that is comprised at least predominantly of deoxynucleotides. The polymerase should be able to displace a nucleic acid strand from the polynucleotide to which the to-be-displaced strand is bound. Preferably, the DNA polymerase has high affinity for binding at the 3′-end of an oligonucleotide hybridized to a nucleic acid strand. Preferably, the DNA polymerase does not possess substantial nicking activity. Preferably, the polymerase has little or no 5′→3′ exonuclease activity so as to minimize degradation of primer, or primer extension polynucleotides. Generally, this exonuclease activity is dependent on factors such as pH, salt concentration, whether the template is double stranded or single stranded, and so forth, all of which are familiar to one skilled in the art. Mutant DNA polymerases in which the 5′→3′ exonuclease activity has been deleted, are known in the art and are suitable for the amplification methods described herein. Mutant DNA polymerases which lack both 5′ to 3′ nuclease and 3′ to 5′ nuclease activities have also been described, for example, exo^{-/-}Klenow DNA polymerase. Suitable DNA polymerases for use in the methods and compositions of the present invention include those disclosed in U.S. Pat. Nos. 5,648,211 and 5,744,312, which include exo⁻ Vent (New England Biolabs), exo⁻ Deep Vent (New England Biolabs), Bst (BioRad), exo⁻ Pfu (Stratagene), Bca (Panvera), sequencing grade Taq (Promega), and thermostable DNA polymerases from thermoanaerobacter thermohydrosulfuricus. The DNA polymerase displaces primer extension products from the oligonucleotide template in at least about 25%, or at least about 50%, or at least about 75%, or at least about 90%, of the incidence of contact between the polymerase and the 5′ end of the primer extension product. In some embodiments, the use of thermostable DNA polymerases with strand displacement activity is preferred. Such polymerases are known in the art, such as described in U.S. Pat. No. 5,744,312 (and references cited therein). Preferably, the DNA polymerase has little.to no proofreading activity.

The agent for cleaving RNA from a RNA/DNA hybrid may be an enzyme, for example, a ribonuclease. Preferably, the ribonuclease cleaves ribonucleotides regardless of the identity and type of nucleotides adjacent to the ribonucleotide to be cleaved. It is preferred that the ribonuclease cleaves independent of sequence identity. Examples of suitable ribonucleases for the methods and compositions of the present invention are well known in the art, including ribonuclease H (RNase H).

The DNA-dependent RNA polymerase for use in the methods and compositions of the present invention are known in the art. Either eukaryotic or prokaryotic polymerases may be used. Examples include T7, T3 and SP6 RNA polymerases. Generally, the RNA polymerase selected is capable of transcribing from the promoter sequence provided by the TSO or PTO as described herein. Generally, the RNA polymerase is a DNA dependent polymerase, which is preferably capable of transcribing from a single stranded DNA template so long as the promoter region is double stranded.

In general, the enzymes used in the methods and compositions of the present invention should not produce substantial degradation of the nucleic acid components of said methods and compositions.

Single-stranded nucleic acid or DNA binding protein (“SSB”) can be used to enhance the efficiency of the hybridization and the denaturation of the primer and the oligonucleotide template. Examples of SSBs suitable for use in the present invention include E. coli SSB (“EcoSSB”), T4 gene 32 protein, T7 SSB, Coliphage N4 SSB, calf thymus unwinding protein and adenovirus DNA binding protein. SSBs may decrease or remove secondary structure in ssDNA. EcoSSB is stable up to 100° C., and appears to be less sensitive to salt concentrations than SSB32. EcoSSB also has a lower tendency to aggregate than SSB32. Generally, EcoSSB, SSB32 and phage T7 SSB may improve hybridization of polynucleotides with complementary nucleic acid sequences. SSB32 may be useful for improving the specificity of hybridization, and can be used in embodiments where the presence or absence and/or quantity of a plurality of analytes is to be determined.

6. Reaction Conditions

Appropriate reaction media and conditions for carrying out the methods of the present invention are those that permit binding of analyte and binding partner, and nucleic acid amplification according to the methods of the present invention. Such media and conditions are known to persons of skill in the art, and are described in various publications, such as U.S. Pat. Nos. 5,679,512 and 6,251,639, and PCT Pub. No. WO99/42618.

For example, a buffer may be Tris buffer, although other buffers can also be used as long as the buffer components are non-inhibitory to enzyme components of the methods of the invention. The pH is preferably from about 5 to about 11, more preferably from about 6 to about 10, even more preferably from about 7 to about 9, more preferably from about 7.5 to about 8.5, and most preferably about 8.5. The reaction medium can also include bivalent metal ions such as Mg²⁺ or Mn²⁺, at a final concentration of free ions that is within the range of from about 0.01 to about 10 mM, and most preferably from about 1 to 5 mM. The reaction medium can also include other salts, such as KCl, that contribute to the total ionic strength of the medium. For example, the range of a salt such as KCl is preferably from about 0 to about 100 mM, more preferably from about 0 to about 75 mM, and most preferably from about 0 to about 50 mM. The reaction mixture may also contain a ssDNA binding protein; for example, it may contain 3 ug T4gp32 (USB). The reaction medium can further include additives that could affect performance of the amplification reactions, but that are not integral to the activity of the enzyme components of the methods. Such additives include proteins such as BSA, and non-ionic detergents such as NP40 or Triton. Reagents, such as DTT, that are capable of maintaining enzyme activities can also be included; for example, DTT may be included at a concentration of about 1 to about 5 mM. Such reagents are known in the art. Where appropriate, an RNase inhibitor (such as Rnasine) that does not inhibit the activity of the RNase employed in the method can also be included.

Any aspect of the methods of the present invention can occur at the same or varying temperatures. Preferably, the reactions are performed isothermally, which avoids the cumbersome thermocycling process. The amplification reaction is carried out at a temperature that permits hybridization of the oligonucleotides (primer, optionally PTO, or, optionally, TSO) of the present invention to the oligonucleotide template and that does not substantially inhibit the activity of the enzymes employed. The temperature can be in the range of preferably about 25° C. to about 85° C., more preferably about 30° C. to about 75° C., more preferably about 37° C. to about 70° C., and most preferably at about 55° C. In some embodiments that include RNA transcription, the temperature for the transcription steps is lower than the temperature(s) for the preceding steps. In these embodiments, the temperature of the transcription steps can be in the range of preferably about 25° C. to about 85° C., more preferably about 30° C. to about 75° C., and most preferably about 37° C. to about 70° C.

Nucleotide and/or nucleotide analogs, such as deoxyribonucleoside triphosphates, that can be employed for synthesis of the primer extension products in the methods of the invention are provided in the amount of from preferably about 50 to about 2500 μM, more preferably about 100 to about 2000 μM, even more preferably about 500 to about 1700 μM, and most preferably about 800 to about 1500 μM. Deoxyribose nucleoside triphosphates (dNTPs) may be used at a concentration of, for example, about 250 to about 500 uM. In some embodiments, a nucleotide or nucleotide analog whose presence in the primer extension strand enhances displacement of the strand (for example, by causing base pairing that is weaker than conventional AT, CG base pairing) is included. Such nucleotide or nucleotide analogs include deoxyinosine and other modified bases, all of which are known in the art. Nucleotides and/or analogs, such as ribonucleoside triphosphates, that can be employed for synthesis of the RNA transcripts in the methods of the invention are provided in the amount of from preferably about 0.25 to about 6 mM, more preferably about 0.5 to about 5 mM, even more preferably about 0.75 to about 4 mM, and most preferably about 1 to about 3 mM.

The oligonucleotide components of the amplification reactions of the invention are generally in excess of the number of oligonucleotide template sequence to be amplified. They can be provided at about or at least about any of the following: 10, 10², 10⁴, 10⁶, 10⁸, 10¹⁰, 10¹² times the amount of oligonucleotide template. Composite primers, TSO, PTO and the blocker sequence can each be provided at about or at least about any of the following concentrations: 50 nM, 100 riM, 500 nM, 1000 nM, 2500 nM, 5000 nM.

In one embodiment, the foregoing components, and others as needed to promote analyte binding to binding partner, as will be apparent to one of skill in the art, are added simultaneously at the initiation of the amplification process. In another embodiment, components are added in any order prior to or after appropriate timepoints during the analyte binding to binding partner, and during the amplification process, as required and/or permitted by the binding and/or amplification reaction. Such timepoints, some of which are noted below, can be readily identified by a person of skill in the art. The enzymes used for nucleic acid amplification according to the methods of the present invention can be added to the reaction mixture either prior to the analyte-binding partner binding step, simultaneous with the analyte-binding partner binding step, following the analyte-binding partner binding step, or following hybridization of the primer and/or blocker sequence to the target DNA, as determined by considerations known to the person of skill in the art.

7. Detectable Identifying Characteristics of Amplification Product

The product of the amplification step (or “amplification product”) can be either displaced primer extension product comprising polynucleotide (typically, DNA) complementary or identical to the primer extension region and part or all of the DNA portion of the composite primer (if amplification is carried out without an RNA transcription step) or sense RNA corresponding to part or all of the DNA portion of the composite primer and the primer extension region of the oligonucleotide template. In some embodiments, the amplification product, whether DNA or RNA, possesses at least one detectable identifying characteristic. Appropriate detectable identifying characteristics to be incorporated in amplification product can be determined by one skilled in the art, in view of and based on the context of the components (such as type and/or form of the dNTP(s) provided, and/or type of label associated with the dNTPs provided and the primer). It is appreciated that one or more detectable identifying characteristics may be used to characterize an amplification product, and that characterization may be performed iteratively. It is also understood that more than one amplification product, from more than one oligonucleotide template, may be present in the reaction mixture, and that each amplification product may have its own detectable identifying characteristic. In some embodiments, detection is accomplished through detection of amplification products such as, e.g., pyrophosphate, and does not require detection of an identifiable characteristic of the DNA or RNA produced by amplification.

Examples of detectable identifying characteristics for amplification products include size of the product (since the sizes of the various components which contribute to the amplification product are known, and thus the size of the amplification product is known), sequence of the amplification product (since the sequence of the amplification product is known), and detectable signal associated with the amplification product. Detectable signal may be associated with a label on a deoxyribonucleoside triphosphate or ribonucleotide triphosphate or analog thereof that is incorporated during primer extension or RNA transcription Detectable signal may also be associated with interaction of two labels. For example, one label may be on a deoxyribonucleoside triphosphate or analog thereof that is incorporated during primer extension and another label is on a deoxyribonucleoside triphosphate or analog thereof located in the primer portion of the primer extension product, or both labels may be on deoxyribonucleoside triphosphates or analogs thereof that are incorporated during primer extension. It is understood by one skilled in the art that while the preceding discussion addresses detection of accumulated amplification products comprising a detectable identifying characteristic(s), absence of accumulated amplification products comprising a detectable identifying characteristic(s) is also informative, indicating absence of analyte in the sample.

In one example, characterization of amplification products based on size may be used when determining whether a plurality of analytes, and/or a plurality of binding partner binding sites on a single analyte, are present or absent in a sample. Various combinations of length of the primer extension regions, length of DNA portion of primer, and length of 5′ region of TSO or of the PTO, if used, may be employed to produce amplification products of different sizes for each of the different oligonucleotides attached to different binding partners. Thus, there can be the creation of different sized amplification products for each analyte, and/or each binding area of an analyte, and/or each subunit of a multisubunit analyte.

In another example, detection and/or characterization of amplification product can be based on the sequence of the amplification product, which can be designed to be unique for all or part of each amplification product or groups of amplification products. Methods of detection based on sequence are well known in the art. Sequence-based detection methods include hybridization of the amplification product to specific oligonucleotides, for example, immobilized on an array. This method is particularly useful when a plurality of amplification products are produced.

In yet another example, characterization of amplification product can be based on detection of a signal, or lack thereof, from amplification product. Such a signal may be associated with incorporation of labeled dNTP(s), or analog(s) thereof into the product. For example, in the scenarios illustrated above, a labeled dNTP corresponding to an analyte could be incorporated into an amplification product. Detection of product with the signal generated by the label indicates the presence of that analyte.

The amplification products may be labeled by incorporation of labeled nucleotide during replication, may be labeled subsequent to replication by elongation with labeled nucleotides (end label using terminal transferase), labeled by incorporation of label using a labeled primer, or indirectly labeled by binding of sequence-specific labeled probe.

Labels suitable for use in the methods of this invention are known in the art, and include, for example, fluorescent dye labels and isotopic labels. Homogeneous detection of the amplification product can also be employed. For example, the optical properties of a label associated with a dNTP can be altered subsequent to incorporation of the labeled dNTP into an amplification product. Such a label includes fluorescent dyes that undergo fluorescence polarization between being attached to free dNTPs and being incorporated in a polynucleotide. See, e.g. U.S. Pat. No. 6,326,142, and references cited therein.

Another example of homogeneous detection is based on alteration of spectral properties of a label by means of energy transfer. When a primer is labeled by a donor or acceptor dye, for example, and/or the dNTPs, or their analogs, are labeled with acceptor or donor dyes, respectively, incorporation of the labeled dNTPs into the product enables energy transfer between the donor-acceptor dyes, thus resulting in specific detectable spectral properties of the attached dyes. These dyes are known in the art, as described in, for example, U.S. Pat. No. 4,996,143 (e.g., fluorescein and Texas Red donor acceptor dye pair), and U.S. Pat. No. 5,688,648. Other label combinations are also possible. For example, two ligands (such as digoxigenin and biotin) each attached to different parts of an amplification product (generally, primer and non-primer portion, or for RNA transcripts, different nucleotides in the transcript) can be brought into close proximity in the context of an amplification product. Binding of the two ligands with their corresponding antibodies which are differentially labeled can be detected due to the interaction of the labels. For instance, if the two different labels are a photosensitizer and a chemiluminescent acceptor dye, the interaction of the labels can be detected by the luminescent oxygen channeling assay as described in U.S. Pat. No. 5,340,716. Other interacting label pairs useful in the present invention are known in the art, see, e.g., U.S. Pat. Nos. 5,340,716; 3,999,345; 4,174,384; and 4,261,968 (Ullman et al.); and 5,565,322 (Heller et al.); 5,709,994 (Pease et al.); and 5,925,517 (Tyagi et al.). Examples of ligands in which one member modulates the signal of another include a fluorescent label, a chemiluminescent label, and a bioluminescent label. In some embodiments, the ligands produce little or no signal when in close proximity, and a greater signal when separated. In other embodiments, the ligands may generate a signal when in close proximity and generate less or no signal when separated.

For nucleic acids, the use of light-up probes in nucleic acid analysis allows one member of a complementary pair to be labeled in such a way that binding of the two strands results in a large increase in fluorescence signal. The use of such probes is known in the art and discussed in, e.g., U.S. Pat. No. 6,329,144; Svanvik et al., Ana. Biochem. (2000) 281:26-35.

As will be apparent to one of skill in the art, the idea of interacting labels that result in modulation of signal may be more generally applied. For example, an enzyme that catalyzes a reaction producing a detectable product may be paired with its cofactor or its inhibitor, or multiple subunits of an enzyme may be paired. It will be apparent that in the case of cofactor or multiple subunits, pairing of the entities in close proximity increases signal (e.g., a detectable product whose production is catalyzed by the enzyme), whereas in the case of inhibitor, pairing of the entities in close proximity decreases signal.

In addition, the amplification product can be detected by hybridizing it to a labeled oligonucleotide. In one embodiment, amplification product contains one member of a interacting label pair and the labeled oligonucleotide to which the amplification product hybridizes contains the other member. In another embodiment, one member of an interacting label pair is used in an attachment oligonucleotide to which amplified product binds and the other member is used in a third (capture) oligonucleotide. The third oligonucleotide may be immobilized on a solid surface and the hybridization of the labeled amplification product-attachment oligonucleotide hybrid to the third (capture) oligonucleotide results in an altered detectable signal due to the interaction of the labels on the amplification product and the third oligonucleotide. The immobilized oligonucleotide may be a part of an array of a plurality of oligonucleotides, each oligonucleotide being specific for a amplification product comprising a distinct sequence.

Detection of these detectable identifying characteristics can be achieved by a variety of methods evident to one skilled in the art. Methods of determining size and sequencing of polynucleotides (such as an attached oligonucleotide combination product) are known in the art. Methods of detecting detectable signals are known in the art, and are described above.

In one embodiment, the resultant amplification products in a reaction mixture are separated for analysis on a suitable matrix. Any of a number of methods can be used to effect the separation, as described in, for example, McIntosh et al., infra. Such methods include, but are not limited to, oligonucleotide array hybridization, mass spectrometry, flow cytometry, HPLC, FPLC, size exclusion chromatography, affinity chromatography, and gel electrophoresis.

It is appreciated that while the preceding discussion describes detection of amplification product comprising a detectable identifying characteristic(s), the absence of accumulation of amplification product comprising a detectable identifying characteristic(s) is also informative, indicating absence of analyte in the sample.

Reaction conditions, components, and other experimental parameters as well as illustrative embodiments in this section are generally as described herein.

B. Methods of Amplification of the Oligonucleotide Template

In one aspect of the amplification step of the methods of the present invention, a method for amplifying a nucleotide sequence complementary to a portion of the oligonucleotide template is provided. In this method, isothermal linear amplification is achieved. In another aspect, the amplified portion of the oligonucleotide template is itself amplified. In another aspect, a method for amplifying a portion of the oligonucleotide template wherein the amplified product is sense RNA is provided. The latter two aspects are sometimes referred to herein as an “enhanced” linear amplification methods.

The methods for amplifying the primer extension template region generally comprise using an RNA/DNA composite primer, optionally a termination sequence, and, in embodiments in which transcription is used (i.e., enhanced linear amplification), a propromoter oligonucleotide sequence.

The methods work as follows (see, e.g., U.S. Pat. No. 6,251,639): a composite RNA/DNA primer forms the basis for replication of the primer extension template region of the oligonucleotide template. In some optional embodiments, a termination sequence provides the basis for an endpoint for the replication by either diverting or blocking further replication along the template oligonucleotide strand. As described below, in some optional embodiments, the polynucleotide comprising a termination sequence is a template switch oligonucleotide (TSO), which contains sequences that are not of sufficient complementarity to hybridize to the template oligonucleotide strand (in addition to sequences which are of sufficient complementary to hybridize); in other optional embodiments, the termination sequence comprises primarily sequences that are of sufficient complementarity to hybridize to the template oligonucleotide strand. DNA polymerase effects copying of the primer extension region from the primer. An enzyme which cleaves RNA from an RNA/DNA hybrid (such as RNaseH) cleaves (removes) RNA sequence from the hybrid, leaving sequence on the template oligonucleotide strand available for binding by another composite primer. Another strand is produced by DNA polymerase, which displaces the previously replicated strand, resulting in displaced extension product.

Accordingly, the linear amplification step of the methods of the invention generally comprises combining and reacting the following: (a) a single-stranded oligonucleotide template (attached to the binding partner) comprising a primer extension template region; (b) a composite primer comprising an RNA portion and a 3′ DNA portion; (c) a DNA polymerase; (d) deoxyribonucleoside triphosphates or suitable analogs; (e) an enzyme, such as RNaseH, which cleaves RNA from an RNA/DNA duplex; and (f) optionally, a polynucleotide comprising a termination sequence, such as any of those described herein, which comprises a portion (or region) which hybridizes to the oligonucleotide template. A termination sequence is used if transcription-based amplification (i.e., enhanced linear amplification, see below) is also used. The combination is subjected to suitable conditions such that (a) the composite primer (and, optionally, a polynucleotide comprising a termination sequence) hybridizes to the oligonucleotide template; (b) primer extension occurs from the composite primer, to form a duplex; (c) RNaseH cleaves RNA of the composite primer from the RNA/DNA duplex; (d) another composite primer hybridizes to the oligonucleotide template, and another round of primer extension (mediated by DNA polymerase) occurs, displacing the strand already copied from the oligonucleotide template.

Methods of enhanced linear amplification further include either steps for a second round of composite primer-based amplification or a round of transcription-based amplification.

The composite primer-based enhanced amplification further includes the following steps: a second composite primer binds to the displaced primer extension product. The primer serves as an initiation point for DNA-polymerase-catalyzed polymerization along the displaced primer extension product. The second composite primer is cleaved by an RNA cleaving agent. The polymerization product is displaced by another round of polymerization initiated by binding of another second composite primer.

In transcription-based enhanced linear amplification, the following steps are included in addition to the linear amplification: a polynucleotide comprising a propromoter and a region which hybridizes to the displaced primer extension product (which can be, for example, a propromoter template oligonucleotide or a template switch oligonucleotide), which contains sequences of sufficient complementarity to hybridize to the 3′ end of the displaced primer extension product, binds to the displaced primer extension product. The promoter drives transcription (via DNA-dependent RNA polymerase) to produce sense RNA products.

Thus, if transcription-based enhanced linear amplification is used, the following is also included in the amplification reaction (either at the same time as those components listed above or added separately): (e) a polynucleotide comprising a propromoter sequence (which can be in any of a number of forms, as described herein) and a region which hybridizes to the displaced primer extension product; (f) ribonucleoside triphosphates or suitable analogs; and (g) RNA polymerase, under conditions such that transcription of the displaced strand can occur. Details regarding the various components of the methods of the present invention are provided below.

The following are examples of the amplification methods of the invention. It is understood that various other embodiments may be practiced, given the general description provided above. For example, reference to using a composite primer means that any of the composite primers described herein may be used. In one embodiment, a method of enhanced isothermal linear nucleic acid sequence amplification which is TSO-based is provided (hereinafter “Method 1”). In another embodiment, a method of enhanced isothermal linear nucleic acid sequence amplification which is blocker sequence and PTO-based is provided (hereinafter “Method 2”).

1. Linear Nucleic Acid Sequence Amplification Resulting in DNA Amplification Product

When not linked to transcription, the amplification method of the invention provides for isothermal linear amplification of a portion of the oligonucleotide template. The method utilizes a single composite primer. In one embodiment, the method also employs a termination sequence, such as a blocker sequence as described in Method 2, or a TSO, as described in Method 1. Methods 1 and 2 are described below. Insofar as the linear amplification is not linked to transcription, the components and steps leading to formation of a complex comprising a promoter sequence for a DNA dependent RNA polymerase, are not included.

The termination sequence (either TSO or blocker sequence component, if used) is added for producing a product of defined 3′-end. In some embodiments, sequence(s) within the oligonucleotide template 5′ of the primer binding site inhibits nucleic acid polymerization such that termination of primer extension is achieved. Such sequences are known in the art, for example, GC rich sequences, or can be empirically determined.

When this feature is not desired, the isothermal linear amplification according to the methods of the invention can be carried out without a termination sequence.

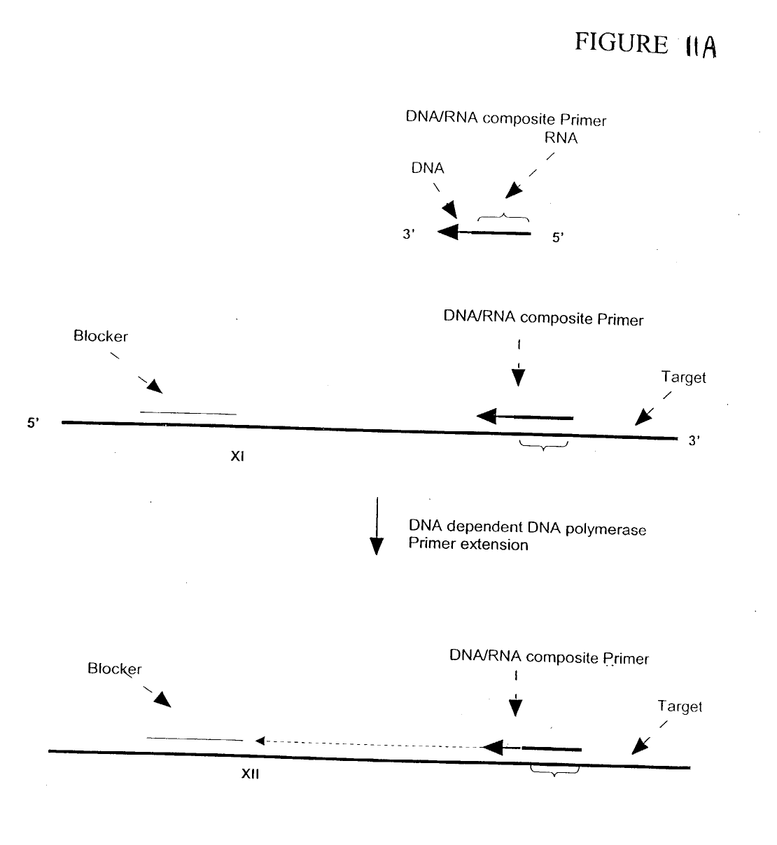

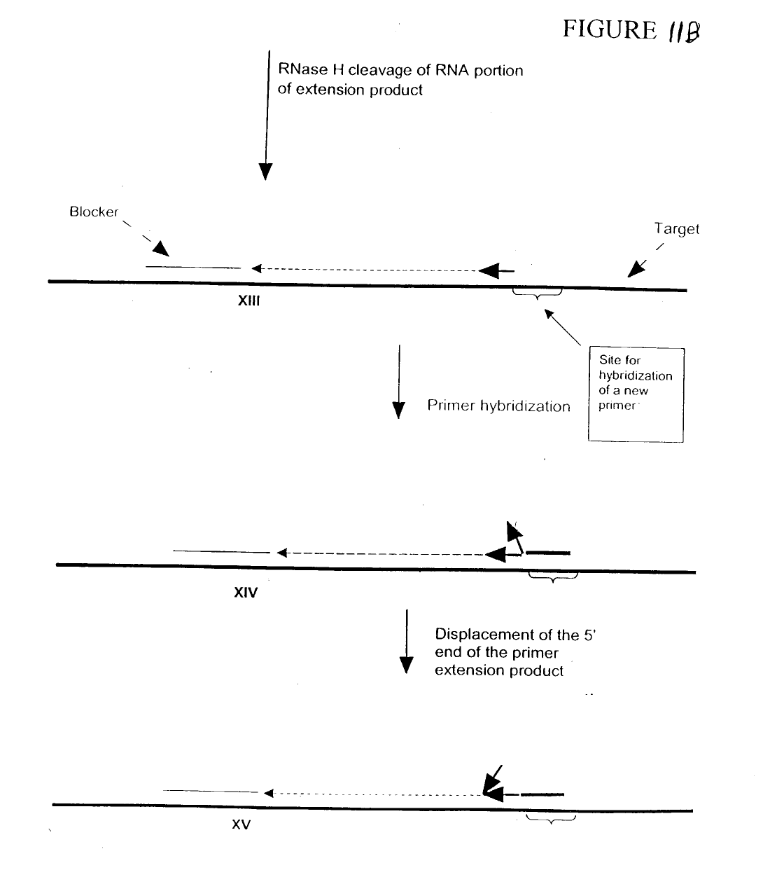

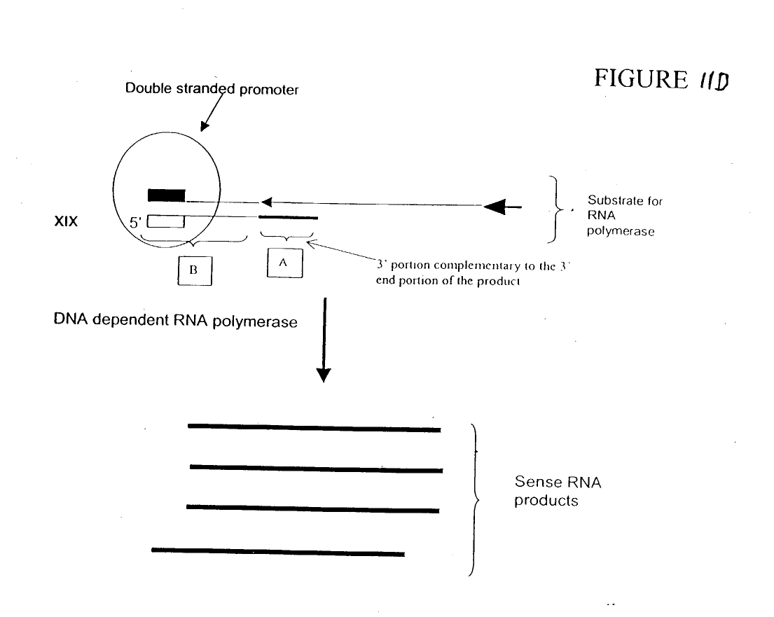

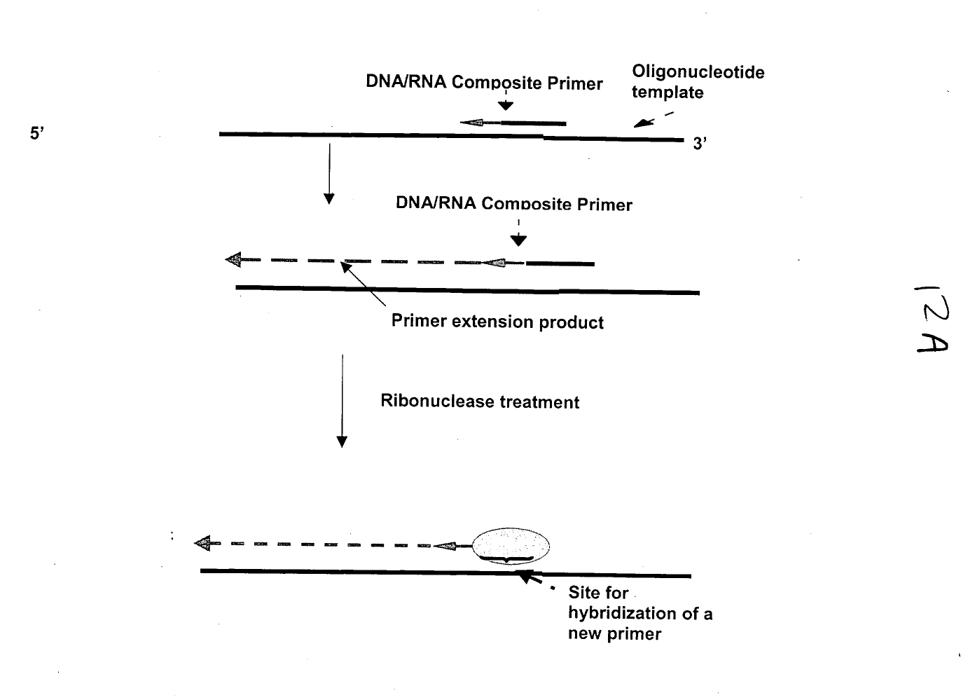

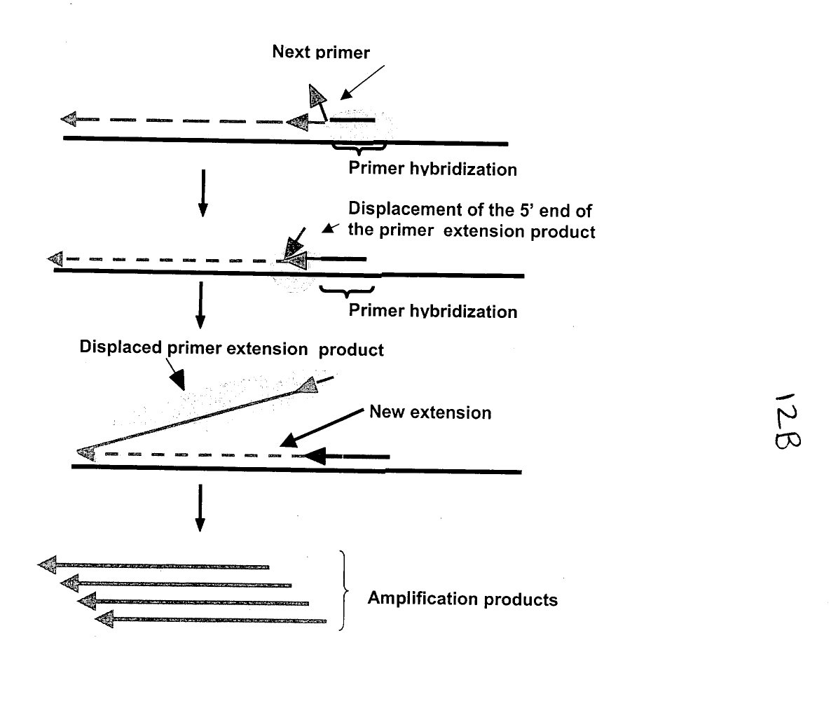

The isothermal linear amplification further utilizes two enzymes, a DNA polymerase and a ribonuclease such as RNase H. Schematic description of the linear isothermal nucleic acid amplification of the invention is shown in FIGS. 10A-C and in FIGS. 12A and 12B. FIGS. 10A-C show the amplification with a blocker sequence present while FIGS. 12A and 12B show amplification without a blocker sequence.

Similar to Methods 1 and 2 as described below, the linear amplification method is designed to amplify a single stranded DNA oligonucleotide template.

As shown in FIGS. 10A-C and 12A and 12B, the linear isothermal amplification method of the invention comprises steps similar to the initial steps of the enhanced linear amplification methods (Methods 1 and 2) described below and in FIGS. 9A-C and 11A-D. The oligonucleotide template is combined with a composite primer, DNA polymerase, a ribonuclease such as RNase H (FIG. 12A) and optionally a blocker sequence component or TSO (FIG. 10A), as described above. In one embodiment, each amplification reaction includes a mixture of composite primers, wherein the primers represent two or more non-identical sequences that are of low or no homology, and wherein the primers preferentially hybridize to different oligonucleotide template sequences or different sites along the same oligonucleotide template strand. Advantages of this embodiment include multiplex detection and/or analysis of a plurality of analytes through amplification of a plurality of oligonucleotide template species in a single amplification reaction.

FIGS. 10A-C illustrates an embodiment that includes a termination sequence and FIGS. 12A and 12B illustrate an embodiment without blocker. For simplicity, only the termination sequence (TSO or blocker sequence) embodiment will be described (FIGS. 10A-C). The embodiment without termination sequence is the same except for the absence of termination sequence, which is not necessary when polymerization is terminated by steric considerations or by reaching the end of the oligonucleotide template (FIGS. 12A and 12B). The composite primer and the termination sequence (TSO or blocker sequence component) hybridize to the same oligonucleotide template, to form a tri molecular complex, XX (FIG. 10A). The 3′-end of the composite primer is extended along the oligonucleotide template by the polymerase, optionally up to the site of hybridizing of the TSO or blocker sequence component, to yield complex XXI (FIGS. 10A-C). A ribonuclease such as RNase H cleaves the RNA, generally the 5′-RNA, portion of the extended primer of complex XXI (FIGS. 10A-C) to produce complex XXII (FIGS. 10A-C). A second composite primer binds to complex XXII (FIGS. 10A-C) by hybridization of the RNA, generally the 5′ RNA, portion to yield complex XXIII (FIGS. 10A-C). The free 3′ portion of the bound composite primer then displaces the 5′ end of the primer extension product and hybridizes to the oligonucleotide template to form complex XXIV (FIGS. 10A-C). The hybridization of the 3′ end of the composite primer to the oligonucleotide template is generally favored over the hybridization of the 5′ end of the primer extension product since the hybridized 3′ end of the primer is a site of binding of the DNA polymerase which will then extend the 3′ end of the primer along the oligonucleotide template. Primer extension results in displacement of the first primer extension product to yield complex XXV (FIGS. 10A-C). The process is repeated to yield multiple single stranded DNA displacement products which are generally complementary to the primer extension template region of the oligonucleotide template.

As is evident from the description of the amplifications methods, displaced primer extension products can serve as templates for further amplification. A composite primer can be hybridized to displaced primer extension products (serving as DNA templates), and extended and displaced as described herein. Accordingly, in some embodiments, the invention provides methods for amplifying a portion of an oligonucleotide template comprising: (a) hybridizing a first composite primer to a single stranded DNA template comprising the complementary sequence of a portion of the oligonucleotide template, said composite primer comprising an RNA portion and a 3′ DNA portion, wherein said single stranded DNA template is generated by a method comprising: (i) hybridizing a portion of the oligonucleotide template with a second composite primer, said second composite primer comprising an RNA portion and a 3′ DNA portion; (ii) optionally hybridizing a polynucleotide comprising a termination polynucleotide sequence to a region of the oligonucleotide template that is 5′ with respect to hybridization of the second composite primer to said oligonucleotide template; (iii) extending the second composite primer with DNA polymerase; and (iv) cleaving RNA of the annealed second composite primer with an enzyme that cleaves RNA from an RNA/DNA hybrid such that another composite primer hybridizes to the oligonucleotide template and repeats primer extension by strand displacement, whereby multiple copies of a single stranded DNA template comprising the complementary sequence of a portion of the oligonucleotide template are generated; (b) optionally hybridizing a polynucleotide comprising a termination polynucleotide sequence to a region of the template which is 5′ with respect to hybridization of the first composite primer to the template; (c) extending the first composite primer with DNA polymerase; (d) cleaving RNA of the annealed first composite primer with an enzyme that cleaves RNA from an RNA/DNA hybrid such that another composite primer hybridizes to the template and repeats primer extension by strand displacement; whereby multiple copies of a portion of the oligonucleotide template are produced.

The single stranded DNA (i.e., the displaced primer extension products) of the isothermal linear amplification method are readily detectable by any of many detection methods known in the art. Various homogeneous or heterogeneous detection methods suitable for the detection of single stranded nucleic acid molecules are described herein, including identification by size and/or migration properties in gel electrophoresis, or by hybridization to sequence-specific probes.

The detection of the amplification product is indicative of the presence of the analyte. Quantitative analysis is also feasible. For example, by comparing the amount of product amplified from a test sample containing an unknown amount of an analyte to the product of amplification of a reference sample that has a known quantity of the analyte, or of binding partner for the analyte, the amount of analyte in the test sample can be determined.

The production of at least 1, at least 10, at least about 100, at least about 1000, at least about 10⁵, at least about 10⁷, at least about 10⁹, at least about 10¹², complementary or identical copies of each copy of oligonucleotide template can be expected, thus leading to at least 1, at least 10, at least 100, at least 1000, at least about 10⁵, at least about 10⁷, at least about 10⁹, at least about 10¹²-fold enhancement with respect to each copy oligonucleotide template.

2. Enhanced Linear Amplification Based on Further DNA Replication from the Displaced Primer Extension Product.

As is evident from the description of the amplifications methods, displaced primer extension products can serve as templates for further amplification. Another composite primer can be hybridized to displaced primer extension products (serving as DNA templates), and extended and displaced as described herein. Accordingly, in some embodiments, the invention provides methods for amplifying a portion of an oligonucleotide template comprising: (a) hybridizing a first composite primer to a single stranded DNA template comprising the complementary sequence of a portion of the oligonucleotide template, said composite primer comprising an RNA portion and a 3′ DNA portion, wherein said single stranded DNA template is generated by a method comprising: (i) hybridizing a portion of the oligonucleotide template with a second composite primer, said second composite primer comprising an RNA portion and a 3′ DNA portion; (ii) extending the second composite primer with DNA polymerase; and (iv) cleaving RNA of the annealed second composite primer with an enzyme that cleaves RNA from an RNA/DNA hybrid such that another composite primer hybridizes to the oligonucleotide template and repeats primer extension by strand displacement, thus displacing the primer extension product, whereby multiple copies of a single stranded DNA template comprising the complementary sequence of a portion of the oligonucleotide template are generated; (b) extending the first composite primer with DNA polymerase; (c) cleaving RNA of the annealed first composite primer with an enzyme that cleaves RNA from an RNA/DNA hybrid such that another composite primer hybridizes to the oligonucleotide template and repeats primer extension by strand displacement, thus displacing the primer extension product; whereby multiple copies of a portion of the oligonucleotide template are produced.

The single stranded DNA (i.e., the displaced primer extension products) of the isothermal linear amplification method are readily detectable by any of many detection methods known in the art, as described above.

The detection of the amplification product is indicative of the presence of the analyte. Quantitative analysis is also feasible. For example, by comparing the amount of product amplified from a test sample containing an unknown amount of an analyte to the product of amplification of a reference sample that has a known quantity of the analyte, or a known quantity of binding partner for the analyte that is attached to the oligonucleotide template to be amplified, the amount of analyte in the test sample can be determined.

The production of at least 1, at least 10, at least about 100, at least about 1000, at least about 10⁵, at least about 10⁷, at least about 10⁹, at least about 10¹², complementary or identical copies of each copy of oligonucleotide template can be expected, thus leading to at least 1, at least 10, at least 100, at least 1000, at least about 10⁵, at least about 10⁷, at least about 10⁹, at least about 10¹²-fold enhancement with respect to each copy oligonucleotide template.

3. Enhanced Linear Amplification Based on Transcription and Resulting in Sense RNA Amplification Product

The present invention also provides methods for amplifying a portion of an oligonucleotide template wherein the amplified product is RNA containing the sense sequence (i.e., same sequence as the portion of the oligonucleotide template). Amplification of a portion of the oligonucleotide template according to Method 1, which results in the generation of a unique intermediate amplification product comprising oligonucleotide template and template switch oligonucleotide (TSO)-related portions, provides for coupling of the linear amplification to transcription.

The complex formed by the hybridization of the template switch oligonucleotide and the displaced primer extension product is a substrate for transcription by the RNA polymerase, which generates an RNA product of the same sense as the initial portion of the oligonucleotide template. Similarly, amplification of a portion of the oligonucleotide template according to Method 2 results in formation of a displaced primer extension product which when hybridized to the promoter template oligonucleotide forms a complex, which is a substrate for the RNA polymerase. As in Method 1, this process results in coupling of the linear amplification to transcription. The production of preferably at least about 1, more preferably at least about 50, even more preferably at least about 75, still more preferably at least about 100, and most preferably at least about 1000, RNA transcript products from each primer extension product is expected, thus leading to preferably at least about 1, more preferably at least about 50, even more preferably at least about 75, still more preferably at least about 100, and most preferably at least about 1000-fold enhancement with respect to the non-transcription linked methods of amplification.

Below are two exemplary methods.

a. Method 1—TSO-Based Enhanced Linear Nucleic Acid Amplification

In one embodiment, the TSO-based linear amplification method of the present invention is linked to transcription from the primer extension products to provide enhanced nucleic acid amplification. A schematic description of this novel amplification method, Method 1, is shown in FIGS. 9A-C.

The TSO-based nucleic acid amplification method of the invention employs a single composite primer, as described above. A second oligonucleotide optionally used in the amplification method of the invention is a template switch oligonucleotide (TSO), also as described above. In some embodiments, a TSO is not required and instead the promoter sequence is provided by an oligonucleotide that binds to displaced template amplification product. The amplification method of the invention employs the following enzymes: a DNA polymerase, a ribonuclease such as RNase H, and a DNA dependent RNA polymerase.

The new TSO-based enhanced linear amplification method of the present invention can produce multiple copies of an RNA product that are sense to a portion of the oligonucleotide template sequence.

The oligonucleotide template is combined with the composite primer, a TSO oligonucleotide, DNA polymerase, ribonuclease such as RNase H, a DNA dependent RNA polymerase, and nucleotides, such as deoxyribonucleoside triphosphates (dNTPs) and ribonucleoside triphosphates (rNTPs), in a reaction medium suitable for nucleic acid hybridization and amplification, as known in the art. Suitable reaction medium and conditions are as described above. In one embodiment, transcription is performed at a different temperature, generally lower, than that of the preceding steps. In another embodiment, all the steps of the methods are performed isothermally.

In one embodiment, the amplification reaction mixture includes composite primers of identical sequence. In another embodiment, each amplification reaction includes a mixture of composite primers, wherein the primers represent two or more non-identical sequences that are of low or no homology, and wherein the primers preferentially hybridize to different oligonucleotide templates or different sites along the same oligonucleotide template. Advantages of this embodiment include multiplex detection and/or analysis of a plurality of analytes through amplification of a plurality of oligonucleotide template species in a single amplification reaction.

In one embodiment, the TSO functions as a termination sequence and provides a propromoter sequence. In another embodiment, the TSO does not comprise a propromoter sequence. In this embodiment, a propromoter sequence is provided separately by another oligonucleotide, such as a PTO, that comprises a propromoter sequence and is hybridizable (e.g., hybridizes) to the 3′ portion of the primer extension product such that transcription of the primer extension product can occur.

The single composite primer and the TSO then hybridize to the same strand of the oligonucleotide template to be amplified. Hybridization of the two oligonucleotides to the oligonucleotide template results in the formation of the tri molecular complex I (FIGS. 9A-C).

A DNA polymerase carries out primer extension. The primer is extended along the oligonucleotide template of complex I (FIGS. 9A-C), up to the site of TSO hybridization. Template switching from the oligonucleotide template strand to the 5′ unhybridized portion of the TSO, and further primer extension along the TSO template results in the formation of the tri molecular complex II. The last comprises an oligonucleotide template, the TSO and the first primer extension product. The first primer extension product is a unique DNA comprising both an oligonucleotide template dependent portion (i.e., sequence complementary to a portion of the oligonucleotide template) and a TSO dependent portion (i.e., sequence complementary to the unhybridized portion of the TSO).

Complex II (FIGS. 9A-C) is a substrate for both an RNA polymerase and a ribonuclease such as RNase H. The DNA dependent RNA polymerase binds to the functional ds promoter of complex II and transcribes the first primer extension product to produce a sense RNA product III (FIGS. 9A-C). A ribonuclease, such as RNase H, which is specific for degradation of the RNA strand of an RNA/DNA heteroduplex, degrades the 5′ portion of the primer extension product in complex II to form the tri molecular complex IV.

Free composite primer hybridizes to the primer complementary site of the oligonucleotide template in complex IV (FIGS. 9A-C). This hybridization results in formation of complex V (FIGS. 9A-C) in which only the RNA portion, generally the 5′ RNA portion, of the primer is hybridized to the oligonucleotide template. Displacement of the 5′ most portion of the primer extension product by the 3′ DNA portion of the partially hybridized primer will result in formation of complex VI (FIGS. 9A-C), which is a substrate for a DNA polymerase. Extension of the primer along the oligonucleotide template (VII; FIGS. 9A-C) results in displacement of the first primer extension product from the complex. Repeated primer extensions and strand displacements result in generation of multiple copies of polynucleotides that are at least substantially complementary to a portion of the oligonucleotide template.

The primer extension product generated as described above is used as a template for transcription in the embodiment wherein TSO that comprises a propromoter sequence is provided. The displaced primer extension product (VIII; FIGS. 9A-C) hybridizes to free TSO oligonucleotide to form the partial duplex IX (FIGS. 9A-C). Complex (duplex) IX comprises a double stranded portion at one end and two non-complementary single strands respectively derived from the primer extension product and the TSO. The double stranded portion of this partial duplex contains a fully functional double stranded promoter for the DNA dependent RNA polymerase. The last binds to the promoter of the partial duplex IX and transcribes the primer extension product to form multiple copies of a sense RNA product X (FIGS. 9A-C).

The products of the amplification described above can be detected by either homogenous or heterogeneous detection methods, including identification by size and/or migration properties in gel electrophoresis, or by hybridization to sequence-specific probes. The detection of the amplification product is indicative of the presence of the analyte. Quantitative analysis is also feasible. For example, by comparing the amount of product amplified from a test sample containing an unknown amount of an analyte to the product of amplification of a reference sample that has a known quantity of the analyte or its binding partner, the amount of analyte in the test sample can be determined.

b. Method 2—Blocker Sequence-Based Enhanced Nucleic Acid Amplification

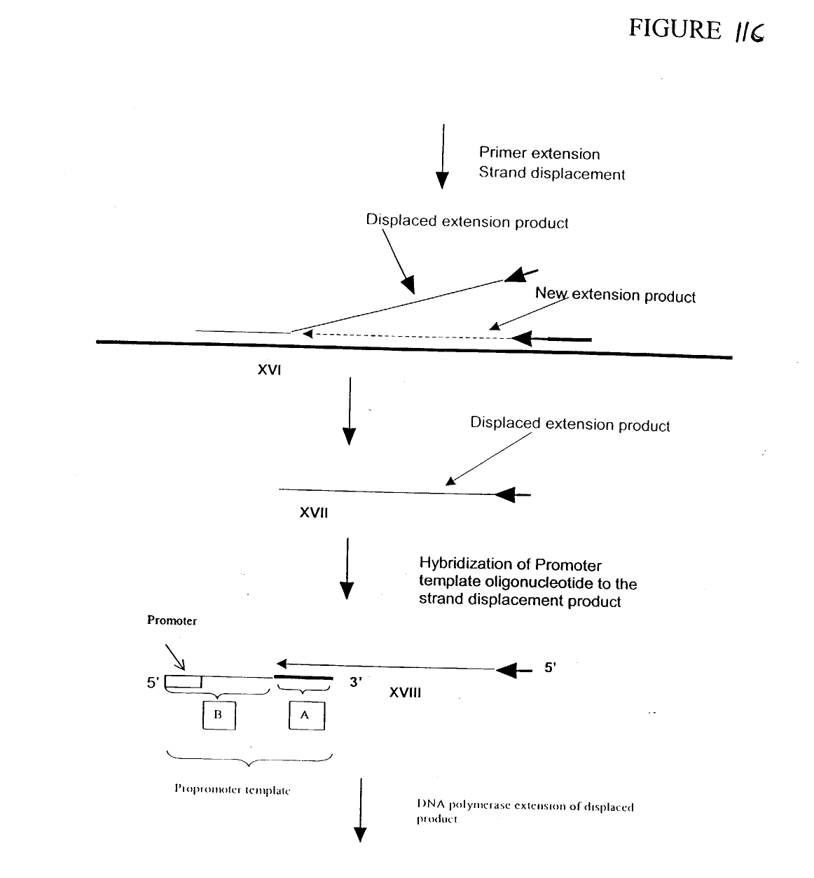

In another embodiment, the blocker sequence-based linear amplification method of the present invention is linked to transcription from the primer extension products to provide enhanced nucleic acid amplification. This alternative enhanced linear amplification, Method 2, which does not involve a template switch step, is shown in FIGS. 11A-D.

Method 2 utilizes the single composite primer, as in Method 1, as described above, an optional blocker sequence component which is either an oligonucleotide or an oligonucleotide analog, which, as described above, is further able to hybridize to a sequence on the same oligonucleotide template as the single primer, and a third oligonucleotide, the promoter template (PTO), which, as described above, comprises a 3′-portion which is able to hybridize (and is preferably complementary) to the 3′-end of the displaced extension product and a 5′-portion which includes at its 5′ end a sequence of a promoter for a DNA dependent RNA polymerase. As in the TSO described above, the sequence immediately adjacent to the promoter sequence is designed to provide for preferably optimal transcriptional activity by the RNA polymerase used in the amplification according to the method of the invention. The optional blocker sequence component is designed to hybridize to the oligonucleotide template at a site which is located upstream, towards the 5′ end of the oligonucleotide template, relative to the site of hybridization of the single primer. Stated alternatively, and as described above, the blocker sequence hybridizes to a segment of oligonucleotide template 5′ of the position in the oligonucleotide template that is complementary to the 3′ end of the primer extension product. The blocker sequence binds with sufficiently high affinity so as to block primer extension at the site of blocker hybridization to the oligonucleotide template. This optional feature provides a strong stop for primer extension by the polymerase and defines the 3′-end of the primer extension product; alternatively, and more commonly, primer extension is terminated by steric factors or by runoff from the end of the oligonucleotide template, in which cases a blocker sequence is not required.

The oligonucleotide template is combined with the single composite primer, the blocker component, the propromoter template (PTO), DNA polymerase, ribonuclease such as RNase H, a DNA dependent RNA polymerase, and nucleotides, such as NTPs (e.g., dNTPs and rNTPs), as was described for Method 1. Suitable reaction medium and conditions are as described above. In one embodiment, the transcription is performed at a different temperature, generally lower, than that of the preceding steps. In another embodiment, all the steps of the methods are performed isothermally.

In one embodiment, each amplification reaction includes composite primers of one identical sequence. In another embodiment, each amplification reaction includes a mixture of composite primers, wherein the primers represent two or more non-identical sequences that are of low or no homology, and wherein the primers preferentially hybridize to different oligonucleotide templates or different sites along the same oligonucleotide template. Advantages of this embodiment include analysis of a plurality of analytes through amplification of a plurality of oligonucleotide templates in a single amplification reaction.

The single composite primer and the blocker sequence component hybridize to the same oligonucleotide template to form a tri molecular complex. The primer is extended along the oligonucleotide template up to the site of hybridization of the blocker sequence, to form complex XII (FIGS. 11A-D).

As in Method 1, a ribonuclease, such as RNase H, cleaves RNA, generally the 5′ RNA portion, of the single composite primer of complex XII to form complex XIII (FIGS. 11A-D). As described above, the enzyme is specific for cleaving the RNA strand of an RNA/DNA hybrid, and does not digest single stranded RNA. Thus, the ribonuclease does not degrade the free composite primer. The following steps, as illustrated in FIGS. 11A-D, of primer hybridization (XIV), displacement of the 5′ end of the primer extension product by the 3′-DNA portion of the composite primer (XV), primer extension and displacement of the first primer extension product (XVI), proceed as in Method 1, to yield multiple copies of the displaced primer extension product (XVII). Unlike the displacement product of Method 1, XVII is fully complementary to a portion of the oligonucleotide template and does not comprise a 3′ end portion which is not complementary to part of oligonucleotide template. Repeated primer extensions and strand displacements result in generation of multiple copies of polynucleotides that are complementary to a portion of the oligonucleotide template.

The promoter template oligonucleotide (PTO) binds to the displaced extension product to form complex XVIII (FIGS. 11A-D), by hybridization of the 3′ end portion (A) of the propromoter template to the 3′ end of the displaced primer extension product. As described above, the 3′ end of the PTO may be blocked or not.

When the 3′ end of the propromoter template is not blocked, the template will be extended along the displaced primer extension product. The 3′ end of the displaced product will be extended by the nucleotide (DNA) polymerase along the B portion (see FIGS. 11A-D) of the hybridized propromoter template to form complex XIX, which comprises at its one end a ds promoter sequence that can be utilized by the DNA dependent RNA polymerase. Complex XIX is depicted in FIGS. 11A-D as the product of hybridization of a promoter template in which the 3′ end is blocked for extension by the polymerase. Alternatively, when the 3′ end of the promoter template is not blocked extension of the 3′ end along the displaced primer extension product results in formation of a fully double stranded complex. DNA-dependent RNA polymerase will transcribe the extended displaced primer extension product of complex XIX, in both forms (the choice of RNA polymerase must take into account its capability to transcribe from a ds and/or ss DNA template), that is to say either the partial duplex or the fully double stranded duplex forms of the complex. Multiple copies of a single stranded RNA products are produced by this transcription step.

The products of the amplification described above can be detected by either homogenous or heterogeneous detection methods, including identification by size and/or migration properties in gel electrophoresis, or by hybridization to sequence-specific probes. The detection of the amplification product is indicative of the presence of the analyte. Quantitative analysis is also feasible. For example, by comparing the amount of product amplified from a test sample containing an unknown amount of an analyte to the product of amplification of a reference sample that has a known quantity of analyte or its binding partner, the amount of analyte in the test sample can be determined.

The amplification products of the methods of the present invention can be further modified, such as through cleavage into fragments or by attachment of detectable labels, as required and/or permitted by the techniques used.

IV. Detection and/or Quantification of Amplified Products

The amplification methods described above produce amplification products. There are various ways to measure amplification product formation (if any). Some ways are direct with respect to the product. Other ways are indirect. The most universal product of amplification, whose presence may provide a simple yes-no answer as to whether amplification has occurred, and thus as to whether analyte that has bound to a binding partner is present, is pyrophosphate, which is released during DNA polymerization, and whose levels can be measured by means known in the art. Other amplification products are more specific to the amplification method used. Linear amplification produces single stranded amplification product (usually ssDNA) that is complementary to at least a portion of the oligonucleotide template. Enhanced linear amplification using further DNA replication produces, in addition to ssDNA complementary to oligonucleotide template, also ssDNA that is identical to a portion of the oligonucleotide template. Enhanced linear amplification using RNA transcription produces, in addition to ssDNA complementary to oligonucleotide template, RNA that is sense to the original portion of the oligonucleotide template. All of these products are referred to as “amplification product(s),” or “amplified product(s)” The amplification products comprise at least one detectable identifying characteristic. Examples of detectable identifying characteristics of amplified products that may serve as the basis for detection include size, sequence, and labels. The amplification products are detected by any means available to those of skill in the art. Examples of such means are given below.

A. Detection Methods

Determination of the formation of pyrophosphate may be accomplished by standard methods in the art. See e.g. Ronaghi, M. et al., 1998 Science 281: 363-365; Nyren, P., et al., 1987 Anal. Bioch. 167:235-238.

The detectable identifying characteristics of DNA and RNA products of amplification described above can be detected by methods known in the art.

Size of a polynucleotide (amplification product) can be determined by, for example, gel electrophoresis sizing and mass spectrometry (see, for example, Monforte et al., U.S. Pat. Nos. 5,830,655 and 5,700,642).

Methods of sequencing a polynucleotide (amplification product) are well-known. Suitable sequencing methods are known in the art, and include, for example, using nucleotide triphosphates that upon incorporation into amplification product terminates nucleotide polymerization. Suitable sequencing methods also include sequencing by synthesis, which is a method known in the art, wherein nucleotide sequence is determined based on whether there is extension (polymerization) of a primer hybridized to an amplified product by a polymerase when a known dNTP type is provided in the synthesis (polymerization) reaction, wherein polymerization is indicated by the formation of pyrophosphate.

Detection of sequence in an amplification product can also be achieved by methods such as limited primer extension, which are known in the art and described in, for example, U.S. Pat. Nos. 5,888,819; 6,004,744; 5,882,867; 5,710,028; 6,027,889; 6,004,745; 5,763,178; 5,011,769; 5,185,243; 4,876,187; 5,882,867; WO US88/02746; WO 99/55912; WO92/15712; WO 00/09745; WO 97/32040; WO 00/56925, and in co-pending U.S. application Ser. No. 60/255,638, filed Dec. 13, 2000.

The amplified polynucleotide products, either DNA or RNA (i.e., products of any of the amplification methods described herein), can be also be analyzed using, for example, probe hybridization techniques known in the art, such as Southern and Northern blotting. In addition, the single stranded DNA and RNA products may serve as starting material for other starting material for other analytical and/or quantification methods known in the art, such as real time PCR, quantitative TaqMan, quantitative PCR using molecular beacons, methods described in Kurn, U.S. Pat. No. 6,251,639, and the like. Thus, the invention includes those further analytical and/or quantification methods as applied to any of the products of the methods herein.

The labeled amplified products are particularly suitable for analysis (for example, detection and/or quantification) by contacting them with, for example, microarrays (of any suitable surface, which includes glass, chips, plastic), beads, or particles, that comprise suitable probes such as cDNA and/or oligonucleotide probes. Thus, the invention provides methods to characterize (for example, detect and/or quantify) an analyte by generating labeled polynucleotide (generally, DNA or RNA) products using amplification methods of the invention, and analyzing the labeled products. Analysis of labeled products can be performed by, for example, hybridization of the labeled amplification products to, for example, probes immobilized at, for example, specific locations on a solid or semi-solid substrate, probes immobilized on defined particles, or probes immobilized on blots (such as a membrane), for example arrays. Other methods of analyzing labeled products are known in the art, such as, for example, by contacting them with a solution comprising probes, followed by extraction of complexes comprising the labeled amplification products and probes from solution. The identity of the probes provides characterization of the sequence identity of the amplified products, and thus by extrapolation the identity of the analyte present in a sample. Hybridization of the labeled products is detectable, and the amount of specific labels that are detected is proportional to the amount of the labeled amplification products of a specific analyte. This measurement is useful for, for example, measuring the relative amounts of various analytes in a sample. The amount of labeled products (as indicated by, for example, detectable signal associated with the label) hybridized at defined locations on an array can be indicative of the detection and/or quantification of the corresponding analyte in the sample.

It will be appreciated that the analysis of the amplification product(s) produced is especially adaptable to hybridization by, for example oligonucleotides or polynucleotides immobilized on a solid or semi-solid surface, such as an array. Suitable nucleic acid probes will be evident to one skilled in the art, and include, for example, probes that comprise DNA, RNA, DNA and RNA, peptide nucleic acid (PNA), or any combination of DNA, RNA and/or PNA. These probes can be provided in any suitable form, including, for example, as microarrays. Methods of specific hybridization of a polynucleotide (amplification product) to polynucleotides immobilized on an array are well known in the art. As is known in the art, a microarray refers to an assembly of distinct polynucleotides or oligonucleotides immobilized at defined positions on a substrate (surface). Arrays are formed on substrates fabricated with materials such as paper, glass, plastic (e.g., polypropylene, nylon), polyacrylamide, nitrocellulose, metal, silicon, optical fiber, polystyrene, or any other suitable solid or semi-solid support, and configured in a planar (e.g., glass plates, silicon chips) or three-dimensional (e.g., pins, fibers, beads, particles, microtiter wells, capillaries) configuration.

Polynucleotides or oligonucleotides forming arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Polynucleotides or oligonucleotides may also be noncovalently immobilized on the substrate by hybridization to anchors, by means of magnetic beads, or in a fluid phase such as in microtiter wells or capillaries. Arrays or microarrays of polynucleotides are generally nucleic acids such as DNA, RNA, PNA, and cDNA but may also include proteins, polypeptides, oligosaccharides, cells, tissues and any permutations thereof which can specifically bind the amplification products.

Methods of detecting detectable signals are known in the art. Signal detection may be visual or utilize a suitable instrument appropriate to the particular label used, such as a spectrometer, fluorimeter, or microscope. For example, where the label is a radioisotope, detection can be achieved using, for example, a scintillation counter, or photographic film as in autoradiography. Where a fluorescent label is used, detection may be by exciting the fluorochrome with the appropriate wavelength of light and detecting the resulting fluorescence, such as by microscopy, visual inspection or photographic film. Where enzymatic labels are used, detection may be by providing appropriate substrates for the enzyme and detecting the resulting reaction product. Simple colorimetric labels can usually be detected by visual observation of the color associated with the label; for example, conjugated colloidal gold is often pink to reddish, and beads appear the color of the bead.

In one embodiment, the amplification products in a reaction mixture are analyzed on a suitable matrix. In some embodiments, separation of the amplification products from the reaction mixture is performed before they are contacted with the array. Any of a number of methods can be used to effect the separation, as described in, for example, McIntosh et al. (PCT Pub. No. WO98/59066). Such methods include, but are not limited to, mass spectrometry, flow cytometry, HPLC, FPLC, size exclusion chromatography, affinity chromatography, and gel electrophoresis.

Depending on the sensitivity of the detection method, the limits of detection of the methods of the invention are a minimum of 1 analyte moiety (when combined with highly sensitive detection methods, e.g., luminescent oxygen channeling and some array-based methods), or, with less sensitive detection methods, a minimum of 10, 100, 10³, 10⁴, 10⁵, 10⁶, 10⁷, 10⁸, 10⁹, or 10¹⁰ analyte moieties.

B. Quantification

It is apparent that the primer extension- and transcription-based methods described herein may also be used to quantify an analyte in a sample. The amount of amplification product produced is linearly related to the amount of analyte in the sample. Thus, in some embodiments, comparison of amount of amplification product obtained in a test sample with the amount of amplification product obtained in a reference sample comprising a known amount of an analyte provides quantification of the analyte in the test sample. Methods of making such comparisons are known in the art. One of skill in the art will appreciate that it is also possible to quantify an analyte by using a reference comprising a known amount of binding partner, without analyte. As is evident, as used herein, “quantification” refers to the determination of an absolute level of an analyte (for example, amount of an analyte and/or binding partner in a sample as measured by number of copies or weight), as well as a relative levels of an analyte in a sample. In one embodiment, amount of an analyte is compared to amount of another analyte. Thus, quantification of an analyte also includes the determination of the relative level of two or more analytes, for example, of bound and unbound ligand. Comparison of the amount of amplification product containing a first detectable identifying characteristic obtained in a test sample and the amount of amplification product containing a second detectable identifying characteristic obtained in the same test sample permits quantification of the relative amounts of each analyte. It is further appreciated that a reference label is desirably used, for example, to normalize signal intensity for incorporated labeled dNTPs (or ddNTPs) and to control for variation in experimental and/or detection conditions. A non-limiting example of a reference dye includes LIZ (ABI).

Reaction conditions, components, and other experimental parameters as well as illustrative embodiments in this section are generally as described herein.

VII. Timepoints and Complexes

In one embodiment, the foregoing components are added simultaneously at the initiation of analyte-binding partner binding. In other embodiments, components are added in any order prior to or after appropriate timepoints during the binding, amplification, or detection process, as required and/or permitted by the respective reactions. Such timepoints, some of which are noted below, can be readily identified by a person of skill in the art. The present invention encompasses complex(es) exemplified by the complexes that are present at each such timepoint.

For example, the process may be paused or halted at least at the following time points: after contacting binding partner and analyte (if present) (forming an analyte-binding partner complex and, generally, free binding partner); after separating unbound (free) binding partner from analyte-binding partner complex, if present (leaving the analyte-binding partner complex); after binding of composite primer to oligonucleotide template of the analyte-binding partner complex (forming analyte-binding partner-composite primer complex); at various timepoints in the amplification process (described in more detail below); after amplification of a portion of the oligonucleotide template (giving a mixture of analyte-binding partner-composite primer complex and amplification product); after separation of amplification product (if performed) (leaving separated amplification product suitable for detection).

Methods for stopping the reactions involved in the various steps of the methods of the invention are known in the art, including, for example, cooling the reaction mixture to a temperature that inhibits enzyme activity or heating the reaction mixture to a temperature that destroys an enzyme. Methods for resuming the reactions are also known in the art, including, for example, raising the temperature of the reaction mixture to a temperature that permits enzyme activity or replenishing a destroyed (depleted) enzyme. In some embodiments, one or more of the components of the reactions is replenished prior to, at, or following the resumption of the reactions. Alternatively, the reaction can be allowed to proceed (i.e., from start to finish) without interruption.

The reaction can be allowed to proceed without purification of intermediate complexes, for example, to remove unbound binding partner, or to remove primer in the amplification steps. Products can be purified at various timepoints, which can be readily identified by a person of skill in the art.

Accordingly, as would be evident to one skilled in the art, the invention includes various aspects in which a complex formed in the detection and/or quantification methods of the invention serves as a starting material for detection and/or quantification according to the invention. Complexes included within the present invention include analyte-binding partner complex, wherein at least one binding partner of the complex is attached to an oligonucleotide template. In some embodiments, the complex further comprises composite primer(s) bound to the oligonucleotide template(s) of the analyte-binding partner complex. In other embodiments, the complex further comprises primer extension product(s) bound to the oligonucleotide template(s) of the analyte-binding partner complex. In other embodiments, the complex further comprises composite primer(s) and primer extension product(s) bound to the oligonucleotide template(s) of the analyte-binding partner complex.

The invention also provides methods for detecting and/or quantifying an analyte in a sample by amplifying a polynucleotide sequence complementary to an oligonucleotide template comprising: (a) hybridizing a polynucleotide comprising a termination polynucleotide sequence to an oligonucleotide template-composite primer complex, wherein said oligonucleotide template is bound to a binding partner (in some embodiments, in an analyte-binding partner complex), and wherein said complex comprises a composite primer hybridized to a single stranded oligonucleotide template comprising the primer extension region, said composite primer comprising an RNA portion and a 3′ DNA portion, whereby said polynucleotide comprising a termination polynucleotide sequence is hybridized to a region of the oligonucleotide template which is 5′ with respect to hybridization of the composite primer to the oligonucleotide template; (b) extending the composite primer in the oligonucleotide template-composite primer complex of step (a) with DNA polymerase; (c) cleaving RNA of the annealed composite primer with an enzyme that cleaves RNA from an RNA/DNA hybrid such that another composite primer hybridizes to the oligonucleotide template and repeats primer extension by strand displacement, whereby multiple copies of the complementary sequence of the oligonucleotide template are produced.

In another embodiment, the invention provides methods for amplifying a polynucleotide sequence complementary to an oligonucleotide template that is bound to a binding partner (in some embodiments, in an analyte-binding partner complex) comprising: (a) extending a composite primer in a complex comprising (i) a single stranded oligonucleotide template comprising the primer extension region, wherein said oligonucleotide template is bound to a binding partner (in some embodiments, in an analyte-binding partner complex); and (ii) the composite primer, said composite primer comprising an RNA portion and a 3′ DNA portion, wherein the composite primer is hybridized to the oligonucleotide template; (b) cleaving RNA of the annealed composite primer with an enzyme that cleaves RNA from an RNA/DNA hybrid such that another composite primer hybridizes to the oligonucleotide template and repeats primer extension by strand displacement, whereby multiple copies of the complementary sequence of the oligonucleotide template are produced.

In still another embodiment, the invention provides methods for amplifying a polynucleotide sequence complementary to an oligonucleotide template that is attached to a binding partner (in some embodiments, in an analyte-binding partner complex) comprising: (a) extending a composite primer in a complex comprising (i) a single stranded oligonucleotide template comprising the primer extension region, wherein said oligonucleotide template is bound to a binding partner (in some embodiments, in an analyte-binding partner complex); (ii) the composite primer, said composite primer comprising an RNA portion and a 3′ DNA portion, wherein the composite primer is hybridized to the oligonucleotide template; and (iii) a polynucleotide comprising a termination polynucleotide sequence, wherein the polynucleotide comprising a termination polynucleotide sequence is hybridized to a region of the template which is 5′ with respect to hybridization of the composite primer to the oligonucleotide template; (b) cleaving RNA of the annealed composite primer with an enzyme that cleaves RNA from an RNA/DNA hybrid such that another composite primer hybridizes to the oligonucleotide template and repeats primer extension by strand displacement, whereby multiple copies of the complementary sequence of the oligonucleotide template are produced.

In embodiments of the invention wherein displaced primer extension product is generated, the methods can further comprise hybridizing a polynucleotide comprising a propromoter and a region which hybridizes to the displaced primer extension product under conditions which allow transcription to occur by RNA polymerase, such that RNA transcripts are produced comprising sequences complementary to the displaced primer extension products; whereby multiple copies of the oligonucleotide template are produced.

For example, in yet another embodiment, the invention provides methods for amplifying an oligonucleotide template attached to a binding partner (in some of these emboiments, in an analyte-binding partner complex) comprising: hybridizing a primer extension product with a polynucleotide comprising a propromoter and a region which hybridizes to the primer extension product under conditions which allow transcription to occur by RNA polymerase, such that RNA transcripts are produced comprising sequences complementary to the primer extension product, wherein the primer extension product is a displaced primer extension product generated by: (a) hybridizing an oligonucleotide template that is attached to a binding partner (in some embodiments, in an analyte-binding partner complex) with a composite primer, said composite primer comprising an RNA portion and a 3′ DNA portion; (b) optionally hybridizing a polynucleotide comprising a termination polynucleotide sequence to a region of the oligonucleotide template which is 5′ with respect to hybridization of the composite primer to the template; (c) extending the composite primer with DNA polymerase; (d) cleaving RNA of the annealed composite primer with an enzyme that cleaves RNA from an RNA/DNA hybrid such that another composite primer hybridizes to the oligonucleotide template and repeats primer extension by strand displacement to produce displaced primer extension product; whereby multiple copies of at least a portion of the oligonucleotide template are produced.

VIII. Compositions, Kits, and Systems of the Invention

The invention also provides compositions and kits used in the methods described herein. The compositions may be any component(s), complex, reaction mixture and/or intermediate described herein, as well as any combination thereof.

For example, the invention provides a composition comprising a complex of (a) a binding partner attached to an oligonucleotide template and (b) a composite primer, wherein the composite primer comprises an RNA portion and a 3′ DNA portion. In another example, the invention provides a composition comprising a complex of (a) a binding partner attached to an oligonucleotide template and (b) a composite primer, wherein the composite primer comprises a 5′-RNA portion and a 3′-DNA portion. In one embodiment, the RNA portion is adjacent to the DNA portion. In another example, the invention provides a composition comprising a complex of (a) a binding partner attached to an oligonucleotide template and (b) a composite primer, wherein the composite primer comprises 5′- and 3′-DNA portions with at least one intervening RNA portion. In other examples, the invention provides a composition comprising a complex of (a) a binding partner attached to an oligonucleotide template and (b) a composite primer that is further derivatized by attachment of a moiety capable of effecting attachment of a polynucleotide comprising the composite primer to a solid substrate used in preparing nucleic acid microarrays. In some embodiments, the composite primer is further derivatized by attachment of a positively charged moiety such as an amine. In other embodiments, the invention provides a composition comprising a complex of (a) a binding partner attached to an oligonucleotide template and (b) a TSO (i.e., any of the TSO embodiments described herein, including TSOs containing one or more modifications which enhance binding to template). In some embodiments, the compositions comprise a composite primer and a termination sequence. In some embodiments, the invention provides a composition comprising a complex of (a) a binding partner attached to an oligonucleotide template and (b) a polynucleotide comprising a propromoter sequence, such as a TSO or PTO (i.e., any of those embodiments described herein), and may further comprise a composite primer and/or a blocker sequence. In some embodiments, the invention provides a composition comprising a complex of (a) a binding partner attached to an oligonucleotide template and (b) a blocker sequence (i.e., any of the embodiments described herein, including blocker sequences with modifications). Any of these complexes (and any of the complexes described in this section) can further comprise analyte(s).

In some embodiments, e.g., those employing “sandwich” methods, the invention provides a composition comprising a complex of (a) an intermediate binding partner specific for an analyte; (b) a binding partner attached to an oligonucleotide template, wherein said binding partner is specific for the intermediate binding partner and (c) a composite primer, wherein the composite primer comprises an RNA portion and a 3′ DNA portion. In another example, the invention provides a composition comprising a complex of (a) an intermediate binding partner specific for an analyte; (b) a binding partner attached to an oligonucleotide template, wherein said binding partner is specific for the intermediate binding partner and (c) a composite primer, wherein the composite primer comprises a 5′-RNA portion and a 3′-DNA portion. In one embodiment, the RNA portion is adjacent to the DNA portion. In another example, the invention provides a composition comprising a complex of (a) an intermediate binding partner specific for an analyte; (b) a binding partner attached to an oligonucleotide template, wherein said binding partner is specific for the intermediate binding partner and (c) a composite primer, wherein the composite primer comprises 5′- and 3′-DNA portions with at least one intervening RNA portion. In other examples, the invention provides a composition comprising a complex of (a) an intermediate binding partner specific for an analyte; (b) a binding partner attached to an oligonucleotide template, wherein said binding partner is specific for the intermediate binding partner and (c) a composite primer that is further derivatized by attachment of a moiety capable of effecting attachment of a polynucleotide comprising the composite primer to a solid substrate used in preparing nucleic acid microarrays. In some embodiments, the composite primer is further derivatized by attachment of a positively charged moiety such as an amine. In other embodiments, the invention provides a composition comprising a complex of (a) an intermediate binding partner specific for an analyte; (b) a binding partner attached to an oligonucleotide template, wherein said binding partner is specific for the intermediate binding partner and (c) a TSO (i.e., any of the TSO embodiments described herein, including TSOs containing one or more modifications which enhance binding to template). In some embodiments, the compositions comprise a composite primer and a termination sequence. In some embodiments, the invention provides a composition comprising a complex of (a) an intermediate binding partner specific for an analyte; (b) a binding partner attached to an oligonucleotide template, wherein said binding partner is specific for the intermediate binding partner and (c) a polynucleotide comprising a propromoter sequence, such as a TSO or PTO (i.e., any of those embodiments described herein), and may further comprise a composite primer and/or a blocker sequence. In some embodiments, the invention provides a composition comprising a complex of (a) an intermediate binding partner specific for an analyte; (b) a binding partner attached to an oligonucleotide template, wherein said binding partner is specific for the intermediate binding partner and (c) a blocker sequence (i.e., any of the embodiments described herein, including blocker sequences with modifications).

In other embodiments, the invention provides compositions comprising a complex of (a) a binding partner attached to an oligonucleotide template; (b) a composite primer, wherein the composite primer comprises an RNA portion and a 3′ DNA portion (in some embodiments, the RNA portion is adjacent to the DNA portion); and (c) a termination sequence. In some embodiments, the termination sequence is a TSO. In other embodiments, the termination sequence is a blocking sequence. In some embodiments, the composite primer comprises a 5′-RNA portion and a 3′-DNA portion (in certain embodiments, the RNA portion is adjacent to the DNA portion). In other embodiments, the composite primer comprises 5′- and 3′-DNA portions with at least one intervening RNA portion. In some embodiments, the composition comprises a complex of (a) a binding partner attached to an oligonucleotide template; (b) a composite primer; (c) a polynucleotide comprising a termination sequence; (d) a polynucleotide comprising a propromoter sequence. In some embodiments, the propromoter sequence is provided by a PTO. In other embodiments, the propromoter sequence is provided by a TSO. Any of the above compositions may further comprise any of the enzymes described herein (such as DNA polymerase, RNaseH, and/or RNA polymerase). The compositions are generally in aqueous form, preferably in a suitable buffer.

The invention also provides compositions comprising the amplification products described herein. Accordingly, the invention provides a population of DNA (sense or anti-sense) or RNA (sense) molecules which are copies of an oligonucleotide template attached to a binding partner, which are produced by any of the methods described herein.

The compositions are generally in a suitable medium, although they can be in lyophilized form. Suitable media include, but are not limited to, aqueous media (such as pure water or buffers).

The invention provides kits for carrying out the methods of the invention. Accordingly, a variety of kits are provided in suitable packaging. The kits may be used for any one or more of the uses described herein, and, accordingly, may contain instructions for any one or more of the following uses: contacting analyte and binding partner under conditions suitable for formation of analyte-binding partner complex; amplifying a nucleotide sequence; detection of amplification products; and quantification of amplification products.

The kits of the invention comprise one or more containers comprising any combination of the components described herein, and the following are examples of such kits. A kit may comprise any of the binding partners and composite primers described herein. A kit may comprise a binding partner attached to an oligonucleotide template or have these components separately provided (generally with instructions as to how to effect attachment). For example, a kit may comprise an antibody specific to a member of the Botulinum toxin (BoNT) family. In some embodiments, a kit comprises two or more binding partners and/or two or more composite primers, which may or may not be separately packaged. In other embodiments, a kit comprises a binding partner, a composite primer and a termination sequence (any of those described herein). A kit may comprise a binding partner, a composite primer, a polynucleotide comprising a termination sequence, and a polynucleotide comprising a propromoter sequence (which may be a PTO or TSO). The composite primer may be labeled or unlabeled. Kits may also optionally include an intermediate binding partner specific for an analyte, and/or any of one or more of the enzymes described herein, as well as deoxynucleoside triphosphates and/or ribonucleoside triphosphates. Kits may also include one or more suitable buffers (as described herein). Kits useful for producing labeled amplification products may optionally include labeled or unlabelled nucleotides or nucleotide analogs. Kits may further provide a capture moiety and/or solid surface. One or more reagents in the kit can be provided as a dry powder, usually lyophilized, including excipients, which on dissolution will provide for a reagent solution having the appropriate concentrations for performing any of the methods described herein. Each component can be packaged in separate containers or some components can be combined in one container where cross-reactivity and shelf life permit.

The kits of the invention may optionally include a set of instructions, generally written instructions, although electronic storage media (e.g., magnetic diskette or optical disk) containing instructions are also acceptable, relating to the use of components of the methods of the present invention for the intended analyte detection and/or quantification, nucleic acid amplification, and/or, as appropriate, for using the amplification products for purposes such as detection and quantification. The instructions included with the kit generally include information as to reagents (whether included or not in the kit) necessary for practicing the methods of the presentation invention, instructions on how to use the kit, and/or appropriate reaction conditions.

The component(s) of the kit may be packaged in any convenient, appropriate packaging. The components may be packaged separately, or in one or multiple combinations. Where kits are provided for practicing analyte detection using the transcription-based enhanced linear amplifications methods of the present invention, the RNA polymerase (if included) is preferably provided separately from the components used in the steps prior to the transcription steps.

The relative amounts of the various components in the kits can be varied widely to provide for concentrations of the reagents that substantially optimize the reactions that need to occur to practice the methods disclosed herein and/or to further optimize the sensitivity of any assay.

The invention also provides systems for effecting the methods described herein. These systems comprise various combinations of the components discussed above. For example, in some embodiments, the invention provides a system suitable for detecting and/or quantifying an analyte comprising (a) a binding partner specific for the analyte, attached to an oligonucleotide template, (b) a composite primer (any of those described herein), (c) DNA polymerase; and (d) ribonuclease. In some embodiments, the system further comprises a polynucleotide comprising a termination sequence (any of those described herein). In some embodiments, the system further comprises a polynucleotide comprising a propromoter sequence (which may be a PTO or TSO) and a DNA-dependent RNA polymerase. Any of the systems embodiments may also comprise one or more intermediate binding partners, as described herein.

The invention also provides reaction mixtures (or compositions comprising reaction mixtures) which contain various combinations of components described herein. In some embodiments, the invention provides reaction mixtures comprising (a) a binding partner, such as an antibody or antibody derviative, attached to an oligonucleotide template; (b) a composite primer comprising a 3′ DNA portion and an RNA portion; and (c) DNA polymerase. As described herein, any of the composite primers may be in the reaction mixture (or a plurality of composite primers), including a composite primer comprises a 5′ RNA portion which is adjacent to the 3′ DNA portion. The reaction mixture could also further comprise an enzyme which cleaves RNA from an RNA/DNA hybrid, such as RNase H. A reaction mixture of the invention can also comprise any of the polynucleotides comprising termination sequences described herein. Another example of a reaction mixture is (a) a displaced primer extension product (which, as such, contains at its 5′ end sequence complementary to the 3′ DNA portion of the composite primer, but not sequences complementary to the RNA portion of the composite primer); (b) a polynucleotide comprising a propromoter sequence (for example, a PTO); and (c) RNA polymerase. Other reaction mixtures are described herein and are encompassed by the invention. For example, any reaction mixture may further comprise one or more analytes. A reaction mixture may also comprise one or more intermediate binding partners.

IX. Methods Using the Detection and Quantification Methods of the Invention

The methods and compositions of the invention can be used for a variety of purposes. Methods of determining the presence or absence of analyte(s) and analysis and/or comparison of multiple analytes through hybridization of amplification product to microarrays have been described above. Methods of diagnosis of infectious diseases, genetic defects and cancer, as well as other types of diagnosis (e.g., ultrasensitive detection of antibodies or antigens in plasma, blood, urine, or other samples); forensics; drug testing; detection of biowarfare agents; detection of metabolic or pathological state of a cell, cells, tissue, organ, or organism by detection of presence or absence of analyte(s) corresponding to such a state, and the like, are also possible. As will be apparent from the foregoing, the “sample” in which the presence, absence, and/or quantity of analyte is detected depends on the application for which the methods of the invention are used. For example, samples for diagnosis or for forensics include, but are not limited to, blood, plasma, serum, skin, muscle, or other tissue or organ samples, saliva, urine, feces, semen, vaginal secretions, and any other sample useful for diagnosis or forensic analysis, as will be readily apparent to those of skill in the art . Samples for biowarfare agent detection include, but are not limited to, air, soil, clothing, building materials, transportation materials, missile components, and the like. Samples may be prepared for analysis using methods well known in the art. In some embodiments one or more isolation steps are performed to enrich concentration of analyte and/or reduce contaminants or other components which may interfere with the assay. Analyte may be modified by chemical or other means to enhance binding or other characteristics advantageous in the methods of the invention. Other applications will be readily apparent to one of skill in the art.

The methods of the invention are specially suited for integration with a miniaturized microfluidics device for the detection and identification of bio-warfare agents, such as anthrax or botulinum. Current DNA-based assays and devices require the incorporation of a miniaturized temperature cycling component for performance of PCR based sequence amplification. The ability to integrate a highly efficient isothermal amplification in these devices will greatly reduce device complexity. Moreover, the single stranded amplification product is suitable for both homogeneous and heterogeneous detection. Various antibodies employed in the detection of multiple antigens may be conjugated with defined oligonucleotides, each comprising a specific nucleotide sequence and a common sequence complementary to a common SPIA™ primer. The detection of specific threat agents, such as the BoNT, may be carried out using specific antibody pairs for capture and detection. The detection of captured antigen (toxin) is carried out by SPIA™ amplification. A common SPIA™ primer may be employed for amplification of a single or a plurality of oligonucleotide templates, which may be detected by various means. When multiple antigens are detected simultaneously, the various amplification products may be detected using an array of immobilized oligonucleotides each corresponding (e.g., complementary) to a specific primer extension portion of an oligonucleotide sequence. Various array compositions have been described in recent years, which may be integrated with the rapid detection system. The hand-held micro-device is suitable for use in field-testing by emergency personnel as well as in urgent care facilities.

All references cited herein, including patent applications and publications, are incorporated by reference in their entirety.

The following Examples are provided to illustrate, but not limit, the invention.

### EXAMPLES

### Example 1

Single Primer Isothermal Amplification

This example illustrates the accuracy and amplification power of single primer isothermal amplification.

Various target human and bacterial (E. coli, M13) genomic DNA sequences as well as synthetic single-stranded control sequences were amplified using the single primer isothermal amplification (SPIA™) method. See U.S. Pat. No. 6,251,639. SPIA™ was carried out as follows: A single composite primer, comprising a 3′ DNA portion and a 5′ RNA portion, was used for amplification of a defined sequence. Specific composite primers for the control and test nucleic acid sequences were employed. The design of the sequence specific composite primers was carried out using commercially available software programs, as used for PCR primers, and the primers were prepared by Dharmacon. The reaction was carried out in Tris buffer at pH 8.5, 0 to 50 mM KCl, 2 to 5 mM MgCl₂, 0.25 to 0.5 mM dNTPs, a DNA polymerase with strong strand displacement activity, such as Bca or Bst DNA polymerases, RNase H, and 1 to 5 mM DTT, 3 ug T4gp32 (USB), BSA, and Rnasine. The reaction mixtures comprising the primers, samples and/or controls, were first denatured by incubation at 95° C. for 2 to 5 min., and the primer(s) were allowed to anneal to the respective target by incubation at 55° C. for 5 min. The enzyme mixture was than added to the reaction tubes and the amplification and signal generation and detection was carried out by further incubation at this temperature for 30 min.

The amplification efficiency of various specific genomic and synthetic targets was determined by quantification of the amplification products using the Quantitative Real Time PCR method. The quantification reactions were carried out using primer pairs designed to amplify the specific products, commercially available kits for quantitative Real Time PCR and BioRad iCycler equipped with fluorescent detectors. Further characterization of SPIA™ amplification products of specific target sequences was carried out by sequence determination, using cycle sequencing and determination of the product sequence using capillary electrophoresis (ABI Prism® 370 Genetic Analyzer). An example of the alignment of the detected sequence of a SPIA™ amplification product of a control synthetic target (sequence 221) with the expected sequence is shown below. The amplification product used in the cycle sequencing reaction was generated by SPIA™ amplification of 103 molecules of the target sequence. Dotted line denotes the sites of primer binding. Perfect sequencing results were obtained for the amplification product, indicating the high fidelity and efficiency of SPIA™ sequence amplification reaction.

Syn221 TCTCAGGTTT CAGGGATTAG GGAGATATTA TTTGGCCAAA CACACAAACGSPIA ™ prod.
.................. GGAGATATTA TTTGGCCAAA CACACAAACG
Syn221 GAGATGAAAA GGGAAAGATG TGCCAGATAC TGGGGAGCCT TGGAGGGTTGSPIA ™ prod
GAGATGAAAA GGGAAAGATG TGCCAGATAC TGGGGAG..............

Assessment of the amplification efficiency was generally carried out by quantification of the specific product using a commercially validated method, such as Real Time PCR, using primer-pairs designed to anneal to the predicted SPIA™ product sequence. In addition, the sequences of the various specific products were also determined using cycle sequencing (ABI kits and instrumentation). Amplification efficiency for a synthetic DNA control target is shown in Table 1. Similar SPIA™ amplification efficiencies were obtained for amplification of genomic sequences.

TABLE 1
SPIA ™ efficiency of amplification of a synthetic control sequence
221, determined by Real Time PCR for quantification of SPIA ™
amplification products.

| Input Copy Numbers | Amplification Efficiency |
|---|---|
| 2 × 10⁸ | 2 × 10⁵ |
| 2 × 10⁷ | ^{>}2 × 10⁵ |
| 2 × 10⁶ | ^{>}2 × 10⁶ |
| 2 × 10⁵ | ^{>}2 × 10⁶ |
| 2 × 10⁴ | ^{>}2 × 10⁷ |
| 2 × 10³ | ^{>}2 × 10⁷ |

This example illustrates the fidelity and amplification power of SPIA™ for rapid amplification and detection of specific nucleic acid sequence tags.

### Example 2

SPIA™-Enhanced Immunoassay of TNFα

This example illustrates the detection and quantification of human TNFα, using commercially available monoclonal antibodies and a synthetic DNA template as a tag.

Oligonucleotide templates are synthesized and conjugated to specific antibodies using thiol chemistry as previously described (Wiltshire et al, 2000, Detection of Multiple Allergen-specific IgEs on Microarrays by Immunoassay with Rolling Circle Amplification, Clin Chem 46:1990-1993). Anti-human TNFα monoclonal antibodies and antigen are obtained from BD Biosciences (Pharmingen, http://www.bdbiosciences.com/ptProductList.jsp?page=14). Thiol modified synthetic target oligodeoxynucleotides is obtained from Operon, and is conjugated to the specific antibody using published procedures (Operon). The synthetic target DNA is designed with a 3′ sequence corresponding to a well-characterized SPIA™ primer known to yield highly efficient amplification. The immune complex is formed in the reaction mixture and is captured on solid support by contacting the solution with solid surface comprising an immobilized second antibody, i.e. anti TNF monoclonal antibody, to form a tri molecular complex of the analyte bound to the two antibodies, one of which is labeled with the target nucleic acid. The free labeled complex is washed away and the solid surface is combined with the amplification reaction mixture. The amplification reaction mixture comprises a composite primer, polymerase, RNase H, dNTPs and buffer components, as described above.

Various methods for the detection and quantification of the amplification products are used. For example, detection of SPIA™ amplification products using fluorescent probe hybridization methods, such as Molecular Beacons, is used. The use of sequence specific Molecular Beacon probes for real time detection of amplification products affords sensitive detection of specific amplification products and has been described (Tyagi S and Kramer F, 1996, Molecular beacons: probes that fluoresce upon hybridization. Nat. Biotechnol. 14:303-308). These methods are used for detection of the products of the SPIA™-enhanced immunoassay.

### Example 3

SPIA™-Enhanced Immunoassay for BoNT.

This example demonstrates an assay system directed at the detection of multiple analytes that allows the detection of any of a group of specific toxins.

BoNT are among the most potent toxins known, with an LD₅₀ in the range of 1 to 5 ng kg⁻¹. Pairs of antibodies specific for specific BoNT are obtained and tested for specificity and sensitivity of a non-enhanced ELISA assay, to select for suitable capture and detector antibody pairs. The capture antibody is the antibody immobilized on a solid surface, such as a well of a microtiter plate, or a bead. The detector antibody is the antibody conjugated to the target nucleic acid reporter. Both antibodies recognize the analyte but are not competing for the same recognition site. Affinity purified antibodies for specific toxins and the corresponding toxins are commercially available from MetaBiologics Inc. (http)://www.metabiologics.com/products.htm). BoNT-specific monoclonal antibodies suitable for detection of the specific botulinum toxins are also used. These are provided by Dr. James Marks at the University of California, San Francisco. In addition, combinations of affinity purified polyclonal antibodies and monoclonal antibodies are assessed for maximal assay performance. The preparation of antibody-target (tag) DNA conjugates is performed as defined for the TNFα system and the performance of the SPIA™-enhanced immunoassay is performed using purified antigen. Then the method is assessed for the detection and quantification of specific BoNT in samples (e.g. soil, water, biofluids, etc.) that mimic field conditions.

Although the foregoing invention has been described in some detail by way of illustration and example for purposes of clarity and understanding, it will be apparent to those skilled in the art that certain changes and modifications may be practiced.

Therefore, descriptions and examples should not be construed as limiting the scope of the invention, which is delineated by the appended claims.

What is claimed is:

1. A method for determining presence or absence of an analyte in a sample comprising:
a) contacting the sample with a binding partner that is attached to an oligonucleotide template and that is capable of binding, directly or indirectly, to the analyte, if present, under conditions that permit binding, whereby an analyte-binding partner complex is formed if analyte is present;
b) separating analyte-binding partner complex from unbound binding partner;
c) amplifying a polynucleotide sequence complementary to at least a portion of the oligonucleotide template according to a method comprising:
(i) hybridizing a composite primer to the oligonucleotide template, said composite primer comprising an RNA portion and a 3′ DNA portion;
(ii) extending the composite primer with DNA polymerase, whereby a primer extension product comprising a detectable identifying characteristic is produced;
(iii) cleaving RNA of the hybridized extended composite primer with an enzyme that cleaves RNA from an RNA/DNA hybrid such that another composite primer hybridizes to the oligonucleotide template and repeats primer extension by strand displacement to produce cleaved primer extension product that comprises a detectable identifying characteristic;
whereby multiple copies of the polynucleotide sequence complementary to at least a portion of the oligonucleotide template are produced; and
whereby detection of the cleaved primer extension product comprising the detectable identifying characteristic indicates presence of the analyte in the sample.

2. The method of claim 1, wherein said detectable identifying characteristic is selected from the group consisting of size of the cleaved primer extension product, sequence of the cleaved primer extension product, and detectable signal associated with the cleaved primer extension product.

3. The method of claim 2, wherein the detectable identifying characteristic comprises the sequence of the cleaved primer extension product, wherein the sequence is detected by hybridizing the cleaved primer extension product with a nucleic acid probe that is hybridizable to the cleaved primer extension product.

4. The method of claim 3, wherein the nucleic acid probe comprises DNA.

5. The method of claim 3, wherein the nucleic acid probe is provided as an array.

6. The method of claim 5, wherein the array comprises the probe immobilized on a substrate fabricated from a material selected from the group consisting of paper, glass, plastic, polypropylene, nylon, polyacrylamide, nitrocellulose, silicon, metal, polystyrene, and optical fiber.

7. The method of claim 2, wherein said detectable signal is associated with a label on a deoxyribonucleoside triphosphate or analog thereof that is incorporated during primer extension.

8. The method of claim 2, wherein said detectable signal is associated with interaction of two labels, wherein the labels are on deoxynucleoside triphosphates or analogs thereof, and wherein one or both of the labels is incorporated during primer extension.

9. The method of claim 8, wherein one label is on a deoxyribonucleoside triphosphate or analog thereof that is incorporated during primer extension and another label is on a deoxyribonucleoside triphosphate or analog thereof located in the primer portion of the primer extension product.

10. The method of claim 1 further comprising (d) quantifying the analyte in the sample by comparing amount of copies of the polynucleotide sequence complementary to at least a portion of the oligonucleotide template obtained in the sample, if any, to the amount of copies of the polynucleotide sequence complementary to at least a portion of the oligonucleotide template obtained in a reference comprising a known amount of the analyte obtained in a reference comprising a known amount of the analyte that is subjected to steps (a) to (c); whereby the comparison provides quantification of amount of analyte in the sample.

11. The method of claim 1 further comprising binding of the analyte to an intermediate binding partner, wherein the intermediate binding partner binds to the binding partner that is attached to the oligonucleotide template, whereby the binding partner that is attached to an oligonucleotide template indirectly binds to analyte via the intermediate binding partner instead of by binding directly to the analyte, and whereby an analyte-binding partner complex is formed.

12. The method of claim 11 wherein the intermediate binding partner comprises an antibody specific to the analyte.

13. The method of claim 12 wherein the binding partner that is attached to an oligonucleotide template comprises a second antibody specific to the intermediate binding partner antibody.

14. The method of claim 1, wherein the RNA portion of the composite primer that hybridizes to the oligonucleotide template is 5′ with respect to the 3′ DNA portion.

15. The method of claim 14, wherein the 5′ RNA portion is adjacent to the 3′ DNA portion.

16. The method of claim 1 wherein the oligonucleotide template comprises a ssDNA portion, and wherein the ssDNA portion has a length of about 25 to about 100 nucleotides.

17. The method of claim 1 wherein the oligonucleotide template comprises a ssDNA portion, and wherein the ssDNA portion has a length of about 25 to about 200 nucleotides.

18. The method of claim 1, wherein the enzyme that cleaves RNA from an RNA/DNA hybrid is RNase H.

19. The method of claim 13 wherein the analyte comprises a member of the Botulinum toxin (BoNT) family.

20. The method of claim 1 wherein the analyte is selected from the group consisting of proteins, polypeptides, peptides, nucleic acid segments, carbohydrates, cells, microorganisms and fragments and products thereof, an organic molecule, and an inorganic molecule.

21. The method of claim 20 wherein the analyte comprises a peptide.

22. The method of claim 21 wherein the analyte comprises a member of the Botulinum toxin (BoNT) family.

23. The method of claim 1, wherein the oligonucleotide template is covalently attached to the binding partner.

24. The method of claim 1, wherein the oligonucleotide template is non-covalently attached to the binding partner.

25. The method of claim 1 wherein the analyte-binding partner complex is immobilized on a solid surface.

26. A method for detecting the presence or absence of an analyte in a sample comprising incubating a reaction mixture, said reaction mixture comprising:
(a) a sample suspected of containing a complex of the analyte and a binding partner, wherein the binding partner is attached to an oligonucleotide template;
(b) a composite primer to the oligonucleotide template, said composite primer comprising an RNA portion and a 3′ DNA portion;
(c) a DNA polymerase, dNTPs, and an enzyme that cleaves RNA from an RNA/DNA hybrid;
wherein the incubation is under conditions that permit hybridization of the composite primer and the oligonucleotide template, oligonucleotide polymerization, and RNA cleavage, such that multiple copies of the polynucleotide sequence complementary to at least a portion of the oligonucleotide template are produced, and wherein detection of a detectable identifying characteristic of the copies of the polynucleotide sequence complementary to at least a portion of the oligonucleotide template indicates the presence of the analyte.

27. A method for generating multiple copies of a polynucleotide sequence complementary to at least a portion of an oligonucleotide template attached to a binding partner comprising:
(a) hybridizing a composite primer to the oligonucleotide template attached to the binding partner, said composite primer comprising an RNA portion and a 3′ DNA portion;
(b) extending the composite primer with DNA polymerase;
(c) cleaving RNA of the hybridized composite primer with an enzyme that cleaves RNA from an RNA/DNA hybrid such that another composite primer hybridizes to the oligonucleotide template and repeats primer extension by strand displacement,
whereby multiple copies of the polynucleotide sequence complementary to at least a portion of an oligonucleotide template attached to a binding partner are produced.

28. A method for determining presence or absence of each of a plurality of different analytes in a sample comprising:
a) contacting the sample with a plurality of different binding partners, each of which is attached to an oligonucleotide template and each of which is capable of binding one of the plurality of different analytes under conditions that permit binding, whereby an analyte-binding partner complex is formed for a particular pair of analyte and binding partner if the analyte is present, wherein the oligonucleotide template for each different binding partner comprises a primer-binding region that is common to all of the binding partners and a primer-extension region that is unique for each binding partner;
b) separating analyte-binding partner complexes from unbound binding partners;
c) amplifying a polynucleotide sequence complementary to at least a portion of each oligonucleotide template present after step b) according to a method comprising:
(i) hybridizing a composite primer to the oligonucleotide template, said composite primer comprising an RNA portion and a 3′ DNA portion;
(ii) extending the composite primer with DNA polymerase, whereby a unique primer extension product comprising a unique detectable identifying characteristic is produced for each analyte-binding partner complex;
(iii) cleaving RNA of the hybridized extended composite primer with an enzyme that cleaves RNA from an RNA/DNA hybrid such that another composite primer hybridizes to the oligonucleotide template and repeats primer extension by strand displacement to produce unique cleaved primer extension product for each different oligonucleotide template that comprises a unique detectable identifying characteristic;
whereby multiple copies of the polynucleotide sequence complementary to at least a portion of each oligonucleotide template present after step b) are produced; and
whereby detection of the unique detectable identifying characteristic of the polynucleotide sequence complementary to at least a portion of the oligonucleotide template attached to the binding partner for an analyte indicates the presence of the analyte in the sample.

29. The method of claim 28 further comprising quantifying the relative amounts of each analyte in the sample by comparing the relative amounts of polynucleotide sequence complementary to at least a portion of the oligonucleotide template attached to the binding partner in each analyte-binding partner complex.
